(19) United States
(12) Patent Application Publication
(10) Pub. No.: US 2022/0183562 A1
(43) Pub. Date: Jun. 16, 2022

(54) Systems and Methods using Nucleic Acid Photoacoustic Nanosensors for Cytokine Detection

(71) Applicant: Northeastern University, Boston, MA (US)

(72) Inventors: Heather A. Clark, Lexington, MA (US); Kevin Bardon, Holbrook, MA (US); Nicole Langlois, Millbury, MA (US)

(21) Appl. No.: 17/549,345

(22) Filed: Dec. 13, 2021

Related U.S. Application Data
(60) Provisional application No. 63/124,707, filed on Dec. 11, 2020.

(51) Int. Cl. A61B 5/00

(52) U.S. Cl. CPC A61B 5/0095

(57) ABSTRACT

A DNA-based nanosensor system performs photoacoustic detection of cytokines. Cytokine receptors and a photoacoustic reporter dye are used in the nanosensor for cytokine detection, which can be reversible, and can be performed continuously, in vivo, in real time. Upon receptor binding to the cytokine, the DNA structure of the nanosensor closes to induce controlled dye stacking for photoacoustic application/interrogation, resulting in an increase in photoacoustic signal in the presence of the detected cytokine. The open or closed state of the sensor can be determined through differential signals as detected with photoacoustic imaging. A system using the nanosensor performs simultaneous multiplexed cytokine imaging, in the form of an inline and removable system paired with a clinical photoacoustic instrument. Cytokines can be detected in animal research use and in human point-of-care use.

### RELATED APPLICATION

This application claims the benefit of U.S. Provisional Application No. 63/124,707, filed on Dec. 11, 2020. The entire teachings of the above application are incorporated herein by reference.

### GOVERNMENT SUPPORT

This invention was made with government support under Grant No. EB024186 awarded by the NIH National Institutes of Health. The government has certain rights in the invention.

### BACKGROUND

The global COVID-19 pandemic caused by infection with the novel coronavirus SARS-CoV-2 has had unprecedented and pervasive public health and economic impacts. The research community has moved at an unprecedented pace to understand the novel pathology of SARS-CoV-2 in causing COVID-19 and has established that the persistence of acute respiratory distress syndrome (ARDS) in severe COVID-19 patients is tied to a complex and heterogenous inflammatory syndrome often referred to as a “cytokine storm.” The COVID-19 cytokine storm (CS) is characterized by concomitant overproduction of proinflammatory cytokines with pronounced lymphopenia and suppression of type-I interferon responses, among other trends. Treatment with remdesivir and corticosteroids is recommended by the Infectious Diseases Society of America (IDSA) in patients with low oxygen saturation (<94%) who require mechanical ventilation or extracorporeal membrane oxygenation (ECMO) (11). Non-specific inflammatory markers like ferritin and C-reactive protein (CRP) are currently used for patient staging (i.e., systemic inflammation is present when CRP>50 mg/L and ferritin>500 ng/mL) (12). However, these markers are cannot predict the potential for CS to develop and lag behind cytokine changes by 1-2 days (6, 13). As CSs result from systemic elevations in inflammatory cytokines, serum cytokine levels would logically be an ideal biomarker for CS diagnosis and severity assessment. However, currently, there are several limitations to applying this approach in COVID-19 and other CS contexts (13).

First, cytokine assays are not widely available in the clinical setting. The enzyme-linked immunosorbent assay (ELISA) is the standard test for cytokines (14, 15). Clinics offering a cytokine test (often just for IL-6) must outsource the assay to an external facility, where delays (up to 3 days) in obtaining results render the information useless for rapidly evolving hyperinflammatory situations (12, 16). In June 2020, an FDA-EUA of the Elecsys™ IL-6 rapid assay (Roche Diagnostics, LLC) began to expand access to clinical cytokine assays, but these assays have yet to be widely adopted.

Second, it is unclear if a single time point cytokine measurement can predict CS severity. It is well-established that elevated cytokines, including IL-6, correlate with CS severity, but it is particularly challenging to interpret such measures in the absence of patients' baseline cytokine levels, which are needed to rule out other causes of inflammation. Additionally, commercial cytokine assay platforms currently lack analytical standardization and present significant single-reading variability between studies and locations (17), further complicating interpretation of a single test result. As such, monitoring cytokine trends (i.e., fold/net increase or rate of change) is likely to provide better correlation with CS severity than information from a single time point (18-20).

Third, measuring a single cytokine is of limited clinical utility. Cytokines are pleiotropic immunomodulators that act in complex networks. The level of a single cytokine cannot guide clinical decision making without interpreting that level in the context of other cytokines. Multiplexed immunoassays are likely to be the most useful for guiding patient care by stratifying patients according to pathway-specific inflammatory trends (21).

The immunopathology of COVID-19 follows a distinct set of stages linked with severity. In patients with COVID-19, responses to viral infection present in one of three phenotypes representative of the disease stage and extent: (i) “mild” in 80% of patients with asymptomatic or minor conditions that will not progress in severity; (ii) “moderate” in 15% of patients who develop pneumonia and require hospitalization with or without hypoxia and inflammation; and (iii) “severe” in 5% of patients with low oxygen saturation (<94%) who require intensive care (mechanical ventilation/ECMO) (11, 22) due to ARDS and/or systemic hyperinflammation, and who are at risk of death (23, 24). The current consensus on COVID-19 pathogenesis is summarized below:

1. SARS-CoV-2 entry is facilitated by interaction with angiotensin-converting enzyme 2 (ACE2) receptors (25) and subsequent endocytosis into the host cells (26). Viral RNA is released, then cellular machinery is exploited for replication, resulting in rapidly increasing viral load.

2. The initial immune response begins with an initial rise in levels of pro-inflammatory cytokines with resulting hypercytokinemia (1, 27-29), at levels below the threshold of a systemic CS (23). This cytokine signaling leads to elevation of acute phase reactants, e.g., CRP and ferritin, suggesting innate immune system activation. SARSCoV-2 is postulated to interact with pattern recognition receptors to activate NFκB and IRF3/7 (23, 30, 31). NFκB promotes transcription of cytokines, like IL-6, TNF-α, and IL-1β, which further trigger Th1 and Th17 inflammatory responses with secretion of IFNγ and IL-17. IRF3/7 promote a robust type-I interferon response (IFNα and IFNβ); a strong/early type-I IFN response is essential for defense against viral infection (32-34).

3. A state of immunodeficiency follows, where type-I IFNs are suppressed (via IRF3/7 downregulation), associated with an influx of neutrophils and monocytes/macrophages and lymphopenia, observed in 70-80% of patients (27, 35). CD4+ and CD8+ T cells display functional exhaustion via diminished IFNγ production; worsening lymphocyte depletion is associated with disease severity and elevated levels of IL-6, IL-10, and TNF-α (36-38).

4. Rapid clinical deterioration proceeds in severe COVID-19 at ˜7-10 days from symptom onset, manifested as dramatic symptomatic decline (ARDS, multiple organ failures) correlated with a rise in markers of acute systemic inflammation (CRP, ferritin), coagulopathy (D-dimer), and cell death (lactate dehydrogenase), among others (24, 27, 28, 35, 39). In the most severe cases, cytokines (IL-1β, IL-1Ra, IL-6, IL-10, IL-18, TNF-α, GM-CSF, etc.) are drastically elevated as a result of a CS (1, 5, 38). Direct links have been established between particular cytokine elevations (i.e., IL-6, IL-1β, TNF-α, IL-10, IL-18, etc.) and disease severity, longer hospital stays, the need for mechanical ventilation, and increased mortality risk (20, 29, 40-45).

The appropriate therapeutic intervention in COVID-19 is likely dependent on the phase of the disease. Due to the immunopathology of the SARS-CoV-2 virus, the two phases of the disease—viral replication (1st) and hyperinflammation (2nd)—require different therapeutic approaches. In the first phase where viral replication is the driving force, antiviral drugs like remdesivir have demonstrated efficacy, with the greatest benefit from earlier treatment (46). Additionally, immunostimulants in the form of IFNα/β supplementation are being evaluated (47, 48). In the second phase driven by a hyperinflammatory CS, steroids like dexamethasone and targeted anti-cytokine blockades, e.g., tocilizumab (anti-IL-6) and anakinra (anti-IL-1β), show promise (49-54). However, the timing of these therapeutic approaches is critical: administering immunosuppressants too early in the disease course may diminish the patient's innate ability to fight the infection and increase risk of bacteria or fungal secondary infections (55); stimulating the immune system or giving antivirals too late in the disease course would also not improve patient outcomes (23, 54, 56). Thus, biomarkers that enable more accurate disease staging may guide more effective treatment.

The COVID-19 CS presents a case study of how dynamic systemic cytokines can serve as biomarkers of infection, in ways that are applicable to cytokine storm syndromes beyond COVID. The term “cytokine storm” can be broadly defined as situations where the coordinated immune system becomes dysregulated, leading to the uncontrolled release of cytokines with different signatures depending on the initial trigger of the hyperinflammatory event, i.e., autoimmune, bacterial, genetic, iatrogenic, or viral (57). Two of the most common CS phenotypes are (1) “immune dysregulation” similar to cytokine release syndrome (CRS) as a toxic response to CAR T-cell immunotherapy (58-60) and (2) “macrophage activation syndrome” (MAS) associated with haemophagocytic lymphohistiocytosis (HLH) and juvenile idiopathic arthritis (JIA) among other autoinflammatory conditions (61-65). Conflicting hypotheses suggest that COVID-19 CS is driven by either: (a) immune dysregulation and over-production of IL-6, (b) an MAS-like profile driven by excess IL-1β via overactivation of NFκB66, or (c) a unique combination of both profiles via dysregulated cytokine production in the lungs with patient-to-patient variations. Additionally, IFNγ levels are elevated early and decline in later stages of severe COVID-19, with lower levels linked to a higher risk of lung fibrosis and ARDS complications (29, 38, 67). The IL-6/IFNγ ratio is proposed as a prognostic marker of severity in COVID-19 patients to guide therapeutic intervention (68).

However, clinical technology for rapid tracking of cytokines in the point-of-care setting is not yet available. There are numerous platforms for sensitive multiplexed cytokine analysis in the research setting, but currently, no platform can rapidly report cytokine levels from the patient's bedside. Current methods are based on numerous variations of an ELISA: plasma is isolated from whole blood samples and, following a series of washing and antibody incubation steps, generates a labeled antibody-antigen complex that creates a signal (optical or electrical) proportional to protein concentration. While this approach achieves high sensitivity and low detection limits (pM-fM levels), sample processing and send-out to a laboratory for analysis is required to obtain data for a single time point. Thus, existing platforms sacrifice real-time information to achieve sensitivity. To provide rapid or real time analysis (minutes-hours) of cytokine levels using existing platforms, repeated blood draws and outsourcing would be needed. This logistical obstacle is compounded by delays, of up to 3 days, in analysis at processing facilities before results are sent to the physician. Dynamic monitoring of cytokine concentrations is not practical using existing technology.

### SUMMARY

The above challenges of cytokine storms underscore the urgent need for simple point-of-care multiplexed cytokine assay tools to monitor patients over time and track specific inflammatory trends. To address this unmet need, an embodiment according to the invention leverages the power and innovation of DNA-based nanosensor technology. Specifically, an embodiment provides a photoacoustic cytokine reporter (PACyR) device based on a DNA-based photoacoustic nanosensor. The PACyR device can, for example, enable simultaneous monitoring of IL-6, IL-1, and IFNγ to detect an impending CS before it strikes and guide treatment to either (a) support the host immune system early in the disease course, or (b) suppress the CS with anti-inflammatory agents before major organ damage occurs. This can provide physicians a more comprehensive and contextual picture of the patient's inflammatory state—lowering COVID-19 mortality risk and reducing the hospital's burden by lowering patient need for mechanical ventilation or intensive care.

An embodiment according to the invention provides point-of-care monitoring of IL-6, IL-1, and IFNγ in patients with moderate-to-severe COVID-19, to enable early CS detection by tracking three of the cytokines correlated with poor disease outcomes. The knowledge gained from this triplex cytokine monitoring could guide earlier therapeutic interventions in CS, leading to improved patient survival, and be expanded to track other CS fingerprints.

Tools for rapid, multiplexed cytokine detection in accordance with embodiments of the invention can be used for patient care beyond COVID-19. The ability to stratify patients and identify immunomodulatory avenues is useful not only in COVID-19, but in other critical indications like sepsis, CAR T-cell immunotherapy, Staphylococcus aureus infections, pneumonia, HLH, and autoimmune/autoinflammatory conditions. Additionally, access to point-of-care cytokine assays can provide precision medicine by establishing patient-specific baselines, rather than using single-point measurements compared to population averages. These advancements could enable earlier evaluation of CS that could save lives by facilitating earlier treatments and more informed therapeutic monitoring.

An embodiment according to the invention provides:

a) Rapid measurement of a cytokine triad (IL-6, IL-1, and IFNγ) in whole blood samples. No wash steps or sample manipulation would be needed.

b) Photoacoustic imaging provides a detection method for point-of-care biomarker analysis. This method combines the high-contrast detail of optical imaging with the ability to image through blood/tissue and portability of ultrasound imaging, enabling analysis that is not tethered to the laboratory.

c) Inline cytokine monitoring enables continuous sampling for on-going analysis. A single device can be reused for repeated measurement of cytokines, enabling cytokine measurements to serve as another vital sign to guide patient care.

In one embodiment, a DNA-based nanosensor system performs photoacoustic detection of cytokines. Cytokine receptors and a photoacoustic reporter dye are used in the nanosensor for cytokine detection, which can be reversible, and can be performed continuously, in vivo, in real time. Upon receptor binding to the cytokine, the DNA structure of the nanosensor closes to induce controlled dye stacking for photoacoustic application/interrogation, resulting in an increase in photoacoustic signal in the presence of the detected cytokine. The open or closed state of the sensor can be determined through differential signals as detected with photoacoustic imaging. A system using the nanosensor performs simultaneous multiplexed cytokine imaging, in the form of an inline and removable system paired with a clinical photoacoustic instrument. Cytokines can be detected in animal research use and in human point-of-care use. In an embodiment according to the invention, there is provided a system for multiplexed photoacoustic detection of cytokines. The system comprises a plurality of nucleic acid-based nanosensors each comprising at least two sensor arms. Each arm of the at least two sensor arms comprises at least one photoacoustic reporter chromophore and a cytokine receptor. Each arm is configured, in the presence of a detected cytokine in a fluid, to induce stacking of the at least one photoacoustic reporter chromophore with at least one photoacoustic reporter chromophore in another one of the at least two sensor arms, thereby increasing a photoacoustic signal emitted by the nucleic acid based nanosensor, and each arm is further configured to induce unstacking of the at least one photoacoustic reporter chromophore when the cytokine is not present, thereby decreasing the photoacoustic signal emitted. The plurality of nucleic-acid based nanosensors comprise cytokine receptors for at least two different types of cytokines. A photoacoustic measurement system is configured to detect the photoacoustic signals emitted by the nucleic acid based nanosensors and to thereby permit distinguishing of a differential photoacoustic signal indicating a stacked or unstacked state of the at least one photoacoustic reporter chromophore of the nucleic acid based nanosensors, for each of the at least two different types of cytokines.

In further, related embodiments, the plurality of nucleic-acid based nanosensors may be configured to induce the stacking of the at least one photoacoustic reporter chromophore through a two-step binding process comprising a first binding of a first cytokine receptor in a first sensor arm with the detected cytokine, and a second binding of a second cytokine receptor in a second sensor arm with the complex of the first cytokine receptor and the detected cytokine. The nucleic-acid based nanosensors may be configured to detect two or more of: IFNγ, IL-6, and IL-1. The nucleic-acid based nanosensors may comprise at least two of the following pairs of cytokine receptor pairs, one cytokine receptor of each pair being conjugated on a first arm of the at least two sensor arms, and another cytokine receptor of each pair being conjugated on a second arm of the at least two sensor arms, the cytokine receptor pairs comprising: IFNγR1 and IFNγR2; IL-6R and gp130; and IL-1R1 and IL-1RAcP. The system may be configured to perform continuous real time monitoring of levels of the at least two different types of cytokines in the fluid. The nucleic-acid based nanosensors may comprise DNA origami based nanosensors; and may comprise protective shielding from biological conditions. The nucleic-acid based nanosensors may each comprise a tweezer structure comprising two arms attached at a central hinge configured to perform the inducing of the stacking in the presence of the detected cytokine, and the inducing of the unstacking when the cytokine is not present, and may comprise modification sites upon which the cytokine receptors and the at least one photoacoustic reporter chromophores are installed. The nucleic-acid based nanosensors may further comprise a single stranded DNA tether between the two arms of the tweezer structure. The at least one photoacoustic reporter chromophore may comprise at least one of an indocyanine green analog, a phthalocyanine chromophore, and a BODIPY analog. The at least one photoacoustic reporter chromophore may further comprise a molecular scaffold. The system may be further configured to provide a fluid flow connection to an intravenous device configured to inject the plurality of nucleic acid-based nanosensors into the fluid. The plurality of nucleic acid-based nanosensors may comprise at least part of an injectable agent configured to permit spatially resolved imaging of at least one cytokine in a living subject. The system may further comprise a plurality of fluid channels configured to flow the fluid comprising the plurality of cytokines, the plurality of nucleic acid-based nanosensors being inside at least one fluid channel of the plurality of fluid channels, the system being configured to permit photoacoustic measurement of the fluid in the at least one fluid channel. The plurality of nucleic acid-based nanosensors may be immobilized inside the at least one fluid channel of the plurality of fluid channels, or may be flowing in injected solution inside the at least one fluid channel of the plurality of fluid channels. The system may further comprise a cassette comprising the plurality of fluid channels and the plurality of nucleic acid-based nanosensors.

In another embodiment according to the invention, there is provided a method for multiplexed photoacoustic detection of cytokines. The method comprises flowing a fluid comprising a plurality of nucleic acid-based nanosensors each comprising at least two sensor arms, each arm of the at least two sensor arms comprising at least one photoacoustic reporter chromophore and a cytokine receptor, each arm being configured, in the presence of a detected cytokine in a fluid, to induce stacking of the at least one photoacoustic reporter chromophore with at least one photoacoustic reporter chromophore in another one of the at least two sensor arms, thereby increasing a photoacoustic signal emitted by the nucleic acid based nanosensor, and each arm being further configured to induce unstacking of the at least one photoacoustic reporter chromophore when the cytokine is not present, thereby decreasing the photoacoustic signal emitted. The plurality of nucleic-acid based nanosensors comprise cytokine receptors for at least two different types of cytokines. The method further comprises performing a photoacoustic measurement of the fluid to detect the photoacoustic signals emitted by the nucleic acid based nanosensors and to thereby distinguish a differential photoacoustic signal indicating a stacked or unstacked state of the at least one photoacoustic reporter chromophore of the nucleic acid based nanosensors, for each of the at least two different types of cytokines.

In further related method embodiments, the plurality of nucleic-acid based nanosensors may be configured to induce the stacking of the at least one photoacoustic reporter chromophore through a two-step binding process comprising a first binding of a first cytokine receptor in a first sensor arm with the detected cytokine, and a second binding of a second cytokine receptor in a second sensor arm with the complex of the first cytokine receptor and the detected cytokine. The method may comprise detecting two or more of: IFNγ, IL-6, and IL-1. The nucleic-acid based nanosensors may comprise at least two of the following pairs of cytokine receptor pairs, one cytokine receptor of each pair being conjugated on a first arm of the at least two sensor arms, and another cytokine receptor of each pair being conjugated on a second arm of the at least two sensor arms, the cytokine receptor pairs comprising: IFNγR1 and IFNγR2; IL-6R and gp130; and IL-1R1 and IL-1RAcP. The method may comprise performing continuous real time monitoring of levels of the at least two different types of cytokines in the fluid. The method may further comprise injecting the plurality of nucleic acid-based nanosensors into the fluid. The plurality of nucleic acid-based nanosensors may comprise at least part of an injectable agent, the method further comprising performing spatially resolved imaging of the at least two different types of cytokines in a living subject. The method may further comprise flowing the fluid comprising a plurality of nucleic-acid based nanosensors through at least one fluid channel; and performing a photoacoustic measurement of the fluid in the fluid channel to detect the photoacoustic signals emitted by the nucleic acid based nanosensors.

In another embodiment according to the invention, there is provided a nucleic acid-based nanosensor for photoacoustic detection of cytokines. The nanosensor comprises two DNA origami-based sensor arms, each sensor arm comprising at least one photoacoustic reporter chromophore and a cytokine receptor, each sensor arm being configured, in the presence of a detected cytokine in a fluid, to induce stacking of the at least one photoacoustic reporter chromophore with at least one photoacoustic reporter chromophore in another one of the two sensor arms, thereby increasing a photoacoustic signal emitted by the nucleic acid based nanosensor, and each sensor arm being further configured to induce unstacking of the at least one photoacoustic reporter chromophore when the cytokine is not present, thereby decreasing the photoacoustic signal emitted. The two sensor arms are attached at a central hinge configured to perform the inducing of the stacking in the presence of the detected cytokine, and the inducing of the unstacking when the cytokine is not present, and comprising modification sites upon which the cytokine receptors and the at least one photoacoustic reporter chromophores are installed.

In further, related nanosensor embodiments, the nanosensor may be configured to induce the stacking of the at least one photoacoustic reporter chromophore through a two-step binding process comprising a first binding of a first cytokine receptor in a first sensor arm with the detected cytokine, and a second binding of a second cytokine receptor in a second sensor arm with the complex of the first cytokine receptor and the detected cytokine. The nanosensor may be configured to detect one or more of: IFNγ, IL-6, and IL-1. The nanosensor may comprise at least one of the following pairs of cytokine receptor pairs, one cytokine receptor of each pair being conjugated on a first arm of the at least two sensor arms, and another cytokine receptor of each pair being conjugated on a second arm of the at least two sensor arms, the cytokine receptor pairs comprising: IFNγR1 and IFNγR2; IL-6R and gp130; and IL-1R1 and IL-1RAcP. The nanosensor may comprise protective shielding from biological conditions. The nanosensor may further comprise a single stranded DNA tether between the two arms of the tweezer structure. The at least one photoacoustic reporter chromophore may comprise at least one of an indocyanine green analog, a phthalocyanine chromophore, and a BODIPY analog. The at least one photoacoustic reporter chromophore may comprise a molecular scaffold. The nucleic acid-based nanosensor may comprise at least part of an injectable agent configured to permit spatially resolved imaging of at least one cytokine in a living subject.

In another embodiment according to the invention, there is provided a device for multiplexed photoacoustic detection of cytokines. The device comprises a plurality of nucleic acid-based nanosensors each comprising at least two sensor arms, each arm of the at least two sensor arms comprising at least one photoacoustic reporter chromophore and a cytokine receptor, each arm being configured, in the presence of a detected cytokine in a fluid, to induce stacking of the at least one photoacoustic reporter chromophore with at least one photoacoustic reporter chromophore in another one of the at least two sensor arms, thereby increasing a photoacoustic signal emitted by the nucleic acid based nanosensor, and each arm being further configured to induce unstacking of the at least one photoacoustic reporter chromophore when the cytokine is not present, thereby decreasing the photoacoustic signal emitted. The plurality of nucleic-acid based nanosensors comprise cytokine receptors for at least two different types of cytokines. The device is configured to permit detection of the photoacoustic signals emitted by the nucleic acid based nanosensors by a photoacoustic measurement system and to thereby permit distinguishing of a differential photoacoustic signal indicating a stacked or unstacked state of the at least one photoacoustic reporter chromophore of the nucleic acid based nanosensors, for each of the at least two different types of cytokines.

In further, related device embodiments, the device may further comprise a plurality of fluid channels configured to flow the fluid comprising the plurality of cytokines, the plurality of nucleic acid-based nanosensors being inside at least one fluid channel of the plurality of fluid channels; the system being configured to permit photoacoustic measurement of the fluid in the at least one fluid channel. The plurality of nucleic acid-based nanosensors may be immobilized inside the at least one fluid channel of the plurality of fluid channels. The plurality of nucleic acid-based nanosensors may flow in injected solution inside the at least one fluid channel of the plurality of fluid channels. The device may further comprise a cassette comprising the plurality of fluid channels and the plurality of nucleic acid-based nanosensors.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a schematic diagram illustrating an overview of a system for measuring cytokines through photoacoustic imaging of an inline device, in accordance with an embodiment of the invention.

FIGS. 2A-2E are diagrams illustrating an experiment conducted with a DNA nanostructure in accordance with an embodiment of the invention that features two arms attached at a central hinge with modification sites to install chromophores and cytokine receptors. FIG. 2A is a schematic diagram of a DNA origami tweezer sensor. FIG. 2B is an image showing analysis for the custom dsDNA template, template after PCR amplification, and exonuclease digested ssDNA scaffold. FIG. 2C is an image showing results for assembly of the DNA-only origami tweezers using a custom ssDNA scaffold. FIG. 2D is a graph showing the results of stability studies of the DNA origami tweezers, showing that structural integrity was maintained when stored in PBS buffer over two weeks as monitored using size exclusion chromatography. FIG. 2E is an image showing resistance of the DNA nanostructure to degradation in dilute human plasma for at least 24 hours at 37° C.

FIGS. 3A-3E are diagrams showing the results of experiments to investigate the conditions under which the DOT structures are stable, in accordance with an embodiment of the invention. FIGS. 3A-3E show a summary of SEC-HPLC DNA origami tweezer stability experiments, demonstrating retained structural integrity when stored at 4° C., in FIG. 3A, after annealing for 3 weeks and, in FIG. 3B, after purification in PBS. The experiments examined the effect of washing on retaining stability in storage and found that, as shown in FIG. 3C, DNA origami tweezers will aggregate after 3 weeks when washed with PBS, and, in FIG. 3D, that they will unfold within two days when washed with annealing buffer (AB). DNA origami tweezers remain stable when stored in 5% plasma, as shown in FIG. 3E, and can be distinguished from plasma proteins by SEC.

FIG. 4 is a schematic diagram illustrating an example of a DNA routing map of ssDNA strands to form a DNA origami tweezer nanostructure, in accordance with an embodiment of the invention.

FIGS. 5A-5F are schematic diagrams illustrating variations of hinge design for a DNA origami tweezer nanostructure, in accordance with an embodiment of the invention. FIGS. 5A and 5B are schematic diagrams of the chemical structure of the DNA strands, and FIGS. 5C-5F are schematic diagrams illustrating tuning of the spacing between dyes and receptors on the DNA origami tweezer nanostructure.

FIGS. 6A-6C are schematic diagrams illustrating an experiment in accordance with an embodiment of the invention, which investigates the position of reporters on DNA origami tweezer nanosensors for maximum signal amplification. FIG. 6A is a schematic diagram for the “off” and “on”—DNA origami tweezer sensors and expected FRET trends; FIG. 6B shows a summary of FRET analysis of magnetic bead-DNA origami tweezers with one possible fluorophore position; FIG. 6C shows an example of image data for the magnetic bead-DNA origami tweezer experiment showing three ROIs—magnetic beads with DNA origami tweezers coated on the surface.

FIGS. 7A-7C are schematic diagrams illustrating operation of a DNA tweezer nanosensor, in an embodiment in accordance with the invention. FIG. 7A shows the nanosensor mechanism, involving opening and closing of the DNA tweezer in the absence or presence of a cytokine (respectively); FIG. 7B shows incubation of the sensors in whole blood, during which a dual-wavelength photoacoustic signal produced by the sensors is monitored over time; and FIG. 7C shows the resulting complex when the cytokine protein is bound to the receptors.

FIGS. 8A-8C are schematic diagrams illustrating a photoacoustic cytokine reporter system for point-of-care immune monitoring, in accordance with an embodiment of the invention. FIG. 8A shows photoacoustic cytokine nanosensors, as taught herein, which are immobilized in fluid channels through which there flows the whole blood of a patient. FIG. 8B, illustrates bedside, in-line immune system monitoring, in which real-time cytokine levels detected in the nanosensor flow channel device are monitored. FIG. 8C shows a graphic display that illustrates a real-time analysis of patient-specific trends, which can be generated using cytokine levels measured by the nanosensor flow channel device.

FIG. 9A is a schematic diagram of a typical scheme for indocyanine green (ICG) synthesis, which can be used to form the basis of synthesis of ICG for use in a nanosensor in accordance with an embodiment of the invention. FIG. 9B is a schematic diagram showing how the structure of ICG can be functionalized with numerous groups to tune its solubility and stacking properties, for use in a nanosensor in accordance with an embodiment of the invention.

FIG. 10 shows orientations of xanthene scaffold-conjugated ICG in both “open” and “bound” probe conformations, which can be used in a DNA nanosensor in accordance with an embodiment of the invention.

FIG. 11 is a schematic diagram illustrating immobilization of DNA origami on surface of polyethylene tubing, in accordance with an embodiment of the invention.

FIG. 12 is a schematic diagram of an inline cassette device for cytokine detection, in accordance with an embodiment of the invention.

FIGS. 13A and 13B are schematic representations of a four-arm nanosensor, which is an example of another type of nanosensor that can be used as a nanosensor in accordance with an embodiment of the invention. FIG. 13A shows the nanosensor with its arms freely moving in solution when no analyte is present. FIG. 13B shows that, upon analyte binding, the arms fold in, and the dyes are held in close proximity, causing a change in photoacoustic signal.

FIGS. 14A-14C are schematic diagrams related to binding between IFNγ and its receptors, in an experiment in accordance with an embodiment of the invention. FIG. 14A is a schematic diagram of the nanosensor two-step binding mechanism between IFNγ and its receptors; FIG. 14B is a sensogram graph for IFNγ binding to the immobilized nanosensor; and FIG. 14C is a graph showing that the nanosensors are selective toward IFNγ compared to TNFα, IL-18, and PBS.

FIGS. 15A-15E are diagrams illustrating design of a Pc chromophore, in accordance with an embodiment of the invention. FIG. 15A shows a Pc chromophore; FIG. 15B shows design of Pc-1; FIG. 15C is a representation of J-aggregate stacking of a Pc-1 dye demonstrating electron sharing (dotted lines) between π-π orbitals; FIG. 15D is a graph showing Pc-1 absorbance spectra in DMF solvent (solid line) and 99% water 1% DMF (dotted line); and FIG. 15E is a graph showing absorbance and photoacoustic spectra of Pc-1-tagged DNA.

FIGS. 16A-16C are diagrams illustrating photoacoustic responses of test sequences in an experiment in accordance with an embodiment of the invention. FIG. 16A is a schematic diagram of test sequences to evaluate chromophore stacking; FIG. 16B is a set of 3D images of the test structures' ultrasound, photoacoustic response at 780 nm, and overlay; and FIG. 16C is a graph of stacked and unstacked test responses, for photoacoustic signal and absorbance.

FIGS. 17A and 17B are diagrams showing results based on a four-arm nanosensor's photoacoustic response, in experiments in accordance with an embodiment of the invention. FIG. 17A is a set of graphs showing response of the IFNγ nanosensor (780 nm) to buffer and 10 μM IFNγ (169 μg/mL) in buffer, photoacoustic signal and absorbance. FIG. 17B is a set of images of 2D scans of the nanosensor's photoacoustic response in capillary tubes.

FIG. 18 is a schematic diagram showing an overview of use of nucleic-acid based sensors for measuring cytokines through photoacoustic imaging in vivo, in accordance with an embodiment of the invention.

FIG. 19 is a schematic diagram showing a fabrication scheme for a nanosensor loaded injectable hydrogel, in accordance with an embodiment of the invention.

### DETAILED DESCRIPTION

A description of example embodiments follows.

Multiplexed Cytokine Imaging

An embodiment according to the invention provides a device for simultaneous multiplexed cytokine imaging, in the form of an inline and removable system paired with a clinical photoacoustic instrument. This DNA-based sensing platform enables triple-analyte detection by adding sensors for IL-6 and IL-1 to an IFNγ platform. Photoacoustic dyes based on ICG can be used for enhanced measurement sensitivity. An inline sensor format can be used for human point-of-care use. A set of sensors can be used to detect three cytokines that are key predictors of an oncoming cytokine storm. The result is an inline cartridge for continuous monitoring of immune response in diseases such as COVID-19 or other infections, or non-infectious inflammatory conditions. Through continuous monitoring, therapeutic interventions can be timed to more precise dosing regimens, resulting in better outcomes.

Nanosensor technology for continuous monitoring of proteins in vivo offers the potential to revolutionize biochemical research and clinical diagnostics. A key goal for nanosensors is to enable tracking of the dynamics of biomolecule expression pertinent to disease pathogenesis or therapeutic efficacy in real time. Such technology could be particularly groundbreaking in the context of cytokine storms (CSs). CSs are systemic inflammatory responses arising from immune system overstimulation that lead to extreme toxic events, e.g., multiple organ dysfunction (1). CSs are a major contributor to the morbidity associated with response to adoptive T-cell therapies as well as pathogenic infections, e.g., SARS and MERS (1-3). Increasing evidence also suggests that development of severe cases of COVID-19 correlates with the onset of a CS (1, 4, 5). Rapid blood tests for serum levels of C-reactive protein and ferritin can identify systemic inflammation, but these tests cannot accurately predict development of a severe CS or provide a readout of therapeutic efficacy (6). Serum levels of cytokines like IFNγ, IL-1, IL-6, sIL-2Rα, IL-10, and TNF-α are significantly elevated in patients with a severe CS, even before the apparent onset of severe symptoms (2-5). These cytokines could be leveraged as biomarkers to advance patient care for COVID-19 and other conditions associated with CS (6). However, achieving clinical utility necessitates urgent development of a rapid, minimally invasive continuous measurement device to track biomarkers of CS.

To promote development of robust biomarker measurement systems, an embodiment according to the invention harnesses the power of DNA nanotechnology. Herein, there is described a DNA-based sensor to detect IFNγ (7), a key cytokine regulator of CS. DNA nanotechnology enables development of functional nanostructures with site-specific modifications based on the base-pairing rules of DNA (8). The open or closed state of the sensor produces differential signals detected with photoacoustic (PA) imaging—a minimally invasive optical imaging method with high spatiotemporal resolution (9, 10). To monitor cytokine dynamics, further embodiments provide: i) simultaneous multiplexed cytokine imaging, ii) an inline and removable system, iii) an injectable system for in vivo measurements, and iv) a tool that would enable basic clinical research into COVID-19 CS pathology to inform treatment.

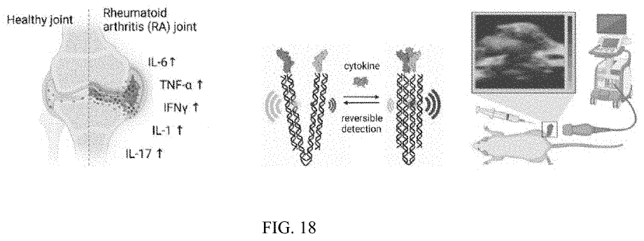

In addition, further embodiments in accordance with the invention are based on the concept that, as cytokines are pleiotropic modulators that are produced to act locally, it can be challenging to monitor the effect of systemic therapy on regulating localized cytokine activity (B5). One example where cytokine signaling microenvironments are critical to disease progression would be in the case of rheumatoid arthritis (RA). The debilitating joint pain characteristic of RA is caused by synovitis, leading to overproduction of cytokines into the synovial fluid (SF) (B6) that lead to eventual bone desorption. There are currently seven FDA-approved biologics for treatment of moderate-to-severe RA, focused on suppressing cytokines (B7). It is currently difficult to predict how a patient may respond to each strategy or to assess the progress of treatment on mitigating cytokine-specific activity in the RA-affected joints using existing technologies. Recent studies have shown that patient cytokine levels can be useful predictors of responsiveness to cytokine suppression therapies (B8, B9), with SF levels showing higher prognostic value than serum (B10). Embodiments related to the example of cytokine signaling microenvironments for rheumatoid arthritis (RA) are discussed below in connection with FIG. 18.

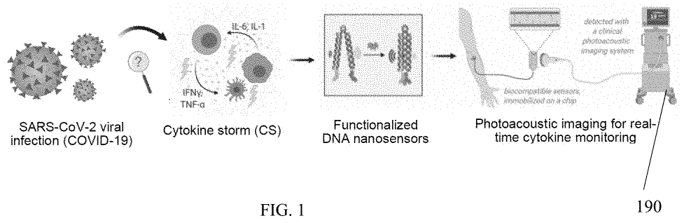

FIG. 1 is a schematic diagram illustrating an overview of a system for measuring cytokines through photoacoustic imaging of an inline device, in accordance with an embodiment of the invention. Tracking real-time dynamics of cytokines serves the purpose of prompting CS detection, early intervention, and better assessment of viral pathogenesis. With reference to the system of FIG. 1, for example, suppose that a SARS-CoV-2 viral infection (COVID-19) causes a cytokine storm (CS). This is detected by functionalized DNA nanosensors in accordance with an embodiment of the invention. The changes in the DNA nanosensors that are caused by the cytokine storm are detected by a photoacoustic imaging system 190 that images the DNA nanosensors, which are biocompatible and immobilized on a chip. The photoacoustic imaging thereby permits real-time cytokine monitoring.

Severe cases of COVID-19 exhibit elevated levels of IFNγ, IL-6, and/or IL-1, among other cytokines. As described further herein, an IFNγ sensor taught herein can be modified with existing reporters, such as Cy3 and Cy5, using the DNA origami method to improve biological stability. Sensors can be assembled with recognition units for IL-6 and IL-1, so that three sensors on a single platform can be used to perform selective detection of all three cytokines (IFNγ, IL-6, and/or IL-1), or another multiplexed group of more than one cytokine or other protein on a single platform having multiple such different DNA nanosensors.

In addition, as described further herein, modifications of indocyanine green (ICG) can be used as a photoacoustic reporter dye, in accordance with embodiments of the invention. ICG can be modified for probe attachment and enhancement of turn-on signal when a binding event occurs. A PA dye based on ICG can be used to increase optical signal when a sensor binding event “stacks” multiple dyes.

In further embodiments, nanosensors taught herein are immobilized in an inline IV device. Changes in one or more of IFNγ, IL-6, and IL-1 can be monitored using the sensors, thereby providing a system capable of inline, triple-cytokine analysis. It will be appreciated that other cytokines, and other proteins, can be monitored. The system is capable of animal research use and human point-of-care use.

In further embodiments, nanosensors taught herein are utilized as an injectable imaging agent. Changes in one or more of IFNγ, IL-6, and IL-1 can be monitored using the sensors, thereby providing a system capable of in vivo, spatially resolved cytokine analysis. It will be appreciated that other cytokines, and other proteins, can be monitored. The system is capable of animal research use and human point-of-care use.

Advantages of Photoacoustic Imaging

In accordance with an embodiment of the invention, photoacoustic imaging (PAI) enables rapid analysis in whole blood. PAI is a hybrid modality that combines the high sensitivity of optical imaging and the deep tissue penetration and resolution of ultrasonic imaging (9). This combination of high sensitivity and deep imaging depth is required by our application. In PAI, a short-nanosecond pulsed excitation beam irradiates the tissue. Absorbed energy is converted into ultrasonic waves through the thermoelastic effect. The resulting pressure rise relaxes with the propagation of a wave with energy in the ultrasound frequency range, generating pressure that may be converted to a detectable acoustic wave (73). Wideband ultrasonic transducers pick up this acoustic wave and reconstruction algorithms transform it into an image (74). The initial pressure amplitude (p₀) depends on the optical absorbance coefficient and the heat conversion efficiency (nonradiative energy decay) of the contrast agent (CA). The change in PA signal is linearly proportional to change in the optical absorption coefficient, making PAI very sensitive to the optical absorption properties of tissues or dyes such as indocyanine green (ICG) (75, 76). To maximize the signal from the proposed sensors, unwanted background signal from surrounding tissue (primarily blood) must be minimized. This is best accomplished in the NIR region, i.e., ˜700 to 1000 nm, with multi-wavelength PAI that will result in high SBR (77). Absorption of hemoglobin, light scattering from tissues, and autofluorescence from endogenous chromophores like elastin and collagen are relatively weak in the NIR (78), making it clear why this spectral range is referred to as the physiologically transparent window (79). PAI has several other advantages. PAI is modular, i.e., different sensors or molecules of interest can be PA probed by using multiple illumination wavelengths; this modularity also applies to different states, e.g., monomeric or aggregated, of the same molecules. PAI is adaptable and safe. It can be readily combined with other optical imaging modalities like optical coherence tomography, confocal microscopy, and diffuse optical tomography, as well as clinical US imaging scanners due to the same receiver electronics. Lastly, PAI does not require ionizing radiation, unlike CT or X-ray imaging. NIR wavelength maintained within safe power output ranges makes PAI a safer alternative, especially when frequent images or longer tracking times are needed (80).

DNA Origami Based Sensor

An embodiment according to the invention provides a mechanically stabilized, chemically shielded sensor. The DNA nanostructure is: (a) mechanically stabilized to limit random motion and entropically favor intra-sensor closing events, and (b) has protection strategies such as PEGylation to shield the structure in low-salt, biological conditions. The design uses developments in DNA nanotechnology, a field that involves custom nanoscale objects that undergo programmable self-assembly and are capable of performing engineered functions including drug delivery, analyte sensing, and molecular computing (83-85). The field is largely dominated by the technique of DNA origami (86), where a long single-stranded DNA (ssDNA) scaffold is precisely folded into a 2D or 3D shape via partly-complementary ssDNA staples. The advantage of using DNA origami is that the technique produces high-yielding, internally crosslinked structures of engineered form with near endless potential for site-specific chemical modifications to install function (i.e., chromophores or bioconjugation sites) (8).

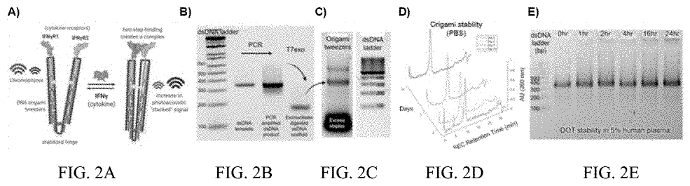

FIGS. 2A-2E are diagrams illustrating an experiment conducted with a DNA nanostructure in accordance with an embodiment of the invention that features two arms attached at a central hinge with modification sites to install chromophores and cytokine receptors, also referred to herein as a DNA origami tweezer nanosensor. FIG. 2A is a schematic diagram of a DNA origami tweezer sensor for IFNγ, using a mechanism similar to the four arm sensor taught herein. FIG. 2B is an image showing analysis using AGE (2.0%, 1× TAE) for the custom dsDNA template, template after PCR amplification, and exonuclease digested ssDNA scaffold, and, FIG. 2C is an image showing results for folding the custom ssDNA scaffold into the DNA-only origami tweezers. FIG. 2D is a graph showing the results of stability studies of the DNA origami tweezers, showing that structural integrity was maintained when stored in PBS buffer over two weeks, as evidenced by lack of peak changes on the SEC chromatogram. FIG. 2E is an image showing resistance of the DNA nanostructure to degradation in dilute human plasma for at least 24 hours, showing only minimal aggregation (<5%), highlighting the sensor stability.

The DNA construct of FIG. 2A is assembled through a custom 334-nt ssDNA scaffold that is folded into a tweezer-like shape by addition of ten partly complementary oligonucleotide staples in a 1× TE/Mg²⁺ buffer over a 3-hour temperature ramp from 94° C. to 4° C. Five, 5′-phosphate modified synthetic oligonucleotides were linked via T4 ligase to form the ssDNA scaffold, which was then amplified using PCR to produce a dsDNA template. One complementary strand in the dsDNA template was selectively digested using T7 exonuclease, as the desired strand includes a 5′-tag of five phosphorothioate-linked thymine bases (incorporated using modified PCR primers), rendering the scaffold strand resistant to exonuclease digestion (90). All strand sequences were derived from the M13mp18 phage vector genome, the traditional 7 kb ssDNA scaffold used for DNA origami (87), and modified to minimize base repeats and narrow the range of melting temperatures to impart thermal stability. This design maximizes the mechanical stability imparted by folding a uniform scaffold and minimizes the need for custom synthesis that is typically required with DNA origami involving the M13mp18 scaffold and 100-200 different synthetic staples (87). FIGS. 2B and 2C show the scaffold production workflow and origami assembly, respectively, in accordance with an embodiment of the invention. The reduced mobility of the tweezer in agarose gel electrophoresis (AGE) indicates successful assembly. The DNA origami tweezer (DOT) sensors can be purified by size exclusion chromatography (SEC) to remove excess staples, with structural integrity assessed using an analytical SEC method. Purified DOT structures exhibit structural integrity when stored in PBS buffer for over 2 weeks and demonstrate resistance to structural degradation in dilute human plasma for at least 24 hours (FIGS. 2D and 2E). This is on par with other DNA origami-based structures. The design resulted in a sensor that is stable in buffer and serum.

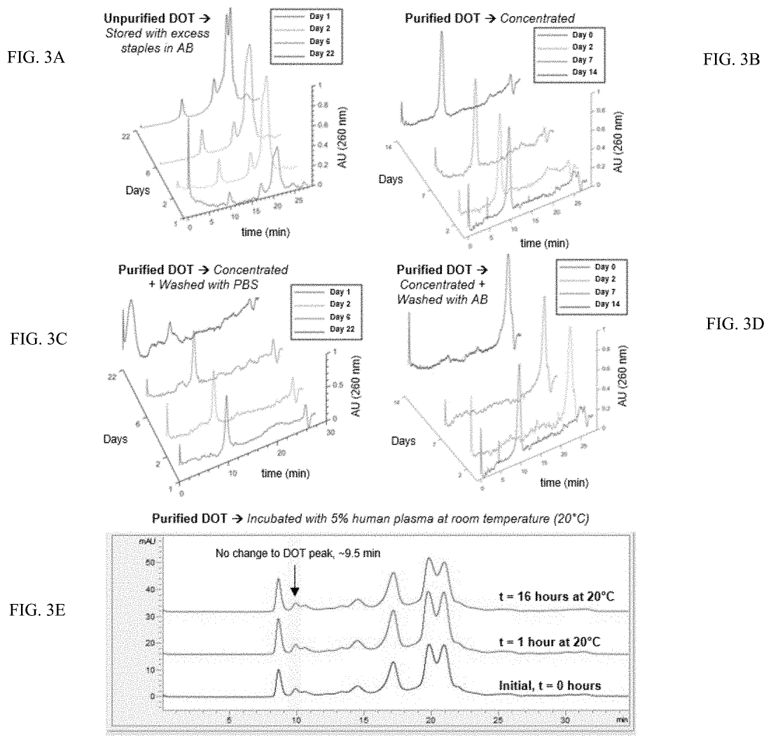

FIGS. 3A-3E are diagrams showing the results of experiments to investigate the conditions under which the DOT structures are stable, in accordance with an embodiment of the invention. FIGS. 3A-3E show a summary of SEC-HPLC DNA origami tweezer stability experiments, demonstrating retained structural integrity when stored at 4° C., in FIG. 3A, after annealing for 3 weeks and, in FIG. 3B, after purification in PBS. The experiments examined the effect of washing on retaining stability in storage and found that, as shown in FIG. 3C, DNA origami tweezers will aggregate after 3 weeks when washed with PBS, and, in FIG. 3D, that they will unfold within two days when washed with annealing buffer (AB). DNA origami tweezers remain stable when stored in 5% plasma, as shown in FIG. 3E, and can be distinguished from plasma proteins by SEC.

The results of FIGS. 3A-3E show that DOT structures can remain stable after annealing when in the presence of either excess staple strands or high-salt buffers (137 mM NaCl in 1× PBS). Washing with annealing buffer (AB, 1× TE+10 mM MgCl₂, FIG. 3D) leads to structure destabilization. This is consistent with previous studies that demonstrate that cooperative strand-displacement and salt neutralization are two strategies to maintain integrity in DNA origami. The experiments additionally show that stability of DOT in plasma can be assessed using an HPLC approach, in addition to traditional gel-based methods. The DOT remains stable in 5% human plasma for at least 16 hours as shown in FIG. 3E.

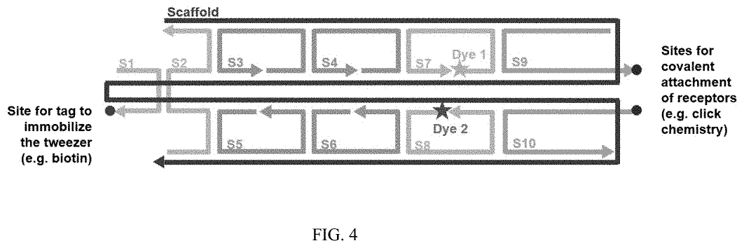

FIG. 4 is a schematic diagram illustrating an example of a DNA routing map of ssDNA strands for a DNA origami tweezer nanostructure, in accordance with an embodiment of the invention. The scaffold is a continuous single strand of DNA (in this case about 340 nt in length) that is produced through ligation of smaller strands, then enzymatic amplification. The smaller strands (staples), labeled S1 through S10, are between about 16 nt and 48 nt in length, and help to fold the longer scaffold through the DNA origami method. The routing map includes a site for a tag to immobilize the tweezer (e.g. biotin), and sites for covalent attachment of receptors, using, for example, click chemistry.

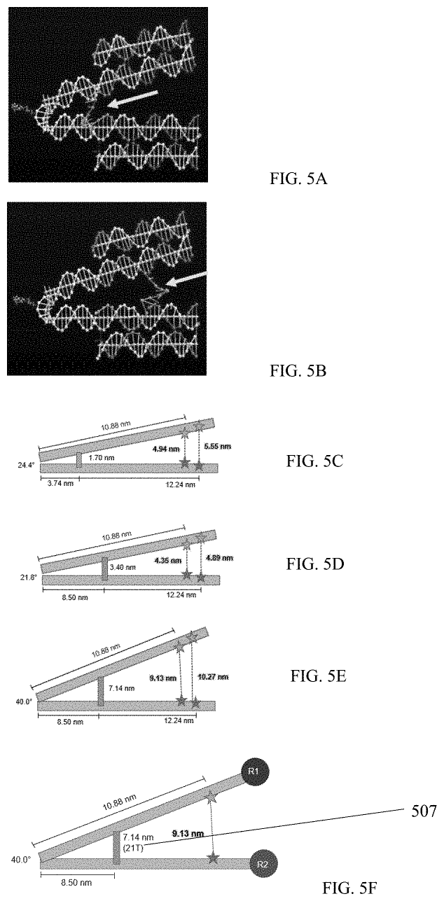

FIGS. 5A-5F are schematic diagrams illustrating possible variations of hinge design for a DNA origami tweezer nanostructure, in accordance with an embodiment of the invention. FIGS. 5A and 5B are schematic diagrams of the chemical structure of the DNA strands, and FIGS. 5C-5F are schematic diagrams illustrating tuning of the spacing between dyes and receptors on the DNA origami tweezer nanostructure. Using different lengths and routing of the DNA strands, the distance between the dyes, and thus the receptors, can be tuned, with the aid of ssDNA tethers as spacers (arrows in FIGS. 5A and 5B). For example, FIG. 5F shows one iteration, in which there is a ˜7 nm tether 507, with receptor proteins attached at the ends of the tweezer. Other dimensions are shown in FIGS. 5C-5E, and it will be appreciated that other potential dimensions can be used.

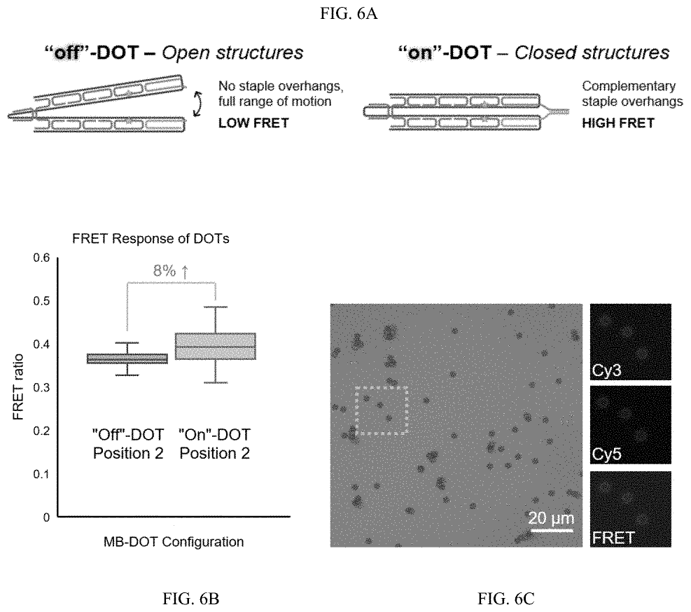

FIGS. 6A-6C are schematic diagrams illustrating an experiment in accordance with an embodiment of the invention, which investigates the position of reporters on the DNA nanosensors for maximum signal amplification. Generally, there is expected to be a maximum number and ideal placement of reporters on the DNA origami tweezer sensor, to increase the amount of molecular stacking signal-generation events upon analyte binding. For example, if three reporter sites are incorporated along the interior surface of the DNA origami tweezer arms, then one protein binding event would translate into stacking of six reporter molecules (four sites leads to eight reporters stacking, etc.). This approach can enable significant signal amplification upon cytokine binding to improve our limit of detection. To determine the maximum number and ideal placement of reporter positions in the sensor, approaches can be taken that are analogous to the work by Selnihhin et al (85), involving experimental calculations of the maximum intensity and difference in signal between freely open DNA-only tweezers (“off”-sensors) and closed structures (“on”-sensors) with varying numbers of reporter conjugation sites in the sensor (i.e., one though eight sites). The “on”-sensors can be prepared by modifying staples at the ends of the DOT arms to include a hybridized dsDNA duplex to prevent the DOTs from opening, yielding the highest possible signal; the “off”-sensors will not be end-modified to enable the full range of movement, yielding the lowest signal. FIGS. 6A-6C show experiments which take this approach. As shown in FIG. 6A, 10-nt DNA overhangs were installed at the ends of each arm with directly complementary sequences to assemble the DOT structures as dynamically open (“off”-DOTs) by omitting the overhangs and closed (“on-DOTs”) by including both staple overhangs. It is expected that the ideal chromophore position on the DNA helix will demonstrate the largest difference between “on”-DOTs and “off”-DOTs, with higher signal when the structure is “on.” Here, a pair of FRET reporters were used, conjugated in the interior of the DOT (Cy3 on one arm, Cy5 on the other). To characterize the FRET responses, a biotin tag was conjugated to the DOT hinge for immobilization on streptavidin-coated magnetic beads (MB-DOTs). These MB-DOTs were imaged in a glass microwell chamber on a fluorescence microscope (Zeiss LSM 700), using three acquisition channels for Cy3, Cy5, and FRET signals. The original position showed a 29% decrease in FRET signal upon DOT closure, indicating improper positions (data not shown). By simply repositioning the fluorophores (moved 4-nt along the interior helices), as shown in FIG. 6C, the response was improved to an 8% increase in signal. Further enhancements can be performed using thymine spacers in the hinge to minimize the current geometric constraint on the sensor's range of motion and enable a larger differential signal. The number of reporter pairs in the DOT can be maximized to yield the brightest signal with the highest SBR. FIG. 6A is a schematic diagram for the “off” and “on”-DOT sensors and expected FRET trends; FIG. 6B shows a summary of FRET analysis of MB-DOTs with updated fluorophore positions, the replicate ROIs for off- and on-DOTs are n=153 and 282, respectively. This position shows improved FRET trend with an 8% signal increase for the closed position, which indicates an excess constraint imparted by the tested hinge configuration. FIG. 6C shows an example of image data for the MB-DOT experiment showing three ROls-MBs with DOT coated on the surface.

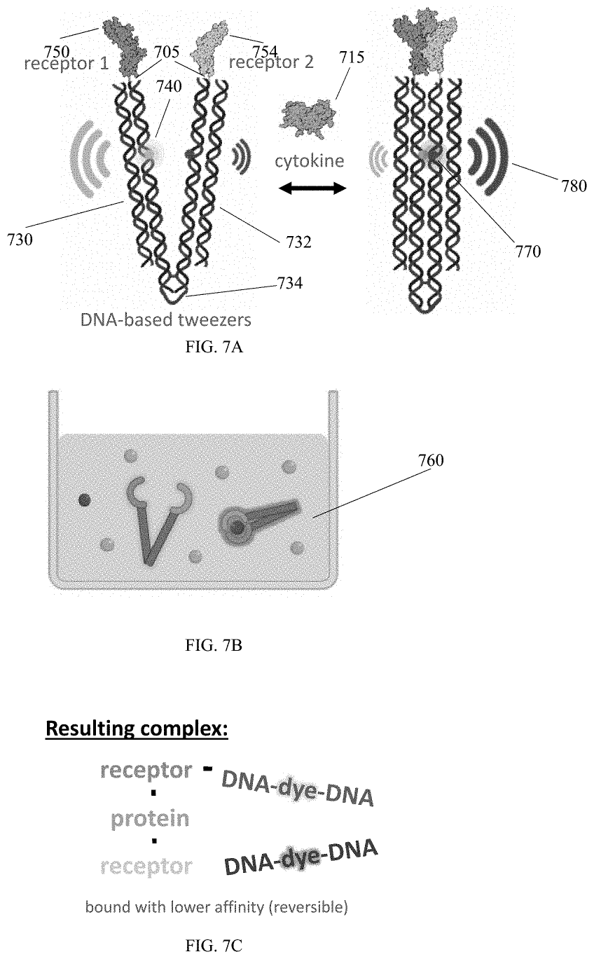

FIGS. 7A-7C are schematic diagrams illustrating operation of a DNA tweezer nanosensor, in an embodiment in accordance with the invention. FIG. 7A shows the nanosensor mechanism, involving opening and closing of the DNA tweezer in the absence or presence of a cytokine (respectively); FIG. 7B shows incubation of the sensors in whole blood, during which a dual-wavelength photoacoustic signal produced by the sensors is monitored over time; and FIG. 7C shows the resulting complex when the cytokine protein is bound to the receptors. In FIG. 7A, the DNA tweezer nanosensor includes a stabilized hinge 734 between each arm 730, 732 of the tweezer; at least one chromophore 740 in each arm; and a cytokine receptor 750, 754 in each arm. In the presence of a cytokine 715, there is a two-step binding process, in which the cytokine 715 binds to the first receptor 750 on the first arm 730 of the tweezer, and then the complex of the cytokine 715 with the first receptor 750 binds to the second receptor 754, thereby closing the hinge 734 of the tweezer. The closing of the hinge 734 structure, in turn, stacks 770 the chromophores of the two tweezer arms together, thereby increasing the photoacoustic signal 780 provided by the tweezer, by virtue of the increased photoacoustic signal released by the stacked 770 chromophores as opposed to the unstacked chromophores. Photoacoustic imaging enables whole blood 760 analysis by reducing the background signal levels, and offers deeper imaging depths. Reversible analyte complex formation enables continuous monitoring. Such sensors can enable bedside imaging with reduced background signal, and diminished need for sample processing. Further, such sensors can enable dynamic spatially resolved cytokine measurements in vivo.

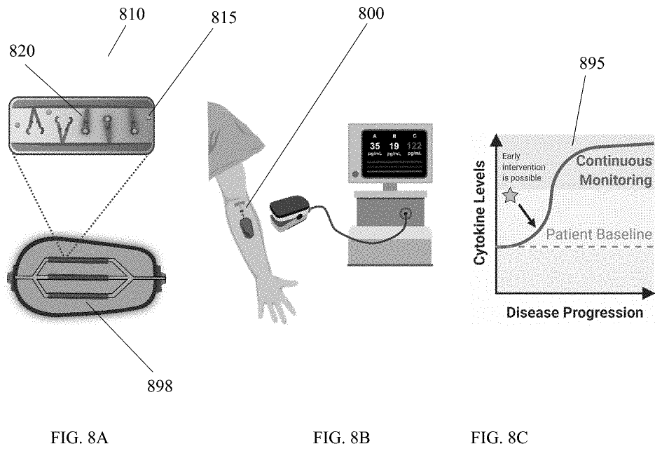

FIGS. 8A-8C are schematic diagrams illustrating a photoacoustic cytokine reporter system for point-of-care immune monitoring, in accordance with an embodiment of the invention. In FIG. 8A, photoacoustic cytokine nanosensors as taught herein, such as the DNA tweezers of FIGS. 4, 5A-5F, or 7A, are immobilized in fluid channels through which there flows the whole blood of a patient. The nanosensors detect cytokine levels in the whole blood, in real time, using the nanosensor operations taught herein. As shown in FIG. 8B, this enables bedside, in-line immune system monitoring, in which real-time cytokine levels detected in the nanosensor flow channel device are monitored. FIG. 8C shows a graphic display that illustrates a real-time analysis of patient-specific trends, which can be generated using cytokine levels measured by the nanosensor flow channel device. For example, continuous monitoring of cytokine levels can be performed. Early intervention can be enabled, such as when one or more cytokine levels rise above a patient baseline level. In addition to being immobilized in the fluid channels, nanosensors taught herein can also be injected, and can flow through the fluid channels for imaging. Further, nanosensors taught herein can also be injected without use of a device with fluid channels, either in an animal or in human point of care use, and can be imaged by a clinical photoacoustic imaging system.

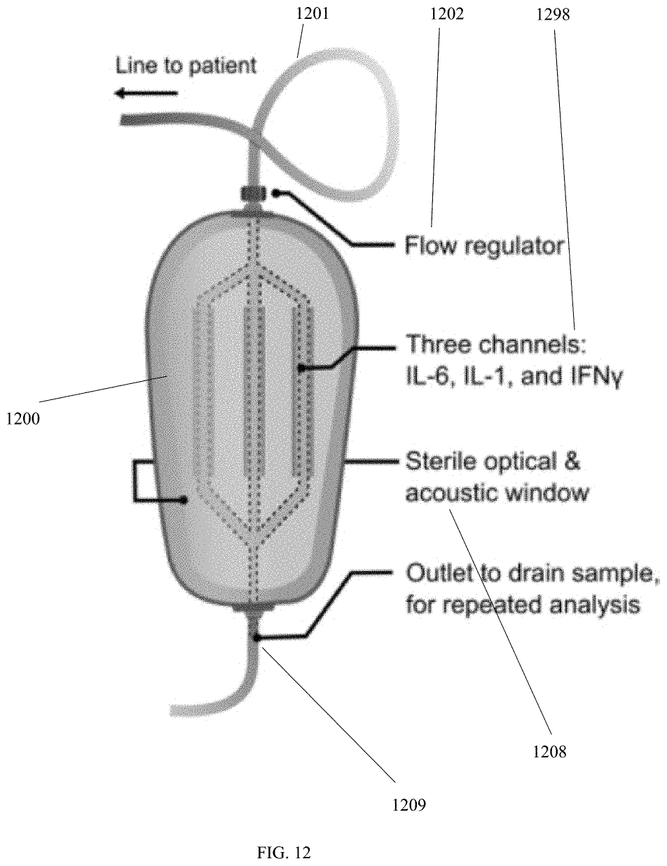

With reference to the embodiments of FIGS. 8A-8C, 7A-7C and others referred to herein, a system 810 for multiplexed photoacoustic detection of cytokines 715, 815 includes a plurality of nucleic acid-based nanosensors 820 each comprising at least two sensor arms 730, 732 (see FIG. 7A). Each arm of the at least two sensor arms 730, 732 comprises at least one photoacoustic reporter chromophore 740 and a cytokine receptor 750. Each arm 730, 732 is configured, in the presence of a detected cytokine in a fluid 760, such as whole blood, to induce stacking 770 of the at least one photoacoustic reporter chromophore 740 with at least one photoacoustic reporter chromophore in another one of the at least two sensor arms 730, 732, thereby increasing a photoacoustic signal 780 emitted by the nucleic acid based nanosensor 820, and each arm 730, 732 is further configured to induce unstacking of the at least one photoacoustic reporter chromophore 740 when the cytokine 715, 815 is not present, thereby decreasing the photoacoustic signal 780 emitted. Here, it is noted that the stacking of the at least one photoacoustic reporter chromophore 740 with at least one photoacoustic reporter chromophore in another one of the at least two sensor arms 730, 732 can, for example, include stacking of only one pair of photoacoustic reporter chromophores on two different sensor arms; and can include stacking of more than one pairs of photoacoustic reporter chromophores on two different sensor arms, with each stacked pair including a photoacoustic reporter chromophore on a different sensor arm. The plurality of nucleic-acid based nanosensors 820 (FIG. 8A) comprise cytokine receptors for at least two different types of cytokines. A photoacoustic measurement system 190 (see FIG. 1) is configured to detect the photoacoustic signals 780 (see FIG. 7A) emitted by the nucleic acid based nanosensors and to thereby permit distinguishing of a differential photoacoustic signal indicating a stacked or unstacked state of the at least one photoacoustic reporter chromophore of the nucleic acid based nanosensors, for each of the at least two different types of cytokines. The plurality of nucleic-acid based nanosensors 820 can be configured to induce the stacking of the at least one photoacoustic reporter chromophore through a two-step binding process including a first binding of a first cytokine receptor 750 (FIG. 7A) in a first sensor arm 730 with the detected cytokine 715, and a second binding of a second cytokine receptor 754 in a second sensor arm 732 with the complex of the first cytokine receptor 750 and the detected cytokine 715. The nucleic-acid based nanosensors can be configured to detect two or more of: IFNγ, IL-6, and IL-1. The nucleic-acid based nanosensors can include at least two of the following pairs of cytokine receptor pairs, one cytokine receptor 750 of each pair being conjugated on a first arm 730 of the at least two sensor arms, and another cytokine receptor 754 of each pair being conjugated on a second arm 732 of the at least two sensor arms, the cytokine receptor pairs comprising: IFNγR1 and IFNγR2; IL-6R and gp130; and IL-1R1 and IL-1RAcP. The system can be configured to perform continuous real time monitoring 895 (see FIG. 8C) of levels of the at least two different types of cytokines in the fluid. The nucleic-acid based nanosensors can include DNA origami based nanosensors (such as those of the embodiment of FIG. 4); and can include protective shielding from biological conditions, such as PEGylation and others taught herein. The nucleic-acid based nanosensors can each include a tweezer structure (see FIG. 7A) that includes two arms 730, 732 attached at a central hinge 734 configured to perform the inducing of the stacking in the presence of the detected cytokine 715, and the inducing of the unstacking when the cytokine is not present, and can include modification sites 705 upon which the cytokine receptors 750, 754 and the at least one photoacoustic reporter chromophores 740 are installed. The nucleic-acid based nanosensors may further comprise a single stranded DNA tether (see 507 in FIG. 5F) between the two arms of the tweezer structure. The at least one photoacoustic reporter chromophore 740 (see FIG. 7A) can include at least one of an indocyanine green analog, a phthalocyanine chromophore, and a BODIPY analog. The at least one photoacoustic reporter chromophore 740 can further include a molecular scaffold. The molecular scaffold can, for example, be or include a polycyclic molecular scaffold, and in one example can be a xanthene molecular scaffold. The system can be further configured to provide a fluid flow connection to an intravenous device 800 (see FIG. 8B) configured to inject the plurality of nucleic acid-based nanosensors 820 into the fluid. The plurality of nucleic acid-based nanosensors can include at least part of an injectable agent configured to permit spatially resolved imaging of at least one cytokine in a living subject, and can, for example, permit spatially resolved imaging of at least two different types of cytokines in a living subject. The system can further include a plurality of fluid channels 898 configured to flow the fluid including the plurality of cytokines, the plurality of nucleic acid-based nanosensors being inside at least one fluid channel 898 of the plurality of fluid channels, the system being configured to permit photoacoustic measurement of the fluid in the at least one fluid channel. The plurality of nucleic acid-based nanosensors can be immobilized inside the at least one fluid channel 898 of the plurality of fluid channels, or may be flowing in injected solution inside the at least one fluid channel of the plurality of fluid channels 898. A device such as that of FIG. 12 can, for example, be used. The system can further include a cassette 1200 (see FIG. 12) comprising the plurality of fluid channels 898 and the plurality of nucleic acid-based nanosensors.

Adaptation of DNA Origami Tweezer Nanostructure for IFNγ, IL-6, and IL-1 Detection

The DNA origami tweezer nanostructure, in accordance with an embodiment of the invention, can be functionalized by conjugating IFNγR1 on the first arm and IFNγR2 on the second arm. When IFNγ binds to IFNγR1, the IFNγ-IFNγR1 complex can then bind to IFNγR2 on the other arm, facilitating closure of the DOT and stacking of the reporters to generate an orientation-dependent PA signal correlated with increasing analyte concentration. This detection mechanism can be analogous to that of the sensor of reference (7). This two-step binding mechanism is well characterized in physiological conditions, and IFNγR2 can only bind IFNγ when the cytokine is complexed with IFNγR1. In this case, a conformational change enables the IFNγ-IFNγR1 complex to heterodimerize with IFNγR2 to form the IFNγ-IFNγR1-IFNγR2 complex (95). The sensor platform can be adapted for detecting many other cytokines by simply changing the recognition elements, such as for IL-6 and IL-1 detection. In cell-cell communication processes, soluble IL-6 engages with its primary receptor, IL-6R, creating an IL-6-IL-6R complex that can then associate with the protein gp130. This heterotrimeric complex subsequently dimerizes and initiates intracellular signaling via the JAK/STAT pathway (96). Similarly, IL-1 participates in signaling through binding to IL-1R1, creating a complex that can then bind to IL-1RAcP (96, 97). Like IFNγR2, the gp130 and IL-1RAcP proteins only have affinity for preformed ligand-primary receptor complexes (98, 97). IFNγR1, IFNγR2, IL-6R, gp130, IL-1R1, and IL-1RAcP, each with a C-terminal His-tag, are commercially available (Sino Biological). The C-termini of each receptor pair is closely oriented upon complex formation, suggesting that a single covalent modification at this site is ideal for sensor design in promoting device closure upon receptor complex formation. To achieve this modification, a single-site covalent conjugation strategy can be used, such as with the NBzM linker (99) or DNA-templated protein conjugation (100). After covalent attachment of each receptor of the cytokine pairs to unique ssDNA staple strands, the conjugates can be hybridized onto the DOT that was pre-annealed using a temperature ramp in a 1× TE/Mg2+ buffer.

Protection Strategies for DNA Origami Tweezer Nanosensors

Protection strategies can be deployed to shield the DNA nanosensors, such as DNA origami tweezer nanostructures, in accordance with an embodiment of the invention. Examples of protection strategies include, but are not limited to: modification of staple strands with various forms of PEG (105, 106), coating the surface with protein or polymer (107, 108), and/or insertion of UV-initiated (109) or click-reaction crosslinks (110).

Use of Indocyanine Green (ICG) Analogs for Photoacoustic Imaging

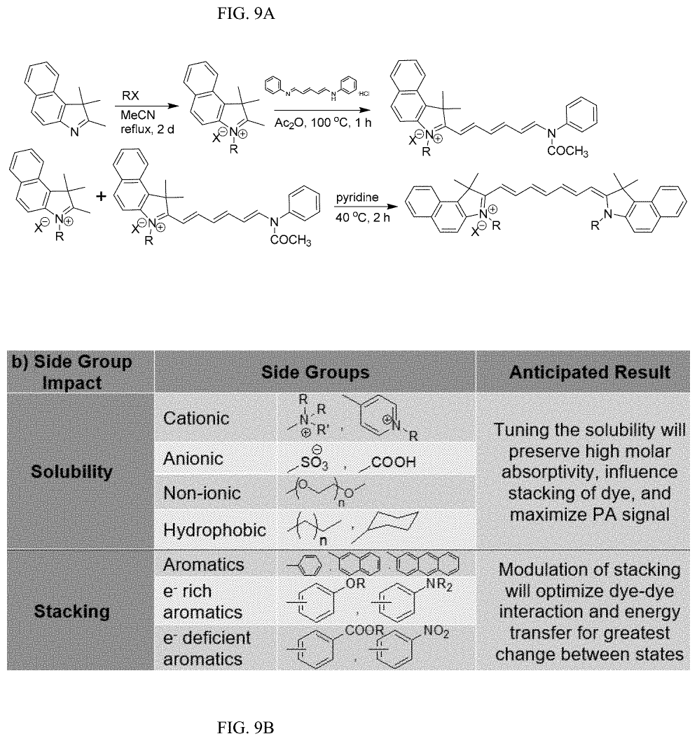

In accordance with an embodiment of the invention, an analog of indocyanine green (ICG) can be used to maximize the sensitivity of the DNA origami tweezer for cytokine detection. ICG is FDA-approved for in vivo use and widely used for both fluorescence and PAI (111). ICG is a hydrophilic tricarbocyanine dye with peak NIR absorption and moderate fluorescence QY (˜14%). Studies have shown that J-aggregated ICG exhibits significantly more photothermal stability than monomeric or dimeric ICG (112). Additionally, J-aggregated ICG absorbs maximally at approximately 890 nm, whereas monomeric and dimeric ICG have barely any absorbance at 890 nm (113, 114). With ICG, there is a large spectral shift (100 nm from monomer to J-aggregate, and 200 nm from dimer to J-aggregate) that can be exploited as a highly sensitive and specific readout of reporter conformation (and therefore cytokine binding). As few as four ICG molecules are sufficient to induce J-aggregation, provided that the four molecules are properly aligned (115). For PAI, signal is generally strengthened when greater numbers of contrast agents are simultaneously interrogated (116, 117). Rational design of a molecular scaffold bearing multiple ICGs will optimize ICG-ICG interactions and improve PA signal. Tetramerized ICGs will selectively signal DOT closure and binding events via PA at an excitation wavelength of ˜890 nm, yielding a highly sensitive readout. Commercially available ICG analogs can be obtained with single side chain modifications for attachment to the DNA; alternatively, ICG analogs can be synthesized. FIG. 9A is a schematic diagram of a typical scheme for ICG synthesis, which can be used to form the basis of synthesis of ICG for use in a nanosensor in accordance with an embodiment of the invention. FIG. 9B is a schematic diagram showing how the structure of ICG can be functionalized with numerous groups to tune its solubility and stacking properties. In FIG. 9B, ICG is a carbocyanine dye with R=R′=butane sulfonate.

Xanthene Molecular Scaffold

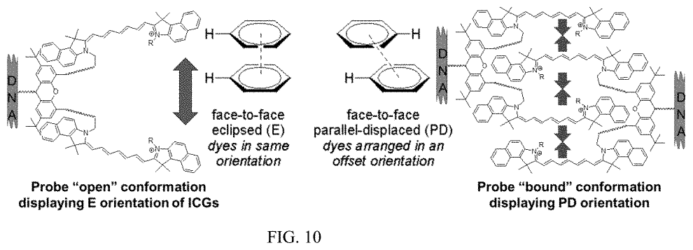

In accordance with embodiments of the invention, a xanthene molecular scaffold can be incorporated to increase ICG concentration and control its orientation for stacking, in order to significantly increase cytokine sensor sensitivity. It will be appreciated that other molecular scaffolds can be used, including polycyclic molecular scaffolds. In an embodiment, initially, the two ICG molecules affixed to the molecular scaffold (xICG₂) would interact in the energetically least-preferred, highest energy “eclipsed” state; upon probe closure, a total of four ICG molecules would be brought together in the thermodynamically preferred parallel-displaced geometry (119). This dramatic change in energy and the co-localization of four ICG molecules will result in significantly increased PA signal characteristic of J-aggregated ICG. The xanthene scaffold (FIG. 10) is adapted from established chemistry (120) and is effective in supramolecular coordination (121). FIG. 10 shows orientations of xanthene scaffold-conjugated ICG in both “open” and “bound” probe conformations, which can be used in a DNA nanosensor in accordance with an embodiment of the invention. Dispersive forces in the eclipsed (E) state repel dye bound on the same xanthene molecule (left side of FIG. 10). Attractive forces in the parallel-displaced (PD) state allow for thermodynamically favorable stacking (right side of FIG. 10). Use of the xanthene ring structure effectively doubles the concentration of dye—thereby increasing PA signal—and coordinates the dye orientation. Starting from xanthone, the ketone is reduced to a hydroxyl group. Conjugation to DNA occurs at this central position; sequential bromination and carboxylation functionalize the opposite side of the ring system to allow for conjugation to the dye.

Attachment of the ICG reporters, whether directly or via the xanthene scaffold, to the DOT sensor can use established conjugation chemistry (azide/alkyne click chemistry, amine/NHS ester, thiol/maleimide, etc.). For example, the conjugatable element for ICG can be an alkyne group due to synthetic feasibility and compatibility with various conjugation chemistry reactions. Williamson etherification of the 9-hydroxyl xanthene scaffold with 4-bromo-butyne yields alkyne-modified xanthene. This scaffold can then be conjugated to the azide-functionalized DNA using the same methodology as for conjugation of the alkyne-modified ICG directly to the DNA probe.

Immobilization of DNA-Based Sensors Inside Sample-Contact Tubing

In accordance with an embodiment of the invention, multiplexed DNA-based probes taught herein can be immobilized into separate wells of a fabricated cassette and be deployed for inline PA cytokine imaging. The system can be used in rapid, multiplexed, bedside analysis of cytokines, for example through remote imaging with an inline and removable system. This is advantageous as it enables the user to access blood samples directly from the patient and perform the multi-cytokine measurement all in one device, with results reported in real time to obtain valuable trend information.

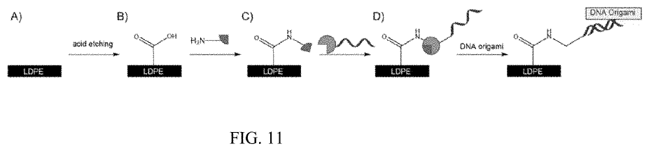

To enable repeated measurement of whole blood samples using the same fabricated device, the DNA origami sensors can be covalently immobilized inside sample tubing. Polyethylene tubing (LDPE, BTPE-50) can be used to house the samples for analysis. To immobilize the DNA origami on the interior surface of the polyethylene tubing, a click-chemistry approach can be used after preparation of a reactive surface with the example scheme shown in FIG. 11, which is a schematic diagram illustrating immobilization of DNA origami on surface of polyethylene tubing, in accordance with an embodiment of the invention. First, in step (A) of FIG. 11, the interior surface of LDPE tubes can be chemically etched to incorporate reactive carboxylic acid groups, e.g., by treatment with chromic acid or other oxidants (124, 125). The LDPE-COOH surface can be further modified through incubation of amine-containing substrates in aqueous buffer (126) to install a variety of click-chemistry handles, based on the choice of linker (amine-PEG4-alkyne, ethylenediamine, etc.), as in step (B) of FIG. 11. Once a click-chemistry reactive group (thiol, alkyne, etc.) is installed on the LDPE surface, an attachment ssDNA staple strand modified with the complementary reactive group (maleimide, azide, etc.) can be introduced to coat the surface in the ssDNA staple, as in step (C) of FIG. 11. Hybridization of DNA origami sensors to the surface of LDPE, as in step (D) of FIG. 11, can be achieved through heated incubation of the sensor facilitated by a complementary staple overhang, thereby resulting in immobilized DNA-based sensor on the tubing, as in step (E) of FIG. 11.

Fabrication of a Cassette for an Inline Cytokine Detection Device

FIG. 12 is a schematic diagram of an inline cassette device for cytokine detection, in accordance with an embodiment of the invention. Features include inline attachment with sample outlet for repeated sampling, three channels with immobilized sensors, and an optical and acoustic window for PAI measurement. Interior surface-modified LDPE tubes can be incorporated into a three-path fluidic channel, housed inside a cartridge comprised of optical and acoustic-permissive medium such as PVCP (127). The cassette will be connected to IV tubing that will enable direct patient access for a whole blood sample, a flow regulator to restrict blood removal during analysis, and drainage to remove the sample from the cassette after sample imaging. Atop the cassette is a sterile acoustic and optical window (Tegaderm film) to ensure longitudinal inline monitoring without sterility issues. For PA imaging, acoustic gel can be safely applied on top of the device for contact with the instrument probe. Simultaneous photoacoustic imaging of the three channels can be used with the inline device. The three channels can, for example, be at least 500 μm wide (minimum size) to ensure the channel diameter is at least 2× larger (Nyquist criterion) than the lateral resolution of a 20 MHz linear array transducer (250 μm) that can be used with the FujiFILM Visualsonics system. The width of the inline device can, for example, be less than 2 cm (limited by the imaging field of view by the transducer). The device design is a trade-off between the dimensions of the channel, amount of blood required to fill the channels, and the sensitivity of the imaging system to detect the cytokines within that given volume. The wider the channel, the higher the photoacoustic signal due to greater volume of blood or sensor in the channel. Preliminary work on imaging blood with various tube widths (i.e., mimicking a channel) showed that a 500 μm diameter tube yields a good PA signal with the four-arm sensor taught herein (reference 7). For a 500 μm wide and 2 cm long channel, we will need 5 μL of blood/channel, i.e., a total of 15 μL of blood for the three channels.

In accordance with an embodiment of the invention, the inline cassette device of FIG. 12 can, for example, include: a line to the patient 1201; a flow regulator 1202; three channels 1298, which can, for example, be used to image each of three cytokines, such as IFNγ, IL-6, and IL-1; a sterile optical and acoustic window 1208; and an outlet 1209 to drain sample for repeated analysis. The fluid channels 1298 can, for example, be part of a cassette 1200 which can be installed and removed from the device. The device can be used to monitor the dynamic changes of more than one cytokine, such as IFNγ, IL-6, and IL-1. The device can be configured to permit detection of the photoacoustic signals emitted by the nucleic acid based nanosensors by a photoacoustic measurement system and to thereby permit distinguishing of a differential photoacoustic signal indicating a stacked or unstacked state of the at least one photoacoustic reporter chromophore of the nucleic acid based nanosensors, for each of the at least two different types of cytokines. For example, the device can include an optical and acoustic window for PAI measurement, such as a sterile optical and acoustic window 1208.

Spatial Cytokine Signaling for Monitoring Response to Immunotherapy

In another embodiment according to the invention, spatial cytokine signaling can be used for monitoring response to immunotherapy. FIG. 18 is a schematic diagram showing an overview of use of nucleic-acid based sensors for measuring cytokines through photoacoustic imaging in vivo, in accordance with an embodiment of the invention. Tracking real-time dynamics of cytokines can, for example, enable research into predictive models for response to therapy in RA patients, leading to more targeted treatment approaches. As shown in FIG. 18, cytokines are key mediators of inflammation, such as in a rheumatoid arthritis (RA) joint. Using a DNA-based cytokine sensor platform in accordance with embodiments of the invention, cytokine measurements can be performed in vivo using photoacoustic imaging. Sensors taught herein can, for example, be grafted onto a hydrogel substance as a bio-compatible carrier. A mouse model of RA can be used to induce localized increases in cytokine production. Sensors can be injected directly into RA-affected joints and PA imaging can be used to measure in vivo levels of cytokines, such as IFNγ, IL-6, and IL-1.

For many conditions, systemic measurements may have less scientific value than localized diagnostics. New therapeutic approaches are being developed to control the activity of cytokines for a variety of autoinflammatory conditions, psychiatric disorders, infectious diseases, and cancers (B3, B4, B20, B21). Yet, it is difficult to evaluate the impact of systemic treatment approaches on the regulation of localized cytokine activity. The cytokines that are typically measured in a blood sample are those that have not been consumed at local sites or rapidly removed from circulation (B22). Often, the levels of these circulating cytokines in either healthy or diseased individuals are below the limit of detection achievable by commercially available assay kits (B19). Individual blood samples are also susceptible to impacts of preanalytical factors such as physical activity, food intake, drugs, sample collection and handling, as well as analytical aspects including assay methods, reagents, and standards used (B19). In contrast, localized measurements could give direct feedback regarding the condition of a specific area, as well as on the impact of therapeutic intervention directly at the site of interest, such as intracerebral hemorrhage in the brain or a specific joint for osteoarthritis (B23). Local measurements may also be present in large enough concentrations to be predictive of a disease state, even when systemic measurements of the cytokines are not. For example, even though plasma cytokine levels were not found to be predictive factors for relapse, an elevated IL-8 level in the local rectal mucosa was found to be an independent significant risk factor for future relapse in patients with quiescent ulcerative colitis (B24). Systemic cytokine levels may be good indicators of systemic inflammation, but are nonspecific and not useful for localized assessments.

In an embodiment of the invention, photoacoustic cytokine reporter (PACyR) imaging agents taught herein can be used to perform simultaneous monitoring of multiple cytokines to monitor localized cytokine dynamics in vivo.

In one embodiment, BODIPY analogs can be used as one or more photoacoustic reporter chromophore with nucleic acid based nanosensors taught herein. Attachment of the dye, whether directly or via the xanthene scaffold, to the DNA probe can use established conjugation chemistry (azide/alkyne click chemistry, amine/NHS ester, thiol/maleimide, etc.). A conjugatable element for the aza-BODIPY can be an alkyne group. Williamson etherification of the 9-hydroxyl xanthene scaffold with 4-bromo-butyne yields alkyne-modified xanthene. This scaffold can then be conjugated to the azide functionalized DNA using the same methodology as for conjugation of the alkyne-modified dye directly to the DNA probe. Purification can be performed via HPLC with characterization by mass spectrometry to ensure successful conjugation.

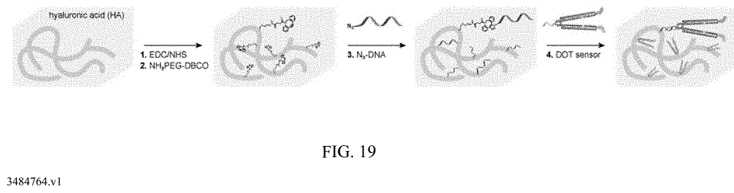

In another embodiment, in order to stabilize the imaging probe for diffusion from the site of injection, nucleic-acid based probes taught herein can be immobilized, equipped with nanosensors taught herein, into a hydrogel. The device can be deployed for in vivo synovial cytokine imaging of the localized inflammation characteristic of rheumatoid arthritis (RA), or other localized condition, either in animal research or in human point of care use. An injectable sensor system can be used for spatially resolved cytokine measurement by photoacoustic imaging. A biocompatible sensor-coated injectable hydrogel can, for example, be used with nanosensors taught herein. The nanosensors can remain in an injected joint space in a biocompatible matrix that can withstand degradation. Recently, Chen et al demonstrated the remarkable performance of a DNA-grafted hyaluronic acid (HA) gel for intra-articular delivery of a spherical nucleic acid therapeutic in an osteoarthritis mouse model (B107). A similar approach can be taken, in generating a click chemistry-compatible HA gel through which DOT cytokine sensors can be immobilized, as shown in FIG. 19, in accordance with an embodiment of the invention. Briefly, for example, HA is dissolved and subjected to an EDC-NHS reaction to install a reactive amine group in the HA surface, followed by treatment with a linker (NH₂-PEG4-DBCO) to incorporate a click-reactive surface. An azide-modified oligonucleotide attachment strand is reacted with the accessible DBCO to generate a covalent DNA-HA gel. The complementary attachment strand is incorporated into the respective cytokine-DOT structures during annealing, such that the sensors can be captured on the DNA-HA surface through incubation. The result can be an injectable sensor system capable of measuring multiple separate cytokines (for example, IFNγ, IL-6, and IL-1), in a living animal or human subject, in accordance with an embodiment of the invention. Further, this can, for example, create an injectable agent configured to permit spatially resolved imaging of at least one cytokine in a living subject, in accordance with an embodiment of the invention.

Interferon Gamma DNA Nanosensor

Tracking protein levels in the body is vital in both research and medicine, where understanding their physiological roles provides insight into their regulation in homeostasis and diseases. In medicine, protein levels are actively sampled since they continuously fluctuate, reflecting the status of biological systems and provide insight into patient health.

Tools for quantitative, real-time monitoring of proteins are necessary for applications ranging from basic research to medical monitoring (A1), where tracking these targets is critical in elucidating biochemical pathways and disease progression (A2). In medicine, monitoring protein levels aids in determining treatment options for patients and is important in checking patient health during treatments, such as dialysis (A3) and intensive care (A4). One such protein is interferon gamma (IFNγ), a clinically relevant protein with immunoregulatory functions that play critical roles against infection. IFNγ is an active immune system regulator (A5) approved by the FDA as a biologic drug (A6). IFNγ is a small immune system signaling protein, active in both innate and acquired immunological functions, including adaptive immune response and early host reaction to infection (A5). The dysregulation of endogenous IFNγ production has been used as a biomarker for autoinflammatory and autoimmune diseases, tuberculosis, and cancer, among others (A7-A10). IFNγ regulates the expression of many other cytokines, observable within hours, with potential implications toward the progression of diseases and injuries downstream on longer time scales of days or weeks (A11-A16). This time frame emphasizes the clinical relevance of temporally resolved quantification of IFNγ toward the diagnosis and treatment of various diseases, to determine therapeutic windows for intervention.

Moreover, IFNγ is used as a drug for the treatment of chronic granulomatous disease (A17) and has shown antitumor properties for cancer immunotherapy (A18). However, IFNγ therapy is not without risks, requiring patient-specific dosages that must be precisely controlled (A19), (A20) since elevated levels are harmful and associated with major side effects (A21). Moreover, recent studies have shown that IFNγ can promote tumor growth under specific conditions (A22), (A23). Thus, continuous monitoring of the IFNγ protein level is necessary to maintain a safe therapeutic dosage in patient-specific treatment and to prevent the associated side effects.

Several techniques have been developed to monitor protein levels for clinical applications. The clinical gold standard for protein quantification is enzyme-linked immunosorbent assays (ELISA), a highly sensitive method that uses antibodies to capture and quantify proteins, like cytokines (e.g., IFNγ), but suffers from slow throughput, making it undesirable for rapid detection of proteins (A24). Alternatively, electrochemical sensors provide simple, low-cost, and rapid detection of proteins by using targeting receptors that produce redox signals upon binding (A25), (A26). These methods provide single point measurements with high precision. However, in measuring the dynamic concentration of cytokines, these techniques require successive sampling, which causes cytokine levels to fluctuate, making them unreliable in measuring changes in cytokine levels (A27). Since cytokines are associated with the immune response of the body, blood collection methodology and sample age, among other factors, can drastically affect the measured cytokine levels (A28-A30). Thus, dynamic cytokine levels are challenging to measure in real-time using these methods. Successful translation of these tools in vivo with microfluidics and biochips has also been proven difficult due to poor sample recovery across dialysis membranes and irreversible binding, respectively (A31). Recently, the development of aptamers with fast response times and moderate affinity provide an avenue for reversible recognition elements. However, they are only available for select targets due to their complex development process (A32). Although sampling-based methods have high sensitivity, their translation for continuous in vivo monitoring is challenging.

For systemic conditions, the most clinically relevant protein levels are typically in the bloodstream, and for conditions with localized inflammation such as rheumatoid arthritis, the local cytokine signaling in synovial tissue is more clinically relevant. Considerations like these make sampling a significant challenge toward continuous in vivo monitoring. Tissue interference limits sensing capabilities against noninvasive optical-based techniques. Although fluorescent methods are popular for continuous monitoring in vivo, they are limited to imaging depths of a few millimeters and are susceptible to light scattering in tissue (A33).

Photoacoustic (PA) imaging has been growing in popularity as a bioanalytical technique for in vivo measurements. PA is a hybrid imaging method that uses the PA effect, a physical event that converts optical energy into acoustic waves, to provide structural and functional information with microscale resolution (A34). This method increases the tissue depth resolution of optical techniques since sound has a higher tissue penetration depth and little scattering in comparison to light (A35), (A36). PA uses a combination of current optical and ultrasound methods to detect optical absorption from photoactive contrast agents. Currently, PA probes (A37-A39) and sensors (A39-A42) for ions and small molecules allow measurements of up to 5 cm into tissue, targeting ligands of interest, and activation in response to biological processes (A43), making PA based sensing an ideal option for monitoring protein levels in vivo.

Developing PA-based sensors for in vivo measurements must meet dependable sensor performance, even at the in vitro stage (A44), including turn-on signal, small size, good selectivity, and reversibility (A45). “Turn-on” sensors are preferable to “turn-off” sensors since a signal increase is more reliable than intensity loss, resulting in a reduced background signal from nonspecific interactions (A46). In particular, the dye phthalocyanine (Pc) may be a good probe for such applications. Pc dyes have increased optical absorbance and PA signal upon aggregation or stacking (in J-aggregate configuration) of the molecules, and they have well-studied, tunable PA properties (A47). The aggregation-induced PA signal change can be taken advantage of to design a “turn-on” sensor, using the stacking and unstacking of a pair of Pc dyes as a reversible PA reporting element.

In addition to “turn-on” signaling, sensors need to be both selective and reversible as well. Selectivity toward the protein of interest prevents interfering species in the biological matrix from compromising the response of the sensor. Sensor reversibility is also necessary to actively track changes in protein levels over time. One of the major hurdles in continuous protein detection is finding a selective and reversible recognition element. Benchtop methods utilize antibodies for recognition due to their high affinity; however, this renders the methods nearly irreversible (A48). Using enzymes for recognition is not applicable since the only known enzyme for IFNγ is a nonspecific protease with no measurable side products (A49). Alternatively, native receptors are attractive recognition elements for real-time tracking of analytes due to their reversible affinity (A50) and predictable selectivity (A51).

Combining PA-based reporting and IFNγ receptor-based recognition requires an architecture to exploit these elements. DNA has been popularly used for making sensor architectures or nanomachines (A52). In particular, linear DNA architectures are a common nanomachine motif, defined as a V-shaped gate, opening and closing based on hybridization (A53). Often in these structures, Förster resonance energy transfer (FRET) is used to indicate conformational change because the signal is distance dependent. Due to similar conditions required for Pc dye stacking, the simple DNA structure is an ideal vehicle for producing the mechanism required to stack and unstack Pc dyes (A54). Receptor binding in this structure offers a reversible solution to potentially drive the structures toward open and closed states for Pc dye stacking.

In one embodiment, we demonstrate a proof-of-concept nanosensor for protein detection with a goal toward future in vivo use by combining (a) a Pc-derived PA dye as a “turn-on” reporter, (b) a DNA-based nanostructure scaffold to facilitate Pc dye stacking, and (c) IFNγ-specific receptors for reversible recognition. Here, we create a PA, DNA-based nanosensor for the detection of IFNγ. Our design uses receptor binding to drive the stacking of Pc dyes, resulting in an increase of PA signal. Together, these components provide PA amplification and protein detection.

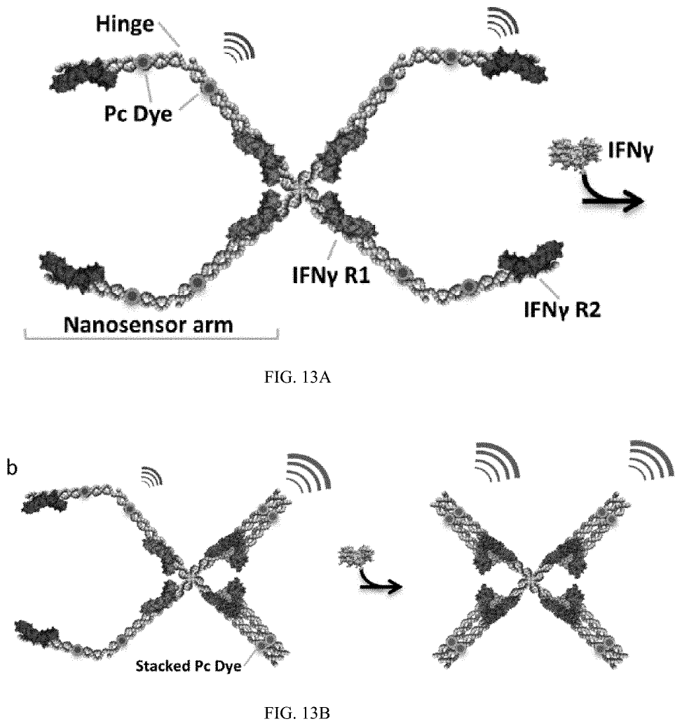

In one embodiment, a nanosensor design incorporates four arm-like binding regions connected by a central intersection, where each “arm” is a self-contained sensor containing dyes and receptors, as shown in FIGS. 13A and 13B. FIGS. 13A and 13B are schematic representations of a four-arm nanosensor, which is an example of another type of nanosensor that can be used as a nanosensor in accordance with an embodiment of the invention. FIG. 13A shows the nanosensor with its arms freely moving in solution when no analyte is present. FIG. 13B shows that, upon analyte binding, the arms fold in, and the dyes are held in close proximity, causing a change in PA signal. We use receptor-mediated binding by attaching receptors specific for IFNγ on each end of the arm to initiate closure. The active part of the sensor uses a two-step recognition system, designed to open and close the complex in response to IFNγ binding. We utilize IFNγ receptor 1 (IFNγR1) and receptor 2 (IFNγR2) as a two-step recognition element for the sensor. IFNγR1 is specific for IFNγ and undergoes a conformational change when bound (step 1); whereas IFNγR2 has no affinity for IFNγ and only binds to the IFNγR1-IFNγ complex, resulting in heterodimerization into INFγR2-IFNγR1-IFNγ (step 2) (A55-A57). Sensor response arises when a conformational change at the hinge brings the Pc dyes in close proximity to each other, in response to IFNγ binding. To minimize sensor-to-sensor interactions, we placed IFNγR2 on the outermost portion of each of the four arms and positioned IFNγR1 near the center, hindering access from other sensor arms. This design works with the sequential nature of the receptor binding to maximize the chance of intrasensor interactions and to minimize sensor-to-sensor cross-linking. However, this does not completely eliminate the possibility of cross-linking between arms from the same or different nanosensor. In addition, the four-arm architecture increases the probability for IFNγ-binding to cause arm bending and allow Pc dye stacking for change in PA signal, as opposed to free individual arms binding to one another resulting in an open configuration, inhibiting dye stacking and thus no change in PA signal. This design also increases the localized concentration of the Pc dye molecules, amplifying the PA signal achieved from a single nanosensor.

The hinge of the four-arm nanosensor uses a ssDNA motif with a repetitive sequence containing a single nucleotide (TTTT) to hinder interaction between bases with the same polarity. This motif is a simple and flexible single-stranded sequence, widely used as a bending point in nanomachines, molecular probes, and DNA hybridization (A58). This allows the opening and closing of the sensor arms to depend on the receptor-target interaction without requiring any interactions on the hinge. All arms contain the same structure comprised of five DNA strands; one long strand that serves as the foundation, and four short strands that bind to the foundation. At the center of the foundation, a 4-base long hinge is located that is designed not to bind any part of the shorter strands. Two of the short strands are functionalized with the Pc dye and located adjacent to the hinge. The other two strands are functionalized with the IFNγ receptors and immediately flank the dye-modified strands. The IFNγR1-modified strand is located at the proximal end, while the IFNγR2-modified strand is located at the distal end, relative to the overall nanostructure. The long foundation strand also has a short sequence that binds to the central intersection, connecting the arms together to form the four-arm complex seen in FIGS. 13A and 13B.

We used a combination of gel electrophoresis and transmission electron microscopy (TEM) to assess the nanosensor's structural characteristics. To determine if the DNA sequences assemble into a larger structure, we utilized polyacrylamide gel electrophoresis (PAGE). PAGE methods produce the best separation of DNA structures by size and provide insight into the quality of the folded structures by the clarity of the gel band (A59). Gel electrophoresis was carried out in 20% polyacrylamide gel using 0.5× Tris-borate-EDTA buffer at 70 V for 1 h. Gels were stained in 0.5 μg/mL ethidium bromide for 10 min and code blue protein stained before imaging. A gel imager (ChemiDoc XRS+System, BioRad) was used for imaging. Here, we expect an increase in band height, as the DNA structures progress from the smallest constituent (center piece) to a single arm and then the entire DNA structure, since larger structures migrate slower in electrophoresis gels. Our results show high molecular weight bands in the gel for each sensor component and full sensors as well as smeared bands indicative of a range of misfolded secondary structures migrating at 200 bp. The gel band of the complete sensor (with receptors) migrates at approximately 1500 bp (single-stranded DNA ladder), which is substantially larger than the total amount of DNA bases in the structure (668 bp). This slower migration pattern is expected for double-stranded and 3D structures; single-stranded DNA migrates at its expected bp content, double-stranded DNA migrates at a slower rate, and the presence of additional secondary structures (DNA motifs, hairpins, and circular DNA) further restricts migration (A60). This result supports the formation of larger DNA structures (whole sensor with associated proteins in the full DNA scaffold and predicted 4-arm structure) from the individual sensor components and is further supported by TEM.

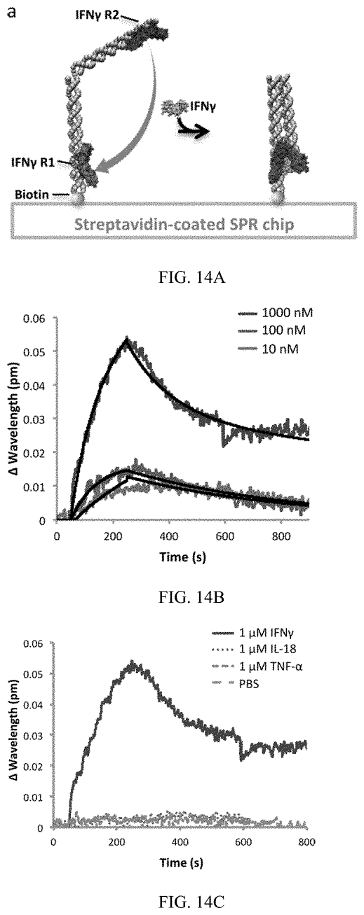

We determined the nanosensor's in situ binding kinetics and selectivity with surface plasmon resonance spectroscopy (SPR). SPR is a label-free technique for real-time quantification of ligand-binding affinities and kinetics in a native environment. SPR provides qualitative and quantitative information over the selectivity and specificity of diverse interactions between ligands, proteins, drugs, viruses, and cells (A61). Real-time kinetic analysis of macromolecular interactions is essential to determine the type of binding kinetics of ligand-receptor interactions. FIG. 14A is a schematic diagram of the nanosensor two-step binding mechanism between IFNγ and its receptors, in an experiment in accordance with an embodiment of the invention. We immobilize a single sensor arm on an SPR chip by biotin-streptavidin interaction. (Step 1) IFNγ binds to IFNγR1, forming the IFNγR1-IFNγ complex. (Step 2) The IFNγR1-IFNγ complex opens a binding site for IFNγR2, resulting in the closure of the sensor arm upon the formation of the IFNγR2-IFNγR1-IFNγ complex. FIG. 14B is a sensogram graph for IFNγ binding to the immobilized nanosensor, in an experiment in accordance with an embodiment of the invention. The lines are the response at three different concentrations overlaid with the kinetic fit of a 1:2 interaction model. FIG. 14C is a graph showing that the nanosensors are selective toward IFNγ compared to TNFα, IL-18, and PBS, in an experiment in accordance with an embodiment of the invention.

In the experiment of FIGS. 14A-14C, to immobilize the sensors, biotin-terminated single arms of the sensor were bound to a streptavidin-coated SPR chip. As IFNγ in solution binds to the sensor and the arm closes (FIG. 14A), changes in mass on the surface result in an increase in signal on the sensogram. We fit the SPR binding curves to different kinetic models to determine the best fitting model using a Chi-squared (χ²)<1 for each plot for evaluation (A62). A 1:1 binding fit is indicative of one analyte binding to one receptor, and a 1:2 fit is one analyte to two receptors, as expected for the nanosensor arm. The sensograms demonstrate binding between the arms and respond dynamically to injections of 0.1-1000 nM IFNγ (1.7 ng/mL-17 μg/mL). We found the best fit, χ²<1, is a 1:2 kinetic model, indicative of IFNγ binding to its two receptors (FIG. 14B). IFNγ binds to IFNγR1 with a high affinity ranging from picomolar to nanomolar, and the IFNγ-IFNγR1 complex binds to IFNγR2 with moderate affinity in the nanomolar range (A63)-(A65). Our data show that the affinity of IFNγ to IFNγR1 is 167 nM (2.8 μg/mL), and the IFNγ-IFNγR1 complex affinity to IFNγR2 is 316 nM (5.3 μg/mL). Although our sensor's affinity is on the low end of the affinity for each receptor, this data demonstrates that the receptors maintain activity and responsiveness to IFNγ. The SPR sensograms show that the complex has a higher association rate (kₒₙ) in comparison to the disassociation rate (k_{off}), as expected for the receptor complex (66), with the response returning to baseline at higher time points (>2000 s). These results demonstrate that the individual sensor arms are kinetically active, reversible, and have the two-step binding mechanism.

We determine the selectivity of the sensor by the maximum SPR response of the sensor to injections of common cytokines (FIG. 14C). We recorded kinetic responses to injections of IFNγ, tumor necrosis factor alpha (TNFα), interleukin-18 (IL-18), and phosphate buffered saline (PBS) as a control. We expect no interaction between other cytokines and the receptors, since IFNγ is the only Class II interferon in its family, resulting in receptors with high specificity (A10). For a comparable analyte, we chose IL-18, since it is part of the Class I interferon family (A67). We found that the nanosensors are selective for IFNγ, with no significant difference in signal between the other cytokines from background (PBS). Since nonspecific interactions can interfere with the SPR signal, we recorded IFNγ addition in the absence of sensors as a negative control. We prepared a streptavidin-coated SPR chip without sensors and measured the kinetics for nonspecific adsorption of IFNγ and IL-18 (cytokines with similar size). Results of the negative control studies show no response above background, leading us to conclude that the nonspecific interactions do not interfere with the SPR signal. Given the design of the SPR-based experiment, IFNγR1 will be considerably closer to the surface than IFNγR2 when immobilized. This presents a possibility that the signal generated could be from the binding of IFNγ to IFNγR1 only, not necessarily due to the closing of the hinge and stacking with IFNγR2. To preclude this possibility, we measured the kinetic response from sensors fabricated without IFNγR2. The response for single sensor arms with only IFNγR1 fits a 1:1 binding model with a K_{D} of 93.2 nM (1.6 μg/mL). This result indicates that the response profile of the whole sensor is due to 1:2 binding with IFNγR1 and IFNγR2 and not attributed to only IFNγR1.

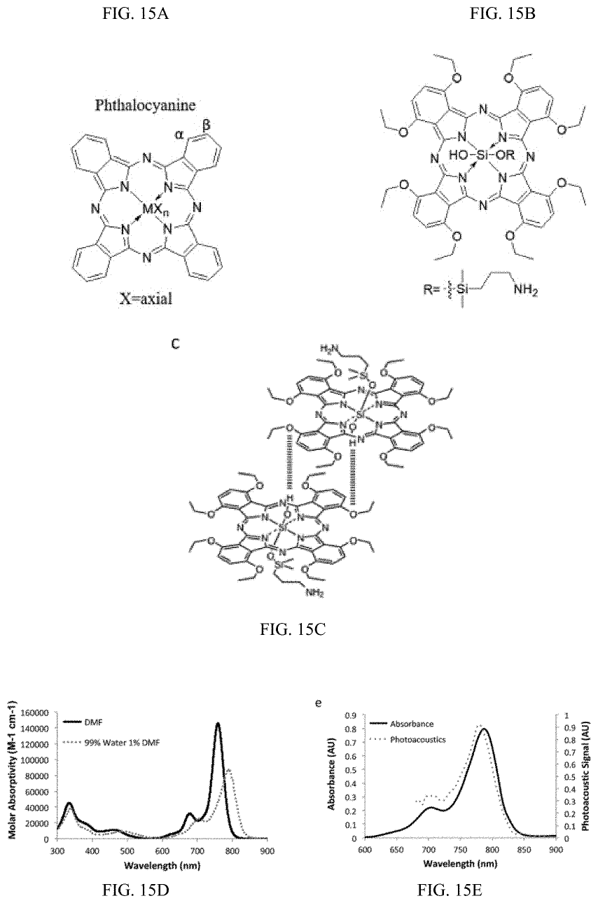

In one embodiment according to the invention, a nanosensor design includes a novel PA reporter based on silicon Pc dyes. Pc dyes are clinically accessible, nontoxic, PA dyes with modifiable physical and optical properties (A68). They are noncytotoxic and produce no phototoxicity since the light necessary for activation during imaging is not sufficient to generate singlet oxygen (A69). FIGS. 15A-15E are diagrams illustrating design of a Pc chromophore, in accordance with an embodiment of the invention, as discussed further below. FIG. 15A shows a Pc chromophore; FIG. 15B shows design of Pc-1; FIG. 15C is a representation of J-aggregate stacking of a Pc-1 dyad demonstrating electron sharing (dotted lines) between π-π orbitals; FIG. 15D is a graph showing Pc-1 absorbance spectra in DMF solvent (solid line) and 99% water 1% DMF (dotted line); and FIG. 15E is a graph showing absorbance and PA spectra of Pc-1-tagged DNA.

In more detail, the optical, physical, or electrical properties of Pc dyes can be modified via addition of a central element (M) or by substitution at the α/β phenyl positions with electron donating/withdrawing groups (FIG. 15A). Pcs organize in supra-molecular stacks, or aggregates, due to strong π-π orbital interactions. Typically, Pcs self-orient into H-aggregates or cofacial stacks, with their two faces parallel and their π orbitals overlapping. Specific molecular architectures can cause the Pcs to stack in a “slipped” configuration, with the edge of one Pc overlapping the centers of its neighboring Pcs, known as J-aggregation. The stacking affects the Pc photophysical properties; the excited state wave functions mix, resulting in two new nondegenerate excited states. Due to selection rules, only transitions to the higher energy excited state are allowed in H-aggregates, while only transitions to the lower energy excited state are allowed in J-aggregates (A47).

Pc-1 is a silicon-centered Pc with ethoxy substituents in each of the eight α-positions (FIG. 15B). We designed Pc-1 to achieve three characteristics, near-infrared (>700 nm, NIR) absorbance, J-aggregate formation, and conjugation to DNA. Pcs substituted with alkoxy groups at all the α-positions generally demonstrate NIR absorbance but show reduced aggregation due to distortion of planarity on the Pc. We selected ethoxy substituents to impart NIR absorbance while minimizing steric repulsion of the alkoxy groups (we attempted to prepare methoxy-substituted Pcs, but the precursors were not sufficiently soluble to complete the synthesis) to maximize any intermolecular aggregation possible with this substitution pattern. The silicon center requires two additional axial substituents. We selected a hydroxyl group and a (3-aminopropyl)dimethylsiloxy group. We chose the hydroxy group to minimize steric repulsion between Pcs, and the amino group to facilitate DNA conjugation. Additionally, the larger aminopropyl substituent hinders cofacial overlap between Pcs. The small hydroxyl group and the bulky aminopropyl group favor the J-aggregate arrangement (one possible arrangement shown in FIG. 15C).

To determine the absorbance properties of the monomer and aggregate, we analyzed Pc-1 in a good solvent system (dimethylformamide, DMF) to inhibit aggregation and a poor solvent system (99% water, 1% DMF) to promote aggregation. The absorbance spectrum of monomeric Pc-1 is consistent with other reported Pc absorbance spectra, a strong absorbance band (the Q-band) with an 1.5×10⁵ M⁻¹ cm⁻¹ absorption coefficient at 759 nm with weaker absorbance bands at 400-500 and 350 nm (FIG. 15D). When Pc-1 disperses from DMF solution into 99% water, its absorbance maximum shifts from 759 to 788 nm, with a decrease in absorbance coefficient of 6.0×10⁴ M⁻¹ cm⁻¹. Both changes in the absorbance spectra are consistent with J-aggregate formation. Shifts in wavelength of less than 25 nm are photoacoustically detectable in in vivo systems (A70). The absorbance maximum of Pc-1 shifts 29 nm (759-788 nm), providing a sufficient difference in wavelength to detect changes in signal. To further characterize the aggregation, we obtained the absorbance spectra of Pc-1 in mixed solvent systems, ranging from pure ethanol to 10% ethanol in water. As the water content in the solvent system increases, the absorbance spectrum decreases in intensity and undergoes a bathochromic shift from 760 to 780 nm. This shift is likely due to a combination of solvatochromism and aggregation, as the bathochromic shift occurs with minimal water addition, but the decrease in molar absorptivity does not occur until the solvent system exceeds 40% water.

Photoactive agents, such as Pc-1, have absorption in the NIR range and produce heat when irradiated with NIR light with nanosecond pulses. This heat generates a thermoelastic expansion of the surrounding environment resulting in acoustic wave propagation. The generation of the PA signal is dependent on the nonradiative excited state decay. Since high fluorescence quantum yields indicate poor PA signal generation, we determined the fluorescence quantum yield of Pc-1 conjugated to DNA to assess the percentage of the radiative excited state decay we can expect from the sensor. The quantum yield (Φ_{f}) of Pc-1-labeled DNA is lower (Φ_{f}=0.0159) than many typical NIR fluorescent dyes (Φ_{f}=0.2-0.3) (71) and silicon Pcs (Φ_{f}=0.1-0.3) (A72). Weak emission indicates that Pc-1 relaxes from its excited state to its ground state through a nonradiative decay process, such as internal conversion, resulting in heat generation (A73-A75).

We note, that face-to-face H-aggregation of the dyes may also generate changes in light absorbance detectable by PA. H- and J-aggregation produce a blue and red shift in spectra, respectively (A76). Our results show the absorbance of Pc-1 undergoes a red-shift in spectrum (increase at 780 nm peak and decrease at 760 nm), suggesting J-aggregation (FIG. 15D) and generating a PA signal at 780 nm (FIG. 15E). However, we did not explicitly determine the dye stacking configurations, and our results may include several aggregate types. In all, by creating Pc-1 with increased absorbance at longer wavelengths, where absorbance of the dye monomers is weak or absent, we obtain a more easily detectable “turn-on” PA signal in the NIR range for incorporation in the nanosensor. It is important to note, that in the context of in vivo imaging, endogenous absorbers, such as oxygenated and deoxygenated hemoglobin, also absorb in the same optical window as NIR dyes (680-900 nm). Compared to contrast agents (i.e., Pc dye), however, the PA signal from hemoglobin is minimal and can be isolated from the target signal with multiwavelength imaging (A77, A78).

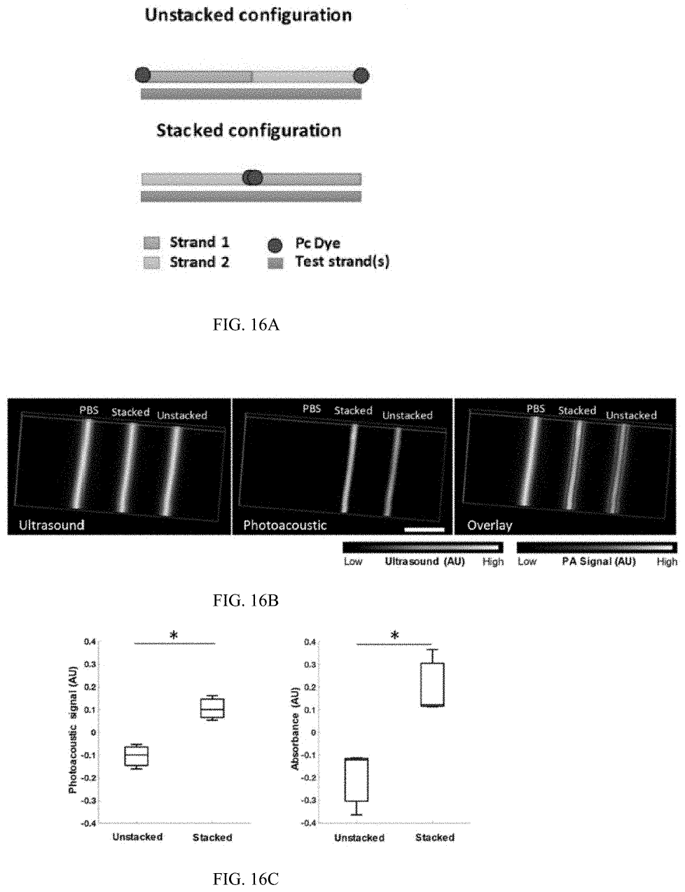

FIGS. 16A-16C are diagrams illustrating photoacoustic responses of test structures in an experiment in accordance with an embodiment of the invention. FIG. 16A is a schematic diagram of test structures; FIG. 16B is a set of 3D images of the test structures' ultrasound, PA response at 780 nm, and overlay; and FIG. 16C is a graph of stacked and unstacked test responses, PA (p=0.0091, n=3, 22.5% increase) and absorbance (p=0.0266, n=3, 47.4% increase). White scale bar=5 mm. Error bars represent the standard deviation of three independent trials. In the experiments of FIGS. 16A-16C, a linear DNA-based structure was used to space the dye molecules in a stacked and an unstacked configuration, in order to find the maximum and minimum expected signals. We designed a pair of DNA and Pc-1 sequences in two configurations based on previous studies that show that these structures force Pc dyes to stack or unstack based on the distance (54). These model structures represent the signal produced from ideal stacking (and unstacking) conditions and reflect the maximum and minimum signal. Our nanosensor configuration cannot replicate the exact geometry of the model system and thus would not match those limits. Variability in our nanosensor's geometry may interfere with stacking, resulting in suboptimal alignment. Signal intensity of dye molecules varies greatly upon proximity. Dye pairs are expected to exhibit a stronger absorbance and PA signal when in the stacked configuration because of resonant interactions when in close proximity. This design contains two dye-linked sequences with interaction distances of 17 nm to produce an unstacked configuration and <1 nm to promote a stacked configuration (FIG. 16A). Anchoring two-dye molecules 17 nm apart prevents the overlap of dye pair sites available for J-stacking, thus generating a weaker PA signal than those attached in close proximity. The utility of aggregation in the PA sensor signal stems from the resonant cross-section of the engineered dye being sensitive to dye pair separation.

Resonant profiles of dye aggregation yield an increase in both absorbance and PA signal, since PA output depends on optical absorption. Our dye design achieves this by tuning the resonant profile to decrease cross-sectional scattering, resulting in an increase in absorption. PA images are inherent 3-dimensional measurements that take advantage of the existing ultrasound imaging technique to provide high-resolution imaging without the need for a new detector. We imaged capillary tubes filled with a solution containing these two DNA sequences and PBS as a control in 3D (FIG. 16B). The sequences were imaged in three independent trials and normalized to the mean signal. The images show 3D renderings of the ultrasound and PA response from each tube, PBS only, stacked configuration, and unstacked configuration. While there was no difference in ultrasound signatures of the tubes, dyes anchored on DNA at different distances from one another (unstacked ˜17 nm and stacked ˜1 nm) generate distinguishable PA signals compared to PBS, as noted on the image. Pc dyes increase the PA signal when stacked. Thus, we expect a higher signal from the tube containing the stacked configuration in comparison to the unstacked. Quantitative analysis of the PA B-scan images (FIG. 16C) shows that the stacked configuration exhibits a 22.5% higher response in comparison to the unstacked, as expected. The significant difference (p=0.0221) between stacked and unstacked tests indicates that two Pc dyes in close proximity lead to an increase in the PA signal.

Binding parameters between a dye pair, such as proximity and stacking of PA dyes linked to the DNA, can be monitored by the change in the PA signal. In the open state, the hinge arms are open, resulting in PA dye pair separation, decreasing the probability of dye stacking events. In the closed state, the distance between dyes is decreased, leading to dye stacking and a change in absorbance. To create a functional PA sensor for IFNγ, we incorporated Pc dyes 4 nm from the ssDNA hinge of each arm in the complex (FIGS. 13A and 13B). The resulting PA signal is low when the nanosensor is open and high when it is closed. To determine the site of each Pc dye, we evaluated the peak PA signal of three different dye sites on the DNA scaffold based on the geometry of the nanosensor. We down-selected the site with the largest change in the PA signal for the final sensor (data not shown).

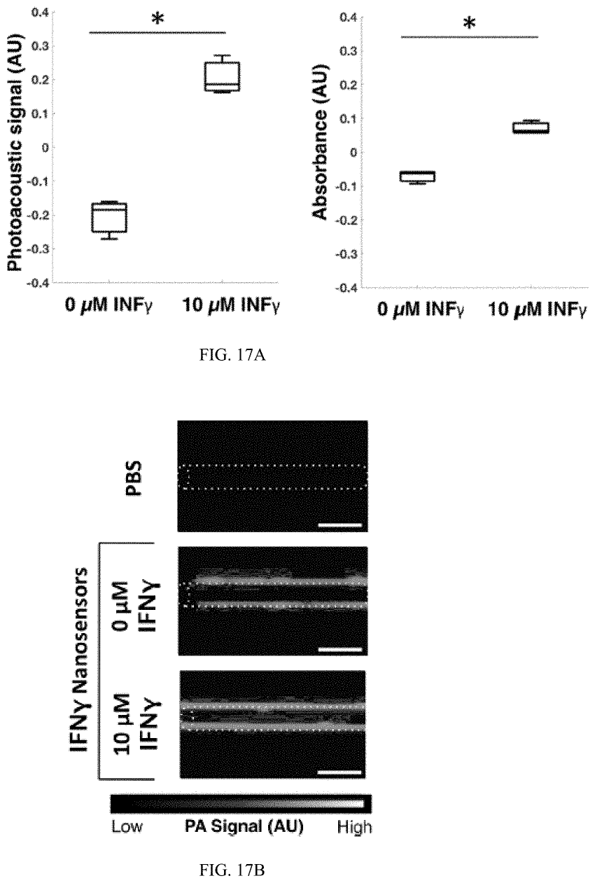

After characterizing the different components of the nanosensor, the overall nanosensor architecture was utilized in a proof-of-concept measurement of IFNγ based on the nanosensor's PA response, for which results are shown in FIGS. 17A and 17B. FIG. 17A is set of graphs showing response of the IFNγ nanosensor (780 nm) to buffer and 10 μM IFNγ (169 μg/mL) in buffer, PA signal (p=0.0009, n=3, 55.3% increase) and absorbance (p=0.0010, n=3, 37.7% increase). FIG. 17B is a set of images of 2D scans of the nanosensor's PA response in capillary tubes (dotted lines). White scale bar=1 mm. Error bars represent the standard deviation of three independent trials.

In more detail, in the experiments for FIGS. 17A and 17B, we imaged 1 μM nanosensor solutions in PBS with or without IFNγ in three independent trials. Here, the 0 μM solution ensures that the nanosensor is in the open conformation, producing minimal PA signal. To promote the closed nanosensor conformation and induce maximal change in the PA signal, the 10 μM IFNγ solution (169 μg/mL) was used with 2.5-fold excess of the binding sites available on the nanosensor. We injected the nanosensor solutions containing 0 or 10 μM IFNγ into polyethelyene tubing and secured them in a PA phantom box for imaging. We acquired 2D PA scans and averaged three regions of interest to generate a spectrum for each sample. We replicated this experiment two additional times with different nanosensor batches for each trial. FIG. 17B shows the change in absorbance and PA signal between the three trials at the peak wavelength for dye stacking (780 nm). Of the two conformations, our data show that the closed structure (10 μM IFNγ) has a 55.3% higher PA peak at 780 nm (p=0.0009, n=3) and a 37.7% increase in absorbance (p=0.0010, n=3). This increase in both absorbance and PA signal is also seen during dye stacking in the test structures (FIGS. 16A-16C). The nanosensor's in vitro conformational response leads to the conclusion whereby Pc dyes move into close proximity and stack, consequently increasing their absorbance and PA signal. Concomitant increases in sensitivity and resolution provided by both increased absorbance and PA signal increases coalesce as an optimal mechanism for a PA signal platform.

Conclusions: IFNγ Sensor

The above experiments of FIGS. 13A-17B illustrate proof-of-concept of a PA nanosensor with (a) a reversible recognition element driven by receptor binding, (b) a stackable Pc dye for a turn-on PA signal, and (c) a DNA-based nanostructure scaffold to facilitate an increase in PA response upon IFNγ binding. Each sensor arm binds selectively and reversibly to IFNγ with affinities of 167 (2.8 μg/mL) and 316 nM (5.3 μg/mL). A Pc-based dye was synthesized, Pc-1, and increased its PA signal by 22.5% when stacked or aggregated in solution. Finally, nanosensors composed of receptors and Pc-1 bound to a DNA scaffold demonstrated a change in PA signal upon IFNγ binding. Together, these results demonstrate a photoacoustically active nanosensor for the detection of the protein IFNγ.

This proof-of-concept sensor architecture was designed for free solution characterization, but with the end goal of in vivo detection in mind. Notably, the application of this PA-based sensing technology is not targeted toward ex vivo or in situ analysis, as current fluorescence-based techniques have better sensitivity compared to PA-based. Nevertheless, the mechanism of the sensor should be translatable to future designs using more sensitive optical techniques. This sensor can be developed further for in vivo use by improving the sensitivity and robustness. In its configuration of FIGS. 13A-17B, the dynamic range indicated by SPR studies is greater than the relevant physiological range of IFNγ in the bloodstream (0.5-100 pM or 0.01-2 ng/mL) (A79), (A80). The sensitivity can be optimized by increasing the number of Pc dyes, improving the sensitivity of the instrument, or modifying the placement of the dye and receptor sites. A second consideration for in vivo use is the robustness of the nanosensor in the complex environment of the bloodstream. In particular, the DNA backbone of the sensor is susceptible to enzymatic degradation in the physiological environment with lifetimes of up to a few days (A58), (A81). For longer time scales, the DNA structure may be strengthened by cross-linking (A82) or employing shielding mechanisms, such as polyethylene glycol coatings (A83), to delay degradation (A84). Another strategy is to tether the sensor arms onto a scaffold (i.e., stent) that can be replaced to replenish the sensors once degradation occurs. This strategy can also potentially improve the characteristics of the sensor. The stent provides a large surface area on which sensor arms can be concentrated for increased PA output, thereby providing more sensitive in vivo imaging. Tethering to a solid support can also reduce the probability of cross-linking between different sensor arms, thus reducing background signal.

Definitions and Discussion of Terminology

Although various specific cytokines have been discussed herein, it will be appreciated that photoacoustic techniques of other types of cytokines, including multiplexed groups of cytokines, can be performed.

In addition, although application to monitoring of, for example, the cytokine storms involved with COVID-19 have been discussed, it will be appreciated that cytokines can be monitored for other conditions, including systemic cytokine storm syndromes (such as, for example, CAR-T immunotoxicity, HLH, sepsis, and others) and localized cytokine signaling activity (such as, for example, rheumatoid arthritis and others.

As used herein, it will be appreciated that a “nucleic acid-based nanosensor” need not be composed exclusively of one or more nucleic acids, but rather includes at least a substantial portion of its structure being made of one or more nucleic acids. Likewise, a “DNA origami based nanosensor” need not be composed exlusively of DNA origami, but rather includes at least a substantial portion of its structure being made of DNA origami.

As used herein, an “indocyanine green analog,” a “phthalocyanine chromophore,” and a “BODIPY analog” are not limited to the particular instances discussed, but can include other such analogs and chromophores.

As used herein, a molecular scaffold can, for example, be or include a polycyclic molecular scaffold, and in one example can be a xanthene molecular scaffold.

As used herein, an “injectable agent” can, for example, include one or more components that render injected substances suitable for injection in vivo in an animal or human subject, and can further include, for example, one or more components that encourage components of an injected fluid to remain in an injected space or tissue within the animal or human subject.

As used herein, “nucleic acid” refers to a macromolecule composed of chains (a polymer or an oligomer) of monomeric nucleotide. The most common nucleic acids are deoxyribonucleic acid (DNA) and ribonucleic acid (RNA). It should be further understood that nucleic acids can include artificial nucleic acids such as peptide nucleic acid (PNA), morpholino, locked nucleic acid (LNA), glycol nucleic acid (GNA) and threose nucleic acid (TNA), among others. In various embodiments of the present invention, nucleic acids can be derived from a variety of sources such as bacteria, virus, humans, and animals, as well as sources such as plants and fungi, among others. The source can be a pathogen. Alternatively, the source can be a synthetic organism. Nucleic acids can, for example, be genomic, extrachromosomal or synthetic. In addition, the term “nucleic acid,” is used herein to include a polymeric form of nucleotides of any length, including, but not limited to, ribonucleotides or deoxyribonucleotides. Further, the term refers only to the primary structure of the molecule. Thus, in certain embodiments the term can include triple-, double- and single-stranded DNA, PNA, complementary DNA (cDNA), as well as triple-, double- and single-stranded RNA. It can also include modifications, such as by methylation and/or by capping, and unmodified forms of a polynucleotide. More particularly, the term “nucleic acid,” includes polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, polyamide (e.g., peptide nucleic acids (PNAs)) and polymorpholino (commercially available from Anti-Virals, Inc., Corvallis, Oreg., U.S.A., as Neugene) polymers, and other synthetic sequence-specific nucleic acid polymers providing that the polymers contain nucleobases in a configuration which allows for base pairing and base stacking, such as is found in DNA and RNA. In addition, a “nucleic acid” can include a plasmid DNA (pDNA), such as a plasmid DNA vector.

As used herein, a “modification of a nucleic acid sequence” refers to a mutant nucleic acid (e.g., DNA) that is not naturally occurring, and that has a mutation relative to a reference nucleic acid, that is, by an alteration of the nucleotide sequence of the reference nucleic acid sequence, such as by substitution, insertion or deletion of one or more nucleotides. In some embodiments, the mutation can be a missense mutation, which is a type of nonsynonymous substitution that is a point mutation in which a single nucleotide change results in a codon that codes for a different amino acid.

In some embodiments, a nucleic acid molecule comprising a modification of a nucleic acid sequence can be isolated or recombinant. In addition, such a modification of a nucleic acid sequence can be produced using techniques of cell-free protein synthesis, which produce protein using biological machinery in a cell-free system, without the use of living cells. Cell free expression systems can, for example, be used, that use linear DNA sequences propagated by polymerase chain reaction (PCR) reactions.

### REFERENCES

1. Moore, B. J. B.; June, C. H., Cytokine release syndrome in severe COVID-19. Science 2020, eabb8925. 32303591

2. Huang, K. J.; Su, I. J.; Theron, M.; Wu, Y. C.; Lai, S. K.; Liu, C. C.; Lei, H. Y., An interferon-γ-related cytokine storm in SARS patients. Journal of Medical Virology 2005, 75 (2), 185-194. PMC7166886

3. Morichi, S.; Kawashima, H.; Ioi, H.; Yamanaka, G.; Kashiwagi, Y.; Hoshika, A., High production of interleukin-10 and interferon-γ in influenza-associated MERS in the early phase. Pediatrics International 2012, 54 (4), 536-538. 22830540

4. Liu, J.; Li, S.; Liu, J.; Liang, B.; Wang, X.; Wang, H.; Li, W.; Tong, Q.; Yi, J.; Zhao, L.; Xiong, L.; Guo, C.; Tian, J.; Luo, J.; Yao, J.; Pang, R.; Shen, H.; Peng, C.; Liu, T.; Zhang, Q.; Wu, J.; Xu, L.; Lu, S.; Wang, B.; Weng, Z.; Han, C.; Zhu, H.; Zhou, R.; Zhou, H.; Chen, X.; Ye, P.; Zhu, B.; Wang, L.; Zhou, W.; He, S.; He, Y.; Jie, S.; Wei, P.; Zhang, J.; Lu, Y.; Wang, W.; Zhang, L.; Li, L.; Zhou, F.; Wang, J.; Dittmer, U.; Lu, M.; Hu, Y.; Yang, D.; Zheng, X., Longitudinal characteristics of lymphocyte responses and cytokine profiles in the peripheral blood of SARS-CoV-2 infected patients. EBioMedicine 2020, 102763. PMC7195532

5. Ragab, D.; Salah Eldin, H.; Taeimah, M.; Khattab, R.; Salem, R., The COVID-19 Cytokine Storm; What We Know So Far. Frontiers in Immunology 2020, 11 (1446). PMC7308649

6. Riegler, L. L.; Jones, G. P.; Lee, D. W., Current approaches in the grading and management of cytokine release syndrome after chimeric antigen receptor T-cell therapy. Ther Clin Risk Manag 2019, 15, 323-335. PMC6400118

7. Morales, J. M.; Pawle, R.; Akkilic, N.; Luo, Y.; Xavierselvan, M.; Albokhari, R.; Calderon, I. A. C.; Selfridge, S.; Minns, R.; Takiff, L.; Clark, H., DNA-based photoacoustic nanosensor for interferon gamma detection. ACS Sensors 2019, 4 (5), 1313-1322. 30973005

8. Wang, P.; Meyer, T. A.; Pan, V.; Dutta, P. K.; Ke, Y., The beauty and utility of DNA origami. Chem 2017, 2 (3), 359-382.

9. Wang, L. V.; Hu, S., Photoacoustic tomography: in vivo imaging from organelles to organs. Science 2012, 335 (6075), 1458-1462. PMC3322413

10. Zou, C.; Wu, B.; Dong, Y.; Song, Z.; Zhao, Y.; Ni, X.; Yang, Y.; Liu, Z., Biomedical photoacoustics: fundamentals, instrumentation and perspectives on nanomedicine. Int J Nanomedicine 2016, 12, 179-195. PMC5191855

11. Bhimra, A.; Morgan, R. L.; Shumaker, A. H.; Lavergne, V.; Baden, L.; Cheng, V. C.-C.; Edwards, K. M.; Gandhi, R.; Gallagher, J.; Muller, W. J.; O'Horo, J. C.; Shoham, S.; Murad, M. H.; Mustafa, R. A.; Sultan, S.; Falck-Ytter, Y. Infectious Diseases Society of America Guidelines on the Treatment and Management of Patients with COVID-19; Infectious Diseases Society of America (IDSA): idsociety.org, 2020.

12. Critical Care—Cytokine Storm Syndrome. Brigham and Women's Hospital:

https://covidprotocols.org/protocols/critical-care/#cytokine-storm-syndrome.

13. Lee, D. W.; Gardner, R.; Porter, D. L.; Louis, C. U.; Ahmed, N.; Jensen, M.; Grupp, S. A.; Mackall, C. L., Current concepts in the diagnosis and management of cytokine release syndrome. Blood 2014, 124(2), 188-195. PMC4093680

14. Clay, T. M.; Hobeika, A. C.; Mosca, P. J.; Lyerly, H. K.; Morse, M. A., Assays for monitoring cellular immune responses to active immunotherapy of cancer. Clinical Cancer Research 2001, 7 (5), 1127-1135. 11350875

15. Aziz, N., Measurement of Circulating Cytokines and Immune-Activation Markers by Multiplex Technology in the Clinical Setting: What Are We Really Measuring? For Immunopathol Dis Therap 2015, 6 (1-2), 19-22.

16. Calabrese, C.; Rajendram, P.; Sacha, G.; Calabrese, L., Practical aspects of targeting IL-6 in COVID-19 disease. Cleveland Clinic Journal of Medicine 2020, Online ahead of print. 32409439

17. Knight, V.; Long, T.; Meng, Q. H.; Linden, M. A.; Rhoads, D. D., Variability in the Laboratory Measurement of Cytokines: A Longitudinal Summary of a College of American Pathologists Proficiency Testing Survey. Archives of Pathology & Laboratory Medicine 2020, 144 (10), 1230-1233. 32401053

18. Davila, M. L.; Riviere, I.; Wang, X.; Bartido, S.; Park, J.; Curran, K.; Chung, S. S.; Stefanski, J.; Borquez-Ojeda, O.; Olszewska, M.; Qu, J.; Wasielewska, T.; He, Q.; Fink, M.; Shinglot, H.; Youssif, M.; Satter, M.; Wang, Y.; Hosey, J.; Quintanilla, H.; Halton, E.; Bernal, Y.; Bouhassira, D. C.; Arcila, M. E.; Gonen, M.; Roboz, G. J.; Maslak, P.; Douer, D.; Frattini, M. G.; Giralt, S.; Sadelain, M.; Brentjens, R., Efficacy and toxicity management of 19-28z CAR T cell therapy in B cell acute lymphoblastic leukemia. Science translational medicine 2014, 6 (224), 224ra25. PMC4684949

19. Russell, S. M.; Alba-Patiño, A.; Barón, E.; Borges, M.; Gonzalez-Freire, M.; de la Rica, R., Biosensors for Managing the COVID-19 Cytokine Storm: Challenges Ahead. ACS Sensors 2020. PMC7299396

20. Del Valle, D. M.; Kim-Schulze, S.; Huang, H.-H.; Beckmann, N. D.; Nirenberg, S.; Wang, B.; Lavin, Y.; Swartz, T. H.; Madduri, D.; Stock, A.; Marron, T. U.; Xie, H.; Patel, M.; Tuballes, K.; Van Oekelen, O.; Rahman, A.; Kovatch, P.; Aberg, J. A.; Schadt, E.; Jagannath, S.; Mazumdar, M.; Charney, A. W.; Firpo-Betancourt, A.; Mendu, D. R.; Jhang, J.; Reich, D.; Sigel, K.; Cordon-Cardo, C.; Feldmann, M.; Parekh, S.; Merad, M.; Gnjatic, S., An inflammatory cytokine signature predicts COVID-19 severity and survival. Nature Medicine 2020, 26 (10), 1636-1643. 32839624

21. De Jager, W.; Bourcier, K.; Rijkers, G. T.; Prakken, B. J.; Seyfert-Margolis, V., Prerequisites for cytokine measurements in clinical trials with multiplex immunoassays. BMC Immunol 2009, 10, 52-52. PMC2761376

22. Goldman, J. D.; Lye, D. C. B.; Hui, D. S.; Marks, K. M.; Bruno, R.; Montejano, R.; Spinner, C. D.; Galli, M.; Ahn, M. Y.; Nahass, R. G.; Chen, Y. S.; SenGupta, D.; Hyland, R. H.; Osinusi, A. O.; Cao, H.; Blair, C.; Wei, X.; Gaggar, A.; Brainard, D. M.; Towner, W. J.; Muñoz, J.; Mullane, K. M.; Marty, F. M.; Tashima, K. T.; Diaz, G.; Subramanian, A., Remdesivir for 5 or 10 Days in Patients with Severe Covid-19. The New England journal of medicine 2020. PMC7377062

23. Jamilloux, Y.; Henry, T.; Belot, A.; Viel, S.; Fauter, M.; El Jammal, T.; Walzer, T.; François, B.; Sève, P., Should we stimulate or suppress immune responses in COVID-19? Cytokine and anti-cytokine interventions. Autoimmun Rev 2020, 19 (7), 102567-102567. PMC7196557

24. Guzik, T. J.; Mohiddin, S. A.; Dimarco, A.; Patel, V.; Savvatis, K.; Marelli-Berg, F. M.; Madhur, M. S.; Tomaszewski, M.; Maffia, P.; D'Acquisto, F.; Nicklin, S. A.; Marian, A. J.; Nosalski, R.; Murray, E. C.; Guzik, B.; Berry, C.; Touyz, R. M.; Kreutz, R.; Wang, D. W.; Bhella, D.; Sagliocco, O.; Crea, F.; Thomson, E. C.; McInnes, I. B., COVID-19 and the cardiovascular system: implications for risk assessment, diagnosis, and treatment options. Cardiovascular Research 2020, 116 (10), 1666-1687. PMC7197627

25. Shang, J.; Ye, G.; Shi, K.; Wan, Y.; Luo, C.; Aihara, H.; Geng, Q.; Auerbach, A.; Li, F., Structural basis of receptor recognition by SARS-CoV-2. Nature 2020, 581 (7807), 221-224. PMC7328981

26. Bosch, B. J.; van der Zee, R.; de Haan, C. A. M.; Rottier, P. J. M., The Coronavirus Spike Protein Is a Class I Virus Fusion Protein: Structural and Functional Characterization of the Fusion Core Complex. Journal of Virology 2003, 77 (16), 8801-8811. PMC167208

27. Huang, C.; Wang, Y.; Li, X.; Ren, L.; Zhao, J.; Hu, Y.; Zhang, L.; Fan, G.; Xu, J.; Gu, X.; Cheng, Z.; Yu, T.; Xia, J.; Wei, Y.; Wu, W.; Xie, X.; Yin, W.; Li, H.; Liu, M.; Xiao, Y.; Gao, H.; Guo, L.; Xie, J.; Wang, G.; Jiang, R.; Gao, Z.; Jin, Q.; Wang, J.; Cao, B., Clinical features of patients infected with 2019 novel coronavirus in Wuhan, China. The Lancet 2020, 395 (10223), 497-506. PMC7265838

28. Chen, G.; Wu, D.; Guo, W.; Cao, Y.; Huang, D.; Wang, H.; Wang, T.; Zhang, X.; Chen, H.; Yu, H.; Zhang, X.; Zhang, M.; Wu, S.; Song, J.; Chen, T.; Han, M.; Li, S.; Luo, X.; Zhao, J.; Ning, Q., Clinical and immunological features of severe and moderate coronavirus disease 2019. The Journal of Clinical Investigation 2020, 130 (5). PMC7190990

29. Lucas, C.; Wong, P.; Klein, J.; Castro, T. B. R.; Silva, J.; Sundaram, M.; Ellingson, M. K.; Mao, T.; Oh, J. E.; Israelow, B.; Takahashi, T.; Tokuyama, M.; Lu, P.; Venkataraman, A.; Park, A.; Mohanty, S.; Wang, H.; Wyllie, A. L.; Vogels, C. B. F.; Earnest, R.; Lapidus, S.; Ott, I. M.; Moore, A. J.; Muenker, M. C.; Fournier, J. B.; Campbell, M.; Odio, C. D.; Casanovas-Massana, A.; Obaid, A.; Lu-Culligan, A.; Nelson, A.; Brito, A.; Nunez, A.; Martin, A.; Watkins, A.; Geng, B.; Kalinich, C.; Harden, C.; Todeasa, C.; Jensen, C.; Kim, D.; McDonald, D.; Shepard, D.; Courchaine, E.; White, E. B.; Song, E.; Silva, E.; Kudo, E.; DeIuliis, G.; Rahming, H.; Park, H.-J.; Matos, I.; Nouws, J.; Valdez, J.; Fauver, J.; Lim, J.; Rose, K.-A.; Anastasio, K.; Brower, K.; Glick, L.; Sharma, L.; Sewanan, L.; Knaggs, L.; Minasyan, M.; Batsu, M.; Petrone, M.; Kuang, M.; Nakahata, M.; Campbell, M.; Linehan, M.; Askenase, M. H.; Simonov, M.; Smolgovsky, M.; Sonnert, N.; Naushad, N.; Vijayakumar, P.; Martinello, R.; Datta, R.; Handoko, R.; Bermejo, S.; Prophet, S.; Bickerton, S.; Velazquez, S.; Alpert, T.; Rice, T.; Khoury-Hanold, W.; Peng, X.; Yang, Y.; Cao, Y.; Strong, Y.; Herbst, R.; Shaw, A. C.; Medzhitov, R.; Schulz, W. L.; Grubaugh, N. D.; Dela Cruz, C.; Farhadian, S.; Ko, A. I.; Omer, S. B.; Iwasaki, A.; Yale, I. T., Longitudinal analyses reveal immunological misfiring in severe COVID-19. Nature 2020, 584 (7821), 463-469. PMC7477538

30. DeDiego, M. L.; Nieto-Torres, J. L.; Regla-Nava, J. A.; Jimenez-Guardeño, J. M.; Fernandez-Delgado, R.; Fett, C.; Castaño-Rodriguez, C.; Perlman, S.; Enjuanes, L., Inhibition of NF-κB-mediated inflammation in severe acute respiratory syndrome coronavirus-infected mice increases survival. Journal of Virology 2014, 88 (2), 913-924. PMC3911641

31. Catanzaro, M.; Fagiani, F.; Racchi, M.; Corsini, E.; Govoni, S.; Lanni, C., Immune response in COVID-19: addressing a pharmacological challenge by targeting pathways triggered by SARS-CoV-2. Signal Transduct Target Ther 2020, 5 (1), 84-84. PMC7255975

32. Hadjadj, J.; Yatim, N.; Barnabei, L.; Corneau, A.; Boussier, J.; Smith, N.; Péré, H.; Charbit, B.; Bondet, V.; Chenevier-Gobeaux, C.; Breillat, P.; Carlier, N.; Gauzit, R.; Morbieu, C.; Pène, F.; Marin, N.; Roche, N.; Szwebel, T.-A.; Merkling, S. H.; Treluyer, J.-M.; Veyer, D.; Mouthon, L.; Blanc, C.; Tharaux, P.-L.; Rozenberg, F.; Fischer, A.; Duffy, D.; Rieux-Laucat, F.; Kernéis, S.; Terrier, B., Impaired type I interferon activity and inflammatory responses in severe COVID-19 patients. Science 2020, 369 (6504), 718-724. PMC7402632

33. Acharya, D.; Liu, G.; Gack, M. U., Dysregulation of type I interferon responses in COVID-19. Nature Reviews Immunology 2020, 20 (7), 397-398. PMC7249038

34. Lee, J. S.; Shin, E.-C., The type I interferon response in COVID-19: implications for treatment. Nature Reviews Immunology 2020, 20 (10), 585-586. 32788708

35. Wang, D.; Hu, B.; Hu, C.; Zhu, F.; Liu, X.; Zhang, J.; Wang, B.; Xiang, H.; Cheng, Z.; Xiong, Y.; Zhao, Y.; Li, Y.; Wang, X.; Peng, Z., Clinical Characteristics of 138 Hospitalized Patients With 2019 Novel Coronavirus-Infected Pneumonia in Wuhan, China. JAMA 2020, 323 (11), 1061-1069. PMC7042881

36. Diao, B.; Wang, C.; Tan, Y.; Chen, X.; Liu, Y.; Ning, L.; Chen, L.; Li, M.; Liu, Y.; Wang, G.; Yuan, Z.; Feng, Z.; Zhang, Y.; Wu, Y.; Chen, Y., Reduction and Functional Exhaustion of T Cells in Patients With Coronavirus Disease 2019 (COVID-19). 2020, 11 (827). PMC7205903

37. Zheng, H.-Y.; Zhang, M.; Yang, C.-X.; Zhang, N.; Wang, X.-C.; Yang, X.-P.; Dong, X.-Q.; Zheng, Y.-T., Elevated exhaustion levels and reduced functional diversity of T cells in peripheral blood may predict severe progression in COVID-19 patients. Cellular & Molecular Immunology 2020, 17 (5), 541-543. PMC7091621

38. Mathew, D.; Giles, J. R.; Baxter, A. E.; Oldridge, D. A.; Greenplate, A. R.; Wu, J. E.; Alanio, C.; Kuri-Cervantes, L.; Pampena, M. B.; D'Andrea, K.; Manne, S.; Chen, Z.; Huang, Y. J.; Reilly, J. P.; Weisman, A. R.; Ittner, C. A. G.; Kuthuru, O.; Dougherty, J.; Nzingha, K.; Han, N.; Kim, J.; Pattekar, A.; Goodwin, E. C.; Anderson, E. M.; Weirick, M. E.; Gouma, S.; Arevalo, C. P.; Bolton, M. J.; Chen, F.; Lacey, S. F.; Ramage, H.; Cherry, S.; Hensley, S. E.; Apostolidis, S. A.; Huang, A. C.; Vella, L. A.; Betts, M. R.; Meyer, N. J.; Wherry, E. J., Deep immune profiling of COVID-19 patients reveals distinct immunotypes with therapeutic implications. Science 2020, 369 (6508), eabc8511. PMC7402624

39. Zhou, F.; Yu, T.; Du, R.; Fan, G.; Liu, Y.; Liu, Z.; Xiang, J.; Wang, Y.; Song, B.; Gu, X.; Guan, L.; Wei, Y.; Li, H.; Wu, X.; Xu, J.; Tu, S.; Zhang, Y.; Chen, H.; Cao, B., Clinical course and risk factors for mortality of adult inpatients with COVID-19 in Wuhan, China: a retrospective cohort study. Lancet 2020, 395 (10229), 1054-1062. PMC7270627

40. Mudd, P. A.; Crawford, J. C.; Turner, J. S.; Souquette, A.; Reynolds, D.; Bender, D.; Bosanquet, J. P.; Anand, N. J.; Striker, D. A.; Martin, R. S.; Boon, A. C. M.; House, S. L.; Remy, K. E.; Hotchkiss, R. S.; Presti, R. M.; OHalloran, J. A.; Powderly, W. G.; Thomas, P. G.; Ellebedy, A. H., Targeted Immunosuppression Distinguishes COVID-19 from Influenza in Moderate and Severe Disease. medRxiv 2020, 2020.05.28.20115667. PMC7274224

41. Yang, L.; Liu, S.; Liu, J.; Zhang, Z.; Wan, X.; Huang, B.; Chen, Y.; Zhang, Y., COVID-19: immunopathogenesis and Immunotherapeutics. Signal Transduct Target Ther 2020, 5 (1), 128.

42. Laing, A. G.; Lorenc, A.; del Molino del Barrio, I.; Das, A.; Fish, M.; Monin, L.; Muñoz-Ruiz, M.; McKenzie, D. R.; Hayday, T. S.; Francos-Quijorna, I.; Kamdar, S.; Joseph, M.; Davies, D.; Davis, R.; Jennings, A.; Zlatareva, I.; Vantourout, P.; Wu, Y.; Sofra, V.; Cano, F.; Greco, M.; Theodoridis, E.; Freedman, J.; Gee, S.; Chan, J. N. E.; Ryan, S.; Bugallo-Blanco, E.; Peterson, P.; Kisand, K.; Haljasmägi, L.; Chadli, L.; Moingeon, P.; Martinez, L.; Merrick, B.; Bisnauthsing, K.; Brooks, K.; Ibrahim, M. A. A.; Mason, J.; Lopez Gomez, F.; Babalola, K.; Abdul-Jawad, S.; Cason, J.; Mant, C.; Seow, J.; Graham, C.; Doores, K. J.; Di Rosa, F.; Edgeworth, J.; Shankar-Hari, M.; Hayday, A. C., A dynamic COVID-19 immune signature includes associations with poor prognosis. Nature Medicine 2020, Online ahead of print. 32807934

43. Merad, M.; Martin, J. C., Pathological inflammation in patients with COVID-19: a key role for monocytes and macrophages. Nature Reviews Immunology 2020, 20 (6), 355-362. PMC7201395

44. Skevaki, C.; Fragkou, P. C.; Cheng, C.; Xie, M.; Renz, H., Laboratory characteristics of patients infected with the novel SARS-CoV-2 virus. Journal of Infection 2020, 81 (2), 205-212. PMC7306198

45. Herold, T.; Jurinovic, V.; Arnreich, C.; Lipworth, B. J.; Hellmuth, J. C.; von Bergwelt-Baildon, M.; Klein, M.; Weinberger, T., Elevated levels of IL-6 and CRP predict the need for mechanical ventilation in COVID-19. J Allergy Clin Immunol 2020, 146 (1), 128-136.e4. PMC7233239

46. Wang, Y.; Zhang, D.; Du, G.; Du, R.; Zhao, J.; Jin, Y.; Fu, S.; Gao, L.; Cheng, Z.; Lu, Q.; Hu, Y.; Luo, G.; Wang, K.; Lu, Y.; Li, H.; Wang, S.; Ruan, S.; Yang, C.; Mei, C.; Wang, Y.; Ding, D.; Wu, F.; Tang, X.; Ye, X.; Ye, Y.; Liu, B.; Yang, J.; Yin, W.; Wang, A.; Fan, G.; Zhou, F.; Liu, Z.; Gu, X.; Xu, J.; Shang, L.; Zhang, Y.; Cao, L.; Guo, T.; Wan, Y.; Qin, H.; Jiang, Y.; Jaki, T.; Hayden, F. G.; Horby, P. W.; Cao, B.; Wang, C., Remdesivir in adults with severe COVID-19: a randomised, doubleblind, placebo-controlled, multicentre trial. Lancet 2020, 395 (10236), 1569-1578. PMC7190303

47. Davoudi-Monfared, E.; Rahmani, H.; Khalili, H.; Hajiabdolbaghi, M.; Salehi, M.; Abbasian, L.; Kazemzadeh, H.; Yekaninejad, M. S., A Randomized Clinical Trial of the Efficacy and Safety of Interferon β-1a in Treatment of Severe COVID-19. Antimicrobial agents and chemotherapy 2020, 64 (9). PMC7449227

48. Zhou, Q.; Chen, V.; Shannon, C. P.; Wei, X.-S.; Xiang, X.; Wang, X.; Wang, Z.-H.; Tebbutt, S. J.; Kollmann, T. R.; Fish, E. N., Interferon-α2b Treatment for COVID-19. Frontiers in Immunology 2020, 11 (1061). 32574262

49. Khan, N. A., Anakinra for severe forms of COVID-19. The Lancet Rheumatology 2020, 2 (10), e586-e587.

50. Michot, J.-M.; Albiges, L.; Chaput, N.; Saada, V.; Pommeret, F.; Griscelli, F.; Balleyguier, C.; Besse, B.; Marabelle, A.; Netzer, F.; Merad, M.; Robert, C.; Barlesi, F.; Gachot, B.; Stoclin, A., Tocilizumab, an anti-IL6 receptor antibody, to treat Covid-19-related respiratory failure: a case report. Annals of Oncology: Official Journal of the European Society for Medical Oncology 2020, Epub ahead of print. PMC7136869

51. Biran, N.; Ip, A.; Ahn, J.; Go, R. C.; Wang, S.; Mathura, S.; Sinclaire, B. A.; Bednarz, U.; Marafelias, M.; Hansen, E.; Siegel, D. S.; Goy, A. H.; Pecora, A. L.; Sawczuk, I. S.; Koniaris, L. S.; Simwenyi, M.; Varga, D. W.; Tank, L. K.; Stein, A. A.; Allusson, V.; Lin, G. S.; Oser, W. F.; Tuma, R. A.; Reichman, J.; Brusco, L., Jr.; Carpenter, K. L.; Costanzo, E. J.; Vivona, V.; Goldberg, S. L., Tocilizumab among patients with COVID-19 in the intensive care unit: a multicentre observational study. The Lancet Rheumatology 2020. PMC7428303

52. Wiersinga, W. J.; Rhodes, A.; Cheng, A. C.; Peacock, S. J.; Prescott, H. C., Pathophysiology, Transmission, Diagnosis, and Treatment of Coronavirus Disease 2019 (COVID-19): A Review. JAMA 2020, 324 (8), 782-793. 32648899

53. Cain, D. W.; Cidlowski, J. A., After 62 years of regulating immunity, dexamethasone meets COVID-19. Nature Reviews Immunology 2020, [Epub ahead of print], 1-2. PMC7416654

54. Cauchois, R.; Koubi, M.; Delarbre, D.; Manet, C.; Carvelli, J.; Blasco, V. B.; Jean, R.; Fouche, L.; Bornet, C.; Pauly, V.; Mazodier, K.; Pestre, V.; Jarrot, P.-A.; Dinarello, C. A.; Kaplanski, G., Early IL-1 receptor blockade in severe inflammatory respiratory failure complicating COVID-19. Proceedings of the National Academy of Sciences 2020, 117 (32), 18951-18953. PMC7430998

55. Kimmig, L. M.; Wu, D.; Gold, M.; Pettit, N. N.; Pitrak, D.; Mueller, J.; Husain, A. N.; Mutlu, E. A.; Mutlu, G. M., IL6 inhibition in critically ill COVID-19 patients is associated with increased secondary infections. medRxiv 2020. PMC7491533

56. Campochiaro, C.; Dagna, L., The conundrum of interleukin-6 blockade in COVID-19. The Lancet Rheumatology 2020, 2 (10), E579-580. PMC7428300

57. Behrens, E. M.; Koretzky, G. A., Review: Cytokine Storm Syndrome: Looking Toward the Precision Medicine Era. Arthritis & Rheumatology 2017, 69 (6), 1135-1143. 28217930

58. Santomasso, B.; Bachier, C.; Westin, J.; Rezvani, K.; Shpall, E. J., The other side of CAR T-cell therapy: cytokine release syndrome, neurologic toxicity, and financial burden. American Society of Clinical Oncology Educational Book: 2019; pp. 433-444. 31099694

59. Kotch, C.; Barrett, D.; Teachey, D. T., Tocilizumab for the treatment of chimeric antigen receptor T cell-induced cytokine release syndrome. Expert Review of Clinical Immunology 2019, 15 (8), 813-822. 31219357

60. Giavridis, T.; van der Stegen, S. J. C.; Eyquem, J.; Hamieh, M.; Piersigilli, A.; Sadelain, M., CAR T cell-induced cytokine release syndrome is mediated by macrophages and abated by IL-1 blockade. Nature Medicine 2018, 24 (6), 731-738. PMC6410714

61. Sumegi, J.; Barnes, M. G.; Nestheide, S. V.; Molleran-Lee, S.; Villanueva, J.; Zhang, K.; Risma, K. A.; Grom, A. A.; Filipovich, A. H., Gene expression profiling of peripheral blood mononuclear cells from children with active hemophagocytic lymphohistiocytosis. Blood 2011, 117 (15), e151-e160. PMC3087540

62. Ramos-Casals, M.; Brito-Zeron, P.; Lopez-Guillermo, A.; Khamashta, M. A.; Bosch, X., Adult haemophagocytic syndrome. The Lancet 2014, 383 (9927), 1503-1516. 24290661

63. Kyriazopoulou, E.; Leventogiannis, K.; Norrby-Teglund, A.; Dimopoulos, G.; Pantazi, A.; Orfanos, S. E.; Rovina, N.; Tsangaris, I.; Gkavogianni, T.; Botsa, E.; Chassiou, E.; Kotanidou, A.; Kontouli, C.; Chaloulis, P.; Velissaris, D.; Savva, A.; Cullberg, J.-S.; Akinosoglou, K.; Gogos, C.; Armaganidis, A.; Giamarellos-Bourboulis, E. J.; Hellenic Sepsis Study, G., Macrophage activation-like syndrome: an immunological entity associated with rapid progression to death in sepsis. BMC Med 2017, 15 (1), 172-172. PMC5603161

64. Lerkvaleekul, B.; Vilaiyuk, S., Macrophage activation syndrome: early diagnosis is key. Open Access Rheumatology 2018, 10, 117-128. PMC6124446

65. Mazodier, K.; Marin, V.; Novick, D.; Farnarier, C.; Robitail, S.; Schleinitz, N.; Veit, V.; Paul, P.; Rubinstein, M.; Dinarello, C. A.; Harle, J.-R.; Kaplanski, G., Severe imbalance of IL-18/IL-18BP in patients with secondary hemophagocytic syndrome. Blood 2005, 106 (10), 3483-3489. PMC1895045

66. Lee, J. S.; Park, S.; Jeong, H. W.; Ahn, J. Y.; Choi, S. J.; Lee, H.; Choi, B.; Nam, S. K.; Sa, M.; Kwon, J.-S.; Jeong, S. J.; Lee, H. K.; Park, S. H.; Park, S.-H.; Choi, J. Y.; Kim, S.-H.; Jung, I.; Shin, E.-C., Immunophenotyping of COVID-19 and influenza highlights the role of type I interferons in development of severe COVID-19. Science Immunology 2020, 5 (49), eabd1554. PMC7402635

67. Hu, Z.-J.; Xu, J.; Yin, J.-M.; Li, L.; Hou, W.; Zhang, L.-L.; Zhou, Z.; Yu, Y.-Z.; Li, H.-J.; Feng, Y.-M.; Jin, R.-H., Lower Circulating Interferon-Gamma Is a Risk Factor for Lung Fibrosis in COVID-19 Patients. Frontiers in Immunology 2020, 11 (2348). PMC7550399

68. Lagunas-Rangel, F. A.; Chavez-Valencia, V., High IL-6/IFN-γ ratio could be associated with severe disease in COVID-19 patients. Journal of Medical Virology 2020, 92 (10), 1789-1790. PMC7262117

69. Mendoza, J. L.; Escalante, N. K.; Jude, K. M.; Bellon, J. S.; Su, L.; Horton, T. M.; Tsutsumi, N.; Berardinelli, S. J.; Haltiwanger, R. S.; Piehler, J., Structure of the IFNγ receptor complex guides design of biased agonists. Nature 2019, 1. PMC6561087

70. Lee, I. C.; Huang, Y. H.; Chau, G. Y.; Huo, T. I.; Su, C. W.; Wu, J. C.; Lin, H. C., Serum interferon gamma level predicts recurrence in hepatocellular carcinoma patients after curative treatments. International Journal of Cancer 2013, 133 (12), 2895-2902. 23749461

71. Liu, J.; Gao, L.; Zang, D., Elevated levels of IFN-γ in CSF and serum of patients with amyotrophic lateral sclerosis. PLoS One 2015, 10 (9), e0136937. PMC4557946

72. Song, M.; Ping, Y.; Zhang, K.; Yang, L.; Li, F.; Zhang, C.; Cheng, S.; Yue, D.; Maimela, N. R.; Qu, J.; Liu, S.; Sun, T.; Li, Z.; Xia, J.; Zhang, B.; Wang, L.; Zhang, Y., Low-dose IFNγ induces tumor cell stemness in tumor microenvironment of non-small cell lung cancer. Cancer Research 2019, 79 (14), 3737-3748. 31085700

73. Zhang, D.; Wang, Z.; Wang, L.; Wang, Z.; Wang, H.; Li, G.; Qiao, Z.-Y.; Xu, W.; Wang, H., Highperformance identification of human bladder cancer using a signal self-amplifiable photoacoustic nanoprobe. ACS Applied Materials & Interfaces 2018, 10 (34), 28331-28339. 29989788

74. Hariri, A.; Alipour, K.; Mantri, Y.; Schulze, J. P.; Jokerst, J. V., Deep learning improves contrast in low-fluence photoacoustic imaging. Biomed Opt Express 2020, 11 (6), 3360-3373. PMC7316023

75. Lee, C.; Jeon, M.; Kim, C., 3-Photoacoustic imaging in nanomedicine. In Applications of Nanoscience in Photomedicine, Hamblin, M. R.; Avci, P., Eds. Chandos Publishing: Oxford, 2015; pp 31-47. PMCID

76. Mallidi, S.; Luke, G. P.; Emelianov, S., Photoacoustic imaging in cancer detection, diagnosis, and treatment guidance. Trends Biotechnol 2011, 29 (5), 213-221. PMC3080445

77. Luke, G. P.; Yeager, D.; Emelianov, S. Y., Biomedical Applications of Photoacoustic Imaging with Exogenous Contrast Agents. Annals of Biomedical Engineering 2012, 40 (2), 422-437. 22048668

78. Hilderbrand, S. A.; Weissleder, R., Near-infrared fluorescence: application to in vivo molecular imaging. Current Opinion in Chemical Biology 2010, 14 (1), 71-79. 19879798

79. Changalvaie, B.; Han, S.; Moaseri, E.; Scaletti, F.; Truong, L.; Caplan, R.; Cao, A.; Bouchard, R.; Truskett, T. M.; Sokolov, K. V.; Johnston, K. P., Indocyanine green J aggregates in polymersomes for near-infrared photoacoustic imaging. ACS Applied Materials & Interfaces 2019, 11 (50), 46437-46450. 31804795

80. Wang, L. V., Multiscale photoacoustic microscopy and computed tomography. Nat Photonics 2009, 3 (9), 503-509. PMC2802217

81. Xavierselvan, M.; Singh, M. K. A.; Mallidi, S., In Vivo Tumor Vascular Imaging with Light Emitting Diode-Based Photoacoustic Imaging System. Sensors (Basel, Switzerland) 2020, 20 (16), 4503. PMC7472236

82. Fatima, A.; Kratkiewicz, K.; Manwar, R.; Zafar, M.; Zhang, R.; Huang, B.; Dadashzadeh, N.; Xia, J.; Avanaki, K., Review of cost reduction methods in photoacoustic computed tomography. Photoacoustics 2019, 15, 100137. PMC6693691

83. Ijas, H.; Hakaste, I.; Shen, B.; Kostiainen, M. A.; Linko, V., Reconfigurable DNA origami nanocapsule for pH-controlled encapsulation and display of cargo. ACS Nano 2019, 13 (5), 5959-5967. PMC7076726

84. Turek, V. A.; Chikkaraddy, R.; Cormier, S.; Stockham, B.; Ding, T.; Keyser, U. F.; Baumberg, J. J., Thermo-responsive actuation of a DNA origami flexor. Advanced Functional Materials 2018, 28 (25), 1706410.

85. Selnihhin, D.; Sparvath, S. M.; Preus, S.; Birkedal, V.; Andersen, E. S., Multifluorophore DNA origami beacon as a biosensing platform. ACS Nano 2018, 12 (6), 5699-5708. 29763544

86. Rothemund, P. W. K., Folding DNA to create nanoscale shapes and patterns. Nature 2006, 440 (7082), 297-302. 16541064

87. Castro, C. E.; Kilchherr, F.; Kim, D.-N.; Shiao, E. L.; Wauer, T.; Wortmann, P.; Bathe, M.; Dietz, H., A primer to scaffolded DNA origami. Nature Methods 2011, 8 (3), 221-229. 21358626

88. Liu, M.; Fu, J.; Hejesen, C.; Yang, Y.; Woodbury, N. W.; Gothelf, K.; Liu, Y.; Yan, H., A DNA tweezer-actuated enzyme nanoreactor. Nature Communications 2013, 4 (1), 2127. 23820332

89. Dhakal, S.; Adendorff, M. R.; Liu, M.; Yan, H.; Bathe, M.; Walter, N. G., Rational design of DNA-actuated enzyme nanoreactors guided by single molecule analysis. Nanoscale 2016, 8 (5), 3125-3137. 26788713

90. Noteborn, W. E. M.; Abendstein, L.; Sharp, T. H., One-Pot Synthesis of Defined-Length ssDNA for Multiscaffold DNA Origami. Bioconjugate Chemistry 2021, 32 (1), 94-98. PMC7830112

91. Kim, D.-N.; Kilchherr, F.; Dietz, H.; Bathe, M., Quantitative prediction of 3D solution shape and flexibility of nucleic acid nanostructures. Nucleic Acids Research 2012, 40 (7), 2862-2868. PMC3326316

92. Pettersen, E. F.; Goddard, T. D.; Huang, C. C.; Couch, G. S.; Greenblatt, D. M.; Meng, E. C.; Ferrin, T.E., UCSF Chimera—a visualization system for exploratory research and analysis. Journal of computational chemistry 2004, 25 (13), 1605-12.

93. Borg, R. E.; Hatamimoslehabadi, M.; Bellinger, S.; La, J.; Mithila, F.; Yelleswarapu, C.; Rochford, J., Photophysical and photoacoustic properties of π-extended curcumin dyes. Effects of the terminal dimethylamino electron-donor and the bridging aryl ring. Photochem Photobiol 2019, 95 (1), 280-292. PMC6328350

94. Idili, A.; Vallee-Belisle, A.; Ricci, F., Programmable pH-Triggered DNA Nanoswitches. J Am Chem Soc 2014, 136 (16), 5836-5839.

95. Thiel, D.; Le Du, M.; Walter, R.; D'Arcy, A.; Chene, C.; Fountoulakis, M.; Garotta, G.; Winkler, F.; Ealick, S., Observation of an unexpected third receptor molecule in the crystal structure of human interferon-γ receptor complex. Structure 2000, 8 (9), 927-936. 10986460

96. Taga, T.; Kishimoto, T., gp130 and the interleukin-6 family of cytokines. Annual Review of Immunology 1997, 15 (1), 797-819. 9143707

97. Fields, J. K.; Gunther, S.; Sundberg, E. J., Structural Basis of IL-1 Family Cytokine Signaling. Frontiers in Immunology 2019, 10 (1412). PMC6596353

98. Jostock, T.; Mullberg, J.; Ozbek, S.; Atreya, R.; Blinn, G.; Voltz, N.; Fischer, M.; Neurath, M. F.; Rose-John, S., Soluble gp130 is the natural inhibitor of soluble interleukin-6 receptor transsignaling responses. European Journal of Biochemistry 2001, 268 (1), 160-167. 11121117

99. Meredith, G. D.; Wu, H. Y.; Allbritton, N. L., Targeted protein functionalization using His-tags. Bioconjugate Chemistry 2004, 15 (5), 969-982. 15366949

100. Rosen, C. B.; Kodal, A. L. B.; Nielsen, J. S.; Schaffert, D. H.; Scavenius, C.; Okholm, A. H.; Voigt, N. V.; Enghild, J. J.; Kjems, J.; Toning, T.; Gothelf, K. V., Template-directed covalent conjugation of DNA to native antibodies, transferrin and other metal-binding proteins. Nature Chemistry 2014, 6 (9), 804-809. 25143216

101. Schroder, K.; Hertzog, P. J.; Ravasi, T.; Hume, D. A., Interferon-γ: an overview of signals, mechanisms and functions. Journal of Leukocyte Biology 2004, 75 (2), 163-189. 14525967

102. Graves, B. J.; Hatada, M. H.; Hendrickson, W. A.; Miller, J. K.; Madison, V. S.; Satow, Y., Structure of interleukin 1α at 2.7-A resolution. Biochemistry 1990, 29 (11), 2679-2684.

103. Chang, H.-K.; Mohan, S. K.; Chin, Y. J. B. N. a., 1H, 13C and 1⁵N backbone and side chain resonance assignments of human interleukin 1α. Biomolecular NMR Assignments 2010, 4, 59-60.

104. Dinarello, C. A., A clinical perspective of IL-1β as the gatekeeper of inflammation. Eur. J Immunol. 2011, 41 (5), 1203-1217. 21523780

105. Anastassacos, F. M.; Zhao, Z.; Zeng, Y.; Shih, W. M., Glutaraldehyde cross-linking of oligolysines coating DNA origami greatly reduces susceptibility to nuclease degradation. J Am Chem Soc 2020, 142 (7), 3311-3315. 32011869

106. Lu, X.; Zhang, K., PEGylation of therapeutic oligonucletides: From linear to highly branched PEG architectures. Nano Res 2018, 11 (10), 5519-5534. PMC6366847

107. Wang, S.-T.; Gray, M. A.; Xuan, S.; Lin, Y.; Byrnes, J.; Nguyen, A. I.; Todorova, N.; Stevens, M. M.; Bertozzi, C. R.; Zuckermann, R. N.; Gang, O., DNA origami protection and molecular interfacing through engineered sequence-defined peptoids. Proceedings of the National Academy of Sciences 2020, 201919749. PMC7104344

108. Auvinen, H.; Zhang, H.; Nonappa; Kopilow, A.; Niemela, E. H.; Nummelin, S.; Correia, A.; Santos, H. A.; Linko, V.; Kostiainen, M. A., Protein coating of DNA nanostructures for enhanced stability and immunocompatibility. Advanced Healthcare Materials 2017, 6 (18), 1700692. 28738444

109. Gerling, T.; Kube, M.; Kick, B.; Dietz, H., Sequence-programmable covalent bonding of designed DNA assemblies. Science Advances 2018, 4 (8), eaau1157. PMC6097813

110. Cassinelli, V.; Oberleitner, B.; Sobotta, J.; Nickels, P.; Grossi, G.; Kempter, S.; Frischmuth, T.; Liedl, T.; Manetto, A., One-step formation of “chain-armor”-stabilized DNA nanostructures. Angewandte Chemie International Edition English 2015, 54 (27), 7795-7798. 25980669

111. Weber, J.; Beard, P. C.; Bohndiek, S. E., Contrast agents for molecular photoacoustic imaging. Nature methods. 2016, 13 (8), 639-650.

112. Liu, R.; Tang, J.; Xu, Y.; Zhou, Y.; Dai, Z., Nano-sized Indocyanine Green J-aggregate as a One-component Theranostic Agent. Nanotheranostics. 2017, 1 (4), 430-439.

113. Holzer, W.; Mauerer, M.; Penzkofer, A.; Szeimies, R.-M.; Abels, C.; Landthaler, M.; Baumler, W., Photostability and thermal stability of indocyanine green. Journal of Photochemistry and Photobiology B: Biology 1998, 47 (2-3), 155-164. 10093915

114. Philip, R.; Penzkofer, A.; Baumler, W.; Szeimies, R.; Abels, C., Absorption and fluorescence spectroscopic investigation of indocyanine green. Journal of Photochemistry and Photobiology A: Chemistry 1996, 96 (1-3), 137-148.

115. Zweck, J.; Penzkofer, A., Microstructure of indocyanine green J-aggregates in aqueous solution. Chemical Physics 2001, 269 (1), 399-409.

116. Guan, T.; Shang, W.; Li, H.; Yang, X.; Fang, C.; Tian, J.; Wang, K., From Detection to Resection: Photoacoustic Tomography and Surgery Guidance with Indocyanine Green Loaded Gold Nanorod@liposome Core-Shell Nanoparticles in Liver Cancer. Bioconjugate Chemistry 2017, 28 (4), 1221-1228. 28345887

117. Miranda, D.; Huang, H.; Kang, H.; Zhan, Y.; Wang, D.; Zhou, Y.; Geng, J.; Kilian, H. I.; Stiles, W.; Razi, A.; Ortega, J.; Xia, J.; Choi, H. S.; Lovell, J. F., Highly-Soluble Cyanine J-aggregates Entrapped by Liposomes for In Vivo Optical Imaging around 930 nm. Theranostics. 2019, 9 (2), 381-390.

118. Phillips, D.; Dhami, S.; Ostler, R.; Petrasek, Z., The Dimerisation of Phthalocyanines. Progress in Reaction Kinetics and Mechanism 2003, 28 (4), 299-420.

119. Fagnani, D. E.; Sotuyo, A.; Castellano, R. K., 1.06 π-π Interactions. In Comprehensive Supramolecular Chemistry II, Atwood, J. L., Ed. Elsevier: Oxford, 2017; pp 121-148. PMCID

120. Gray, M.; Goodman, A. J.; Carroll, J. B.; Bardon, K.; Markey, M.; Cooke, G.; Rotello, V. M., Model Systems for Flavoenzyme Activity: Interplay of Hydrogen Bonding and Aromatic Stacking in Cofactor Redox Modulation. Organic Letters 2004, 6 (3), 385-388. 14748599

121. Nowick, J. S.; Ballester, P.; Ebmeyer, F.; Rebek, J., Convergent functional groups. 9. Complexation in new molecular clefts. J Am Chem Soc 1990, 112 (24), 8902-8906.

122. Mallidi, S.; Larson, T.; Tam, J.; Joshi, P. P.; Karpiouk, A.; Sokolov, K.; Emelianov, S., Multiwavelength photoacoustic imaging and plasmon resonance coupling of gold nanoparticles for selective detection of cancer. Nano Letters 2009, 9 (8), 2825-2831. PMC2898720

123. Gould, T.; Wang, Q.; Pfefer, T. J., Optical-thermal light-tissue interactions during photoacoustic breast imaging. Biomed Opt Express 2014, 5 (3), 832-847. PMC3959836

124. Bag, D. S.; Kumar, V. P.; Maiti, S., Chemical modification of LDPE film. Journal of Applied Polymer Science 1999, 71 (7), 1041-1048.

125. Rasmussen, J. R.; Bergbreiter, D. E.; Whitesides, G. M., Location and mobility of functional groups at the surface of oxidized, low-density polyethylene film. J Am Chem Soc 1977, 99 (14), 4746-4756.

126. Goddard, J. M.; Hotchkiss, J. H., Tailored functionalization of low-density polyethylene surfaces. Journal of Applied Polymer Science 2008, 108 (5), 2940-2949.

127. Vogt, W. C.; Jia, C.; Wear, K. A.; Garra, B. S.; Joshua Pfefer, T., Biologically relevant photoacoustic imaging phantoms with tunable optical and acoustic properties. Journal of Biomedical Optics 2016, 21 (10), 101405-101405. PMC4756225

128. Ramirez, K.; Quesada-Yamasaki, D.; Fornaguera-Trias, J., A Protocol to Perform Systemic Lipopolysacharide (LPS) Challenge in Rats. Odovtos—International Journal of Dental Sciences. 2019, 21 (1), 53-66.

(A1) Biechele, P.; Busse, C.; Solle, D.; Scheper, T.; Reardon, K. Sensor systems for bioprocess monitoring. Engineering in Life Sciences 2015, 15 (5), 469-488.

(A2) Plaxco, K. W.; Soh, H. T. Switch-based biosensors: a new approach towards real-time, in vivo molecular detection. Trends Biotechnol. 2011, 29 (1), 1-5.

(A3) Bagalad, B. S.; Mohankumar, K.; Madhushankari, G.; Donoghue, M.; Kuberappa, P. H. Diagnostic accuracy of salivary creatinine, urea, and potassium levels to assess dialysis need in renal failure patients. Dent. Res. J. 2017, 14 (1), 13-18.

(A4) Stengaard, C.; Sørensen, J. T.; Ladefoged, S. A.; Lassen, J. F.; Rasmussen, M. B.; Pedersen, C. K.; Ayer, A.; Bøtker, H. E.; Terkelsen, C. J.; Thygesen, K. The potential of optimizing prehospital triage of patients with suspected acute myocardial infarction using highsensitivity cardiac troponin T and copeptin. Biomarkers 2017, 22, 351-360.

(A5) Schoenborn, J. R.; Wilson, C. B. Regulation of interferon γ during innate and adaptive immune responses. Adv. Immunol. 2007, 96, 41-101.

(A6) Miller, C. H.; Maher, S. G.; Young, H. A. Clinical use of interferon γ. Ann. N. Y. Acad. Sci. 2009, 1182 (1), 69-79.

(A7) Bracaglia, C.; Marafon, D. P.; Caiello, I.; de Graaf, K.; Guilhot, F.; Ferlin, W.; Davi, S.; Schulert, G.; Ravelli, A.; Grom, A.; Nelson, R.; de Min, C.; De Benedetti, F. High levels of interferon-gamma (IFNγ) in macrophage activation syndrome (MAS) and CXCL9 levels as a biomarker for IFNγ production in MAS. Pediatric Rheumatology 2015, 13 (1), O84-O85.

(A8) Wen, A.; Qu, X.-H.; Zhang, K.-N.; Leng, E.-L.; Ren, Y.; Wu, X.-M. Evaluation of interferon-gamma release assays in extrasanguinous body fluids for diagnosing tuberculosis: A systematic review and metaanalysis. Life Sci. 2018, 197, 140-146.

(A9) Topalian, S. L.; Taube, J. M.; Anders, R. A.; Pardoll, D. M. Mechanism-driven biomarkers to guide immune checkpoint blockade in cancer therapy. Nat. Rev. Cancer 2016, 16, 275-287.

(A10) Parker, B. S.; Rautela, J.; Hertzog, P. J. Antitumour actions of interferons: implications for cancer therapy. Nat. Rev. Cancer 2016, 16 (3), 131-144.

(A11) Holmin, S.; Höjeberg, B. In situ detection of intracerebral cytokine expression after human brain contusion. Neurosci. Lett. 2004, 369 (2), 108-114.

(A12) Reddy, S.; Karanam, M.; Krissansen, G.; Nitschke, K.; Neve, J.; Poole, C. A.; Ross, J. M. Temporal Relationship Between Immune Cell Influx and the Expression of Inducible Nitric Oxide Synthase, Interleukin-4 and Interferon-γ in Pancreatic Islets of NOD Mice Following Adoptive Transfer of Diabetic Spleen Cells. Histochem. J. 2000, 32 (4), 195-206.

(A13) Yan, Y.; Kolachala, V.; Dalmasso, G.; Nguyen, H.; Laroui, H.; Sitaraman, S. V.; Merlin, D. Temporal and Spatial Analysis of Clinical and Molecular Parameters in Dextran Sodium Sulfate Induced Colitis. PLoS One 2009, 4 (6), e6073-e6081.

(A14) Zha, Z.; Bucher, F.; Nejatfard, A.; Zheng, T.; Zhang, H.; Yea, K.; Lerner, R. A. Interferon-γ is a master checkpoint regulator of cytokine-induced differentiation. Proc. Natl. Acad. Sci. U.S.A. 2017, 114 (33), E6867-E6874.

(A15) Kurane, I.; Innis, B. L.; Nimmannitya, S.; Nisalak, A.; Meager, A.; Janus, J.; Ennis, F. A. Activation of T lymphocytes in dengue virus infections. High levels of soluble interleukin 2 receptor, soluble CD4, soluble CD8, interleukin 2, and interferon-gamma in sera of children with dengue. J. Clin. Invest. 1991, 88 (5), 1473-1480.

(A16) Monastero, R. N.; Pentyala, S. Cytokines as Biomarkers and Their Respective Clinical Cutoff Levels. Int. J. Inflammation 2017, 2017, 1-11.

(A17) The International Chronic Granulomatous Disease Cooperative Study Group. A Controlled Trial of Interferon Gamma to Prevent Infection in Chronic Granulomatous Disease. N. Engl. J. Med. 1991, 324 (8), 509-516.

(A18) Ikeda, H.; Old, L. J.; Schreiber, R. D. The roles of IFNγ in protection against tumor development and cancer immunoediting. Cytokine Growth Factor Rev. 2002, 13 (2), 95-109.

(A19) Jonasch, E.; Haluska, F. G. Interferon in oncological practice: review of interferon biology, clinical applications, and toxicities. Oncologist 2001, 6 (1), 34-55.

(A20) Thom, A. K.; Alexander, H. R.; Andrich, M. P.; Barker, W.; Rosenberg, S. A.; Fraker, D. L. Cytokine levels and systemic toxicity in patients undergoing isolated limb perfusion with high-dose tumor necrosis factor, interferon gamma, and melphalan. J. Clin. Oncol. 1995, 13 (1), 264-273.

(A21) Razaghi, A.; Owens, L.; Heimann, K. Review of the recombinant human interferon gamma as an immunotherapeutic: Impacts of production platforms and glycosylation. J. Biotechnol. 2016, 240, 48-60.

(A22) Zaidi, M. R.; Merlino, G. The two faces of interferon-γ in cancer. Clin. Cancer Res. 2011, 17 (19), 6118-6124.

(A23) Mojic, M.; Takeda, K.; Hayakawa, Y. The Dark Side of IFN-γ: Its Role in Promoting Cancer Immunoevasion. Int. J. Mol. Sci. 2018, 19 (1), 89-102.

(A24) Liu, G.; Qi, M.; Hutchinson, M. R.; Yang, G.; Goldys, E. M. Recent advances in cytokine detection by immunosensing. Biosens. Bioelectron. 2016, 79, 810-821.

(A25) Vestergaard, M.; Kerman, K.; Tamiya, E. An overview of labelfree electrochemical protein sensors. Sensors 2007, 7 (12), 3442-3458.

(A26) Xia, J.; Song, D.; Wang, Z.; Zhang, F.; Yang, M.; Gui, R.; Xia, L.; Bi, S.; Xia, Y.; Li, Y.; Xia, L. Single electrode biosensor for simultaneous determination of interferon gamma and lysozyme. Biosens. Bioelectron. 2015, 68, 55-61.

(A27) Madondo, M. T.; Quinn, M.; Plebanski, M. Low dose cyclophosphamide: mechanisms of T cell modulation. Cancer Treat. Rev. 2016, 42, 3-9.

(A28) Jackman, R. P.; Utter, G. H.; Heitman, J. W.; Hirschkorn, D. F.; Law, J. P.; Gefter, N.; Busch, M. P.; Norris, P. J. Effects of Blood Sample Age at Time of Separation on Measured Cytokine Concentrations in Human Plasma. Clin. Vaccine Immunol. 2011, 18 (2), 318-326.

(A29) Zhou, X.; Fragala, M. S.; McElhaney, J. E.; Kuchel, G. A. Conceptual and methodological issues relevant to cytokine and inflammatory marker measurements in clinical research. Curr. Opin. Clin. Nutr. Metab. Care 2010, 13 (5), 541-547.

(A30) Seiler, W.; Muller, H.; Hiemke, C. Interleukin-6 in plasma collected with an indwelling cannula reflects local, not systemic, concentrations. Clinical Chemistry 1994, 40 (9), 1778-1779.

(A31) Zhang, K.; Baratta, M. V.; Liu, G.; Frank, M. G.; Leslie, N. R.; Watkins, L. R.; Maier, S. F.; Hutchinson, M. R.; Goldys, E. M. A novel platform for in vivo detection of cytokine release within discrete brain regions. Brain, Behav., Immun. 2018, 71, 18-22.

(A32) McKeague, M.; DeRosa, M. C. Challenges and opportunities for small molecule aptamer development. J. Nucleic Acids 2012, 2012, 1-21.

(A33) Kim, J.; Lee, D.; Jung, U.; Kim, C. Photoacoustic imaging platforms for multimodal imaging. Ultrasonography 2015, 34 (2), 88-97.

(A34) Mallidi, S.; Luke, G. P.; Emelianov, S. Photoacoustic imaging in cancer detection, diagnosis, and treatment guidance. Trends Biotechnol. 2011, 29 (5), 213-221.

(A35) Kim, C.; Favazza, C.; Wang, L. V. In vivo photoacoustic tomography of chemicals: high-resolution functional and molecular optical imaging at new depths. Chem. Rev. 2010, 110 (5), 2756-2782.

(A36) Hariri, A.; Lemaster, J.; Wang, J.; Jeevarathinam, A. S.; Chao, D. L.; Jokerst, J. V. The characterization of an economic and portable LED-based photoacoustic imaging system to facilitate molecular imaging. Photoacoustics 2018, 9, 10-20.

(A37) Levi, J.; Kothapalli, S. R.; Ma, T.-J.; Hartman, K.; Khuri-Yakub, B. T.; Gambhir, S. S. Design, synthesis, and imaging of an activatable photoacoustic probe. J. Am. Chem. Soc. 2010, 132 (32), 11264-11269.

(A38) Chatni, M. R.; Xia, J.; Sohn, R.; Maslov, K.; Guo, Z.; Zhang, Y.; Wang, K.; Xia, Y.; Anastasio, M.; Arbeit, J.; Wang, L. V. Tumor glucose metabolism imaged in vivo in small animals with whole-body photoacoustic computed tomography. J. Biomed. Opt. 2012, 17 (7), 0760121-0760127.

(A39) Ng, K. K.; Shakiba, M.; Huynh, E.; Weersink, R. A.; Roxin, A.; Wilson, B. C.; Zheng, G. Stimuli-responsive photoacoustic nanoswitch for in vivo sensing applications. ACS Nano 2014, 8 (8), 8363-8373.

(A40) Cash, K. J.; Li, C.; Xia, J.; Wang, L. V.; Clark, H. A. Optical drug monitoring: photoacoustic imaging of nanosensors to monitor therapeutic lithium in vivo. ACS Nano 2015, 9 (2), 1692-1698.

(A41) Lee, C. H.; Folz, J.; Zhang, W.; Jo, J.; Tan, J. W.; Wang, X.; Kopelman, R. Ion-selective nanosensor for photoacoustic and fluorescence imaging of potassium. Anal. Chem. 2017, 89 (15), 7943-7949.

(A42) Ariga, K.; Jackman, J. A.; Cho, N. J.; Hsu, S. H.; Shrestha, L. K.; Mori, T.; Takeya, J. Nanoarchitectonic Based Material Platforms for Environmental and Bioprocessing Applications. Chem. Rec. 2018, 18, 1-23.

(A43) Hirasawa, T.; Iwatate, R. J.; Kamiya, M.; Okawa, S.; Urano, Y.; Ishihara, M. Multispectral photoacoustic imaging of tumours in mice injected with an enzyme-activatable photoacoustic probe. J. Opt. 2017, 19 (1), 014002-014017.

(A44) Ruckh, T. T.; Clark, H. A. Implantable nanosensors: toward continuous physiologic monitoring. Anal. Chem. 2014, 86 (3), 1314-1323.

(A45) Rong, G.; Corrie, S. R.; Clark, H. A. In vivo biosensing: progress and perspectives. ACS Sensors 2017, 2 (3), 327-338.

(A46) McQuade, D. T.; Hegedus, A. H.; Swager, T. M. Signal amplification of a “turn-on” sensor: Harvesting the light captured by a conjugated polymer. J. Am. Chem. Soc. 2000, 122 (49), 12389-12390.

(A47) Zhang, X.-F.; Xi, Q.; Zhao, J. Fluorescent and triplet state photoactive J-type phthalocyanine nano assemblies: controlled formation and photosensitizing properties. J. Mater. Chem. 2010, 20 (32), 6726-6733.

(A48) Dinarello, C. A.; Fantuzzi, G. Interleukin-18 and host defense against infection. J. Infect. Dis. 2003, 187, S370-S384.

(A49) Bušek, P.; [symbol] , R.; Šedo, A. Dipeptidyl peptidase IV activity and/or structure homologues (DASH) and their substrates in cancer. Int. J. Biochem. Cell Biol. 2004, 36 (3), 408-421.

(A50) Tainaka, K.; Sakaguchi, R.; Hayashi, H.; Nakano, S.; Liew, F. F.; Morii, T. Design strategies of fluorescent biosensors based on biological macromolecular receptors. Sensors 2010, 10 (2), 1355-1376.

(A51) Alivisatos, A. P.; Andrews, A. M.; Boyden, E. S.; Chun, M.; Church, G. M.; Deisseroth, K.; Donoghue, J. P.; Fraser, S. E.; Lippincott-Schwartz, J.; Looger, L. L. Nanotools for neuroscience and brain activity mapping. ACS Nano 2013, 7 (3), 1850-1866.

(A52) Kallenbach, N. R.; Ma, R.-I.; Seeman, N. C. An immobile nucleic acid junction constructed from oligonucleotides. Nature 1983, 305 (5937), 829-831.

(A53) Song, C.; Wang, Z.-G.; Ding, B. Design, Fabrication, and Applications of DNA Nanomachines. In DNA Nanotechnology; Springer, 2013; pp 225-261.

(A54) Nesterova, I. V.; Erdem, S. S.; Pakhomov, S.; Hammer, R. P.; Soper, S. A. Phthalocyanine dimerization-based molecular beacons using near-IR fluorescence. J. Am. Chem. Soc. 2009, 131 (7), 2432-2433.

(A55) Mak, T. W.; Saunders, M. E. The immune response: basic and clinical principles; Academic Press, 2005; pp 471-474.

(A56) Thiel, D.; Le Du, M.; Walter, R.; D'Arcy, A.; Chene, C.; Fountoulakis, M.; Garotta, G.; Winkler, F.; Ealick, S. Observation of an unexpected third receptor molecule in the crystal structure of human interferon-γ receptor complex. Structure 2000, 8 (9), 927-936.

(A57) Walter, M. R.; Windsor, W. T.; Nagabhushan, T. L.; Lundell, D. J.; Lunn, C. A.; Zauodny, P. J.; Narula, S. K. Crystal structure of a complex between interferon-γ and its soluble high-affinity receptor. Nature 1995, 376 (6537), 230-235.

(A58) Goltry, S.; Hallstrom, N.; Clark, T.; Kuang, W.; Lee, J.; Jorcyk, C.; Knowlton, W. B.; Yurke, B.; Hughes, W. L.; Graugnard, E. DNA topology influences molecular machine lifetime in human serum. Nanoscale 2015, 7 (23), 10382-10390.

(A59) Schmied, J. J.; Raab, M.; Forthmann, C.; Pibiri, E.; Wunsch, B.; Dammeyer, T.; Tinnefeld, P. DNA origami-based standards for quantitative fluorescence microscopy. Nat. Protoc. 2014, 9 (6), 1367-1391.

(A60) Stellwagen, N. C. Electrophoresis of DNA in agarose gels, polyacrylamide gels and in free solution. Electrophoresis 2009, 30 (51), S188-S195.

(A61) Karlsson, R. SPR for molecular interaction analysis: a review of emerging application areas. J. Mol. Recognit. 2004, 17 (3), 151-161.

(A62) Önell, A.; Andersson, K. Kinetic determinations of molecular interactions using Biacore-minimum data requirements for efficient experimental design. J. Mol. Recognit. 2005, 18 (4), 307-317.

(A63) Bach, E. A.; Aguet, M.; Schreiber, R. D. The IFNγ receptor: a paradigm for cytokine receptor signaling. Annu. Rev. Immunol. 1997, 15 (1), 563-591.

(A64) Langer, J. A.; Cutrone, E. C.; Kotenko, S. The Class II cytokine receptor (CRF2) family: overview and patterns of receptor-ligand interactions. Cytokine Growth Factor Rev. 2004, 15 (1), 33-48.

(A65) Marsters, S. A.; Pennica, D.; Bach, E.; Schreiber, R. D.; Ashkenazi, A. Interferon gamma signals via a high-affinity multisubunit receptor complex that contains two types of polypeptide chain. Proc. Natl. Acad. Sci. U.S.A. 1995, 92 (12), 5401-5405.

(A66) Mikulecký, P.; Cerný, J.; Biedermannová, L.; Petroková, H.; Kuchař, M.; Vondrášek, J.; Malý, P.; Šebo, P.; Schneider, B. Increasing Affinity of Interferon-Receptor 1 to Interferon-by Computer-Aided Design. BioMed Res. Int. 2013, 2013, 1017-1025.

(A67) Aarreberg, L. D.; Wilkins, C.; Ramos, H. J.; Green, R.; Davis, M. A.; Chow, K.; Gale, M. Interleukin-1β Signaling in Dendritic Cells Induces Antiviral Interferon Responses. mBio 2018, 9 (2), e00342-e00358.

(A68) Claessens, C. G.; Hahn, U.; Tones, T. Phthalocyanines: From outstanding electronic properties to emerging applications. Chem. Rec. 2008, 8 (2), 75-97.

(A69) Attia, A. B. E.; Balasundaram, G.; Driessen, W.; Ntziachristos, V.; Olivo, M. Phthalocyanine photosensitizers as contrast agents for in vivo photoacoustic tumor imaging. Biomed. Opt. Express 2015, 6 (2), 591-598.

(A70) Wilson, K. E.; Bachawal, S. V.; Abou-Elkacem, L.; Jensen, K.; Machtaler, S.; Tian, L.; Willmann, J. K. Spectroscopic photoacoustic molecular imaging of breast cancer using a B7-H3-targeted ICG contrast agent. Theranostics 2017, 7 (6), 1463-1476.

(A71) Rurack, K.; Spieles, M. Fluorescence quantum yields of a series of red and near-infrared dyes emitting at 600-1000 nm. Anal. Chem. 2011, 83 (4), 1232-1242.

(A72) Anula, H.; Berlin, J. C.; Wu, H.; Li, Y.-S.; Peng, X.; Kenney, M. E.; Rodgers, M. A. Synthesis and photophysical properties of silicon phthalocyanines with axial siloxy ligands bearing alkylamine termini. J. Phys. Chem. A 2006, 110 (15), 5215-5223.

(A73) Owens, J. W.; Robins, M. Phthalocyanine photophysics and photosensitizer efficiency on human embryonic lung fibroblasts. J. Porphyrins Phthalocyanines 2001, 05 (05), 460-464.

(A74) Vo-Dinh, T. Biomedical photonics handbook: biomedical diagnostics; CRC press, 2014; pp 49-50.

(A75) Ferencz, A.; Neher, D.; Schulze, M.; Wegner, G.; Viaene, L.; De Schryver, F. Synthesis and spectroscopic properties of phthalocyanine dimers in solution. Chem. Phys. Lett. 1995, 245 (1), 23-29.

(A76) Heyne, B. Self-assembly of organic dyes in supramolecular aggregates. Photochemical & Photobiological Sciences 2016, 15 (9), 1103-1114.

(A77) Mallidi, S.; Kim, S.; Karpiouk, A.; Joshi, P. P.; Sokolov, K.; Emelianov, S. Visualization of molecular composition and functionality of cancer cells using nanoparticle-augmented ultrasound-guided photoacoustics. Photoacoustics 2015, 3 (1), 26-34.

(A78) Fu, Q.; Zhu, R.; Song, J.; Yang, H.; Chen, X. Photoacoustic Imaging: Contrast Agents and Their Biomedical Applications. Adv. Mater. 2018, 31 (6), 1805875-1805906.

(A79) Lee, I. C.; Huang, Y. H.; Chau, G. Y.; Huo, T. I.; Su, C. W.; Wu, J. C.; Lin, H. C. Serum interferon gamma level predicts recurrence in hepatocellular carcinoma patients after curative treatments. Int. J. Cancer 2013, 133 (12), 2895-2902.

(A80) Liu, J.; Gao, L.; Zang, D. Elevated levels of IFN-γ in CSF and serum of patients with amyotrophic lateral sclerosis. PLoS One 2015, 10 (9), e0136937-e0136948.

(A81) Mei, Q.; Wei, X.; Su, F.; Liu, Y.; Youngbull, C.; Johnson, R.; Lindsay, S.; Yan, H.; Meldrum, D. Stability of DNA origami nanoarrays in cell lysate. Nano Lett. 2011, 11 (4), 1477-1482.

(A82) Cassinelli, V.; Oberleitner, B.; Sobotta, J.; Nickels, P.; Grossi, G.; Kempter, S.; Frischmuth, T.; Liedl, T.; Manetto, A. One Step Formation of “Chain Armor” Stabilized DNA Nanostructures. Angew. Chem., Int. Ed. 2015, 54 (27), 7795-7798.

(A83) Ponnuswamy, N.; Bastings, M. M.; Nathwani, B.; Ryu, J. H.; Chou, L. Y.; Vinther, M.; Li, W. A.; Anastassacos, F. M.; Mooney, D. J.; Shih, W. M. Oligolysine-based coating protects DNA nanostructures from low-salt denaturation and nuclease degradation. Nat. Commun. 2017, 8, 15654-15663.

(A84) Ramakrishnan, S.; Ijäs, H.; Linko, V.; Keller, A. Structural stability of DNA origami nanostructures under application-specific conditions. Comput. Struct. Biotechnol. 2018, 16, 342-349.

(B3) Berraondo, P.; Sanmamed, M. F.; Ochoa, M. C.; Etxeberria, I.; Aznar, M. A.; Pérez-Gracia, J. L.; Rodríguez-Ruiz, M. E.; Ponz-Sarvise, M.; Castañón, E.; Melero, I., Cytokines in clinical cancer immunotherapy. British Journal of Cancer 2019, 120 (1), 6-15. PMC6325155

(B4) Jamilloux, Y.; Henry, T.; Belot, A.; Viel, S.; Fauter, M.; El Jammal, T.; Walzer, T.; François, B.; Sève, P., Should we stimulate or suppress immune responses in COVID-19? Cytokine and anti-cytokine interventions. Autoimmun Rev 2020, 19 (7), 102567-102567. PMC7196557

(B5) Chulpanova, D. S.; Kitaeva, K. V.; Green, A. R.; Rizvanov, A. A.; Solovyeva, V. V., Molecular Aspects and Future Perspectives of Cytokine-Based Anti-cancer Immunotherapy. Front Cell Dev Biol 2020, 8 (402), 402. PMC7283917

(B6) Ji, M. J.; Ryu, H. J.; Hong, J. H., Synovial Fluid of Patient With Rheumatoid Arthritis Enhanced Osmotic Sensitivity Through the Cytotoxic Edema Module in Synoviocytes. Front Cell Dev Biol 2021, 9 (2277), 700879. PMC8438158

(B7) Siebert, S.; Tsoukas, A.; Robertson, J.; McInnes, I., Cytokines as Therapeutic Targets in Rheumatoid Arthritis and Other Inflammatory Diseases. Pharmacological Reviews 2015, 67 (2), 280-309.

(B8) Li, Y.; Yuan, L.; Yang, J.; Lei, Y.; Zhang, H.; Xia, L.; Shen, H.; Lu, J., Changes in Serum Cytokines May Predict Therapeutic Efficacy of Tofacitinib in Rheumatoid Arthritis. Mediators of Inflammation, 2019, 5617431. PMC6855066

(B9) Uno, K.; Yoshizaki, K.; Iwahashi, M.; Yamana, J.; Yamana, S.; Tanigawa, M.; Yagi, K., Pretreatment Prediction of Individual Rheumatoid Arthritis Patients' Response to Anti-Cytokine Therapy Using Serum Cytokine/Chemokine/Soluble Receptor Biomarkers. PLOS ONE 2015, 10 (7), e0132055. PMC4503565

(B10) Kim, J. W.; Kong, J.-S.; Lee, S.; Yoo, S.-A.; Koh, J. H.; Jin, J.; Kim, W.-U., Angiogenic cytokines can reflect the synovitis severity and treatment response to biologics in rheumatoid arthritis. Experimental & Molecular Medicine 2020, 52 (5), 843-853. PMC7272442

(B19) Aziz, N., Measurement of Circulating Cytokines and Immune-Activation Markers by Multiplex Technology in the Clinical Setting: What Are We Really Measuring? For Immunopathol Dis Therap 2015, 6 (1-2), 19-22. PMC5345938

(B20) Venkatesha, S.; Dudics, S.; Acharya, B.; Moudgil, K., Cytokine-Modulating Strategies and Newer Cytokine Targets for Arthritis Therapy. International Journal of Molecular Sciences 2014, 16 (1), 887-906. PMC4307281

(B21) Sacerdote, P.; Franchi, S.; Moretti, S.; Castelli, M.; Procacci, P.; Magnaghi, V.; Panerai, A. E., Cytokine Modulation is Necessary for Efficacious Treatment of Experimental Neuropathic Pain. Journal of Neuroimmune Pharmacology 2013, 8 (1), 202-211.

(B22) Ren, H.; Han, R.; Chen, X.; Liu, X.; Wan, J.; Wang, L.; Yang, X.; Wang, J., Potential therapeutic targets for intracerebral hemorrhage-associated inflammation: An update. J Cereb Blood Flow Metab 2020, 40 (9), 1752-1768. PMC7446569

(B23) Yamamoto, T.; Umegae, S.; Kitagawa, T.; Matsumoto, K., Systemic and Local Cytokine Production in Quiescent Ulcerative Colitis and Its Relationship to Future Relapse: A Prospective Pilot Study. Inflammatory Bowel Diseases 2005, 11 (6), 589-596.

(B24) Clay, T. M.; Hobeika, A. C.; Mosca, P. J.; Lyerly, H. K.; Morse, M. A., Assays for monitoring cellular immune responses to active immunotherapy of cancer. Clinical Cancer Research 2001, 7 (5), 1127-1135. 11350875

(B107) Williams, R. O., Collagen-induced arthritis as a model for rheumatoid arthritis. In Tumor Necrosis Factor, Springer: 2004; pp 207-216. PMCID

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. A system for multiplexed photoacoustic detection of cytokines, the system comprising:
a plurality of nucleic acid-based nanosensors each comprising at least two sensor arms, each arm of the at least two sensor arms comprising at least one photoacoustic reporter chromophore and a cytokine receptor, each arm being configured, in the presence of a detected cytokine in a fluid, to induce stacking of the at least one photoacoustic reporter chromophore with at least one photoacoustic reporter chromophore in another one of the at least two sensor arms, thereby increasing a photoacoustic signal emitted by the nucleic acid based nanosensor, and each arm being further configured to induce unstacking of the at least one photoacoustic reporter chromophore when the cytokine is not present, thereby decreasing the photoacoustic signal emitted;
the plurality of nucleic-acid based nanosensors comprising cytokine receptors for at least two different types of cytokines; and
a photoacoustic measurement system configured to detect the photoacoustic signals emitted by the nucleic acid based nanosensors and to thereby permit distinguishing of a differential photoacoustic signal indicating a stacked or unstacked state of the at least one photoacoustic reporter chromophore of the nucleic acid based nanosensors, for each of the at least two different types of cytokines.

2. (canceled)

3. The system of claim 1, wherein the nucleic-acid based nanosensors are configured to detect two or more of: IFNγ, IL-6, and IL-1.

4. (canceled)

5. The system of claim 1, wherein the system is configured to perform continuous real time monitoring of levels of the at least two different types of cytokines in the fluid.

6.-11. (canceled)

12. The system of claim 1, further configured to provide a fluid flow connection to an intravenous device configured to inject the plurality of nucleic acid-based nanosensors into the fluid.

13. (canceled)

14. The system of claim 1, further comprising:
a plurality of fluid channels configured to flow the fluid comprising the plurality of cytokines, the plurality of nucleic acid-based nanosensors being inside at least one fluid channel of the plurality of fluid channels;
the system being configured to permit photoacoustic measurement of the fluid in the at least one fluid channel.

15.-17. (canceled)

18. A method for multiplexed photoacoustic detection of cytokines, the method comprising:
flowing a fluid comprising a plurality of nucleic acid-based nanosensors each comprising at least two sensor arms, each arm of the at least two sensor arms comprising at least one photoacoustic reporter chromophore and a cytokine receptor, each arm being configured, in the presence of a detected cytokine in a fluid, to induce stacking of the at least one photoacoustic reporter chromophore with at least one photoacoustic reporter chromophore in another one of the at least two sensor arms, thereby increasing a photoacoustic signal emitted by the nucleic acid based nanosensor, and each arm being further configured to induce unstacking of the at least one photoacoustic reporter chromophore when the cytokine is not present, thereby decreasing the photoacoustic signal emitted;
the plurality of nucleic-acid based nanosensors comprising cytokine receptors for at least two different types of cytokines; and
performing a photoacoustic measurement of the fluid to detect the photoacoustic signals emitted by the nucleic acid based nanosensors and to thereby distinguish a differential photoacoustic signal indicating a stacked or unstacked state of the at least one photoacoustic reporter chromophore of the nucleic acid based nanosensors, for each of the at least two different types of cytokines.

19. (canceled)

20. The method of claim 18, comprising detecting two or more of: IFNγ, IL-6, and IL-1.

21. (canceled)

22. The method of claim 18, comprising performing continuous real time monitoring of levels of the at least two different types of cytokines in the fluid.

23. The method of claim 18, further comprising:
injecting the plurality of nucleic acid-based nanosensors into the fluid.

24. The method of claim 23, wherein the plurality of nucleic acid-based nanosensors comprise at least part of an injectable agent, the method further comprising performing spatially resolved imaging of the at least two different types of cytokines in a living subject.

25. The method of claim 18, further comprising:
flowing the fluid comprising a plurality of nucleic-acid based nanosensors through at least one fluid channel; and
performing a photoacoustic measurement of the fluid in the fluid channel to detect the photoacoustic signals emitted by the nucleic acid based nanosensors.

26. A nucleic acid-based nanosensor for photoacoustic detection of cytokines, the nanosensor comprising:
two DNA origami-based sensor arms, each sensor arm comprising at least one photoacoustic reporter chromophore and a cytokine receptor, each sensor arm being configured, in the presence of a detected cytokine in a fluid, to induce stacking of the at least one photoacoustic reporter chromophore with at least one photoacoustic reporter chromophore in another one of the two sensor arms, thereby increasing a photoacoustic signal emitted by the nucleic acid based nanosensor, and each sensor arm being further configured to induce unstacking of the at least one photoacoustic reporter chromophore when the cytokine is not present, thereby decreasing the photoacoustic signal emitted;
the two sensor arms being attached at a central hinge configured to perform the inducing of the stacking in the presence of the detected cytokine, and the inducing of the unstacking when the cytokine is not present, and comprising modification sites upon which the cytokine receptors and the at least one photoacoustic reporter chromophores are installed.

27. The nucleic acid-based nanosensor of claim 26, wherein the nanosensor is configured to induce the stacking of the at least one photoacoustic reporter chromophore through a two-step binding process comprising a first binding of a first cytokine receptor in a first sensor arm with the detected cytokine, and a second binding of a second cytokine receptor in a second sensor arm with the complex of the first cytokine receptor and the detected cytokine.

28. The nucleic acid-based nanosensor of claim 26, wherein the nanosensor is configured to detect one or more of: IFNγ, IL-6, and IL-1.

29. The nucleic acid-based nanosensor of claim 28, comprising at least one of the following pairs of cytokine receptor pairs, one cytokine receptor of each pair being conjugated on a first arm of the at least two sensor arms, and another cytokine receptor of each pair being conjugated on a second arm of the at least two sensor arms, the cytokine receptor pairs comprising: IFNγR1 and IFNγR2; IL-6R and gp130; and IL-1R1 and IL-1RAcP.

30. The nucleic acid-based nanosensor of claim 26, comprising protective shielding from biological conditions.

31. The nucleic acid-based nanosensor of claim 26, further comprising a single stranded DNA tether between the two arms of the tweezer structure.

32. The nucleic acid-based nanosensor of claim 26, wherein the at least one photoacoustic reporter chromophore comprises at least one of an indocyanine green analog, a phthalocyanine chromophore, and a BODIPY analog.

33. The nucleic acid-based nanosensor of claim 32, wherein the at least one photoacoustic reporter chromophore comprises an indocyanine green analog, and further comprising a molecular scaffold.

34. The nucleic acid-based nanosensor of claim 26, comprising at least part of an injectable agent configured to permit spatially resolved imaging of at least one cytokine in a living subject.

35. A device for multiplexed photoacoustic detection of cytokines, the device comprising:
a plurality of nucleic acid-based nanosensors each comprising at least two sensor arms, each arm of the at least two sensor arms comprising at least one photoacoustic reporter chromophore and a cytokine receptor, each arm being configured, in the presence of a detected cytokine in a fluid, to induce stacking of the at least one photoacoustic reporter chromophore with at least one photoacoustic reporter chromophore in another one of the at least two sensor arms, thereby increasing a photoacoustic signal emitted by the nucleic acid based nanosensor, and each arm being further configured to induce unstacking of the at least one photoacoustic reporter chromophore when the cytokine is not present, thereby decreasing the photoacoustic signal emitted;
the plurality of nucleic-acid based nanosensors comprising cytokine receptors for at least two different types of cytokines;
the device being configured to permit detection of the photoacoustic signals emitted by the nucleic acid based nanosensors by a photoacoustic measurement system and to thereby permit distinguishing of a differential photoacoustic signal indicating a stacked or unstacked state of the at least one photoacoustic reporter chromophore of the nucleic acid based nanosensors, for each of the at least two different types of cytokines.

36. The device of claim 35, further comprising:
a plurality of fluid channels configured to flow the fluid comprising the plurality of cytokines, the plurality of nucleic acid-based nanosensors being inside at least one fluid channel of the plurality of fluid channels;
the system being configured to permit photoacoustic measurement of the fluid in the at least one fluid channel.

37.-39. (canceled)
